SEQUENCE LISTING (10 sequences)

SEQ ID NO: 1 - DNA, 1080 bp, Homo sapiens
  source 1..1080: mol_type = genomic DNA
atggcgggacacctggcttcggatttcgccttctcgccccctccaggtggtggaggtgatgggccaggggggccggagccgggctgggttgatcctcggacctggctaagcttccaaggccctcctggagggccaggaatcgggccgggggttgggccaggctctgaggtgtgggggattcccccatgccccccgccgtatgagttctgtggggggatggcgtactgtgggccccaggttggagtggggctagtgccccaaggcggcttggagacctctcagcctgagggtgaagcaggagtcggggtggagagcaactccgatggggcctccccggagccctgcaccgtcacccctggtgccgtgaagctggagaaggagaagctggagcaaaacccggaggagtcccaggacatcaaagctctgcagaaagaactcgagcaatttgccaagctcctgaagcagaagaggatcaccctgggatatacacaggccgatgtggggctcaccctgggggttctatttgggaaggtattcagccaaacgaccatctgccgctttgaggctctgcagcttagcttcaagaacatgtgtaagctgcggcccttgctgcagaagtgggtggaggaagctgacaacaatgaaaatcttcaggagatatgcaaagcagaaaccctcgtgcaggcccgaaagagaaagcgaaccagtatcgagaaccgagtgagaggcaacctggagaatttgttcctgcagtgcccgaaacccacactgcagcagatcagccacatcgcccagcagcttgggctcgagaaggatgtggtccgagtgtggttctgtaaccggcgccagaagggcaagcgatcaagcagcgactatgcacaacgagaggattttgaggctgctgggtctcctttctcagggggaccagtgtcctttcctctggccccagggccccattttggtaccccaggctatgggagccctcacttcactgcactgtactcctcggtccctttccctgagggggaagcctttccccctgtctctgtcaccactctgggctctcccatgcattcaaac

SEQ ID NO: 2 - DNA, 1410 bp, Homo sapiens
  source 1..1410: mol_type = genomic DNA
atggctgtcagcgacgcgctgctcccatctttctccacgttcgcgtctggcccggcgggaagggagaagacactgcgtcaagcaggtgccccgaataaccgctggcgggaggagctctcccacatgaagcgacttcccccagtgcttcccggccgcccctatgacctggcggcggcgaccgtggccacagacctggagagcggcggagccggtgcggcttgcggcggtagcaacctggcgcccctacctcggagagagaccgaggagttcaacgatctcctggacctggactttattctctccaattcgctgacccatcctccggagtcagtggccgccaccgtgtcctcgtcagcgtcagcctcctcttcgtcgtcgccgtcgagcagcggccctgccagcgcgccctccacctgcagcttcacctatccgatccgggccgggaacgacccgggcgtggcgccgggcggcacgggcggaggcctcctctatggcagggagtccgctccccctccgacggctcccttcaacctggcggacatcaacgacgtgagcccctcgggcggcttcgtggccgagctcctgcggccagaattggacccggtgtacattccgccgcagcagccgcagccgccaggtggcgggctgatgggcaagttcgtgctgaaggcgtcgctgagcgcccctggcagcgagtacggcagcccgtcggtcatcagcgtcagcaaaggcagccctgacggcagccacccggtggtggtggcgccctacaacggcgggccgccgcgcacgtgccccaagatcaagcaggaggcggtctcttcgtgcacccacttgggcgctggaccccctctcagcaatggccaccggccggctgcacacgacttccccctggggcggcagctccccagcaggactaccccgaccctgggtcttgaggaagtgctgagcagcagggactgtcaccctgccctgccgcttcctcccggcttccatccccacccggggcccaattacccatccttcctgcccgatcagatgcagccgcaagtcccgccgctccattaccaagagctcatgccacccggttcctgcatgccagaggagcccaagccaaagaggggaagacgatcgtggccccggaaaaggaccgccacccacacttgtgattacgcgggctgcggcaaaacctacacaaagagttcccatctcaaggcacacctgcgaacccacacaggtgagaaaccttaccactgtgactgggacggctgtggatggaaattcgcccgctcagatgaactgaccaggcactaccgtaaacacacggggcaccgcccgttccagtgccaaaaatgcgaccgagcattttccaggtcggaccacctcgccttacacatgaagaggcatttt

SEQ ID NO: 3 - DNA, 954 bp, Homo sapiens
  source 1..954: mol_type = genomic DNA
atgtacaacatgatggagacggagctgaagccgccgggcccgcagcaaacttcggggggcggcggcggcaactccaccgcggcggcggccggcggcaaccagaaaaacagcccggaccgcgtcaagcggcccatgaatgccttcatggtgtggtcccgcgggcagcggcgcaagatggcccaggagaaccccaagatgcacaactcggagatcagcaagcgcctgggcgccgagtggaaacttttgtcggagacggagaagcggccgttcatcgacgaggctaagcggctgcgagcgctgcacatgaaggagcacccggattataaataccggccccggcggaaaaccaagacgctcatgaagaaggataagtacacgctgcccggcgggctgctggcccccggcggcaatagcatggcgagcggggtcggggtgggcgccggcctgggcgcgggcgtgaaccagcgcatggacagttacgcgcacatgaacggctggagcaacggcagctacagcatgatgcaggaccagctgggctacccgcagcacccgggcctcaatgcgcacggcgcagcgcagatgcagcccatgcaccgctacgacgtgagcgccctgcagtacaactccatgaccagctcgcagacctacatgaacggctcgcccacctacagcatgtcctactcgcagcagggcacccctggcatggctcttggctccatgggttcggtggtcaagtccgaggccagctccagcccccctgtggttacctcttcctcccactccagggcgccctgccaggccggggacctccgggacatgatcagcatgtatctccccggcgccgaggtgccggaacccgccgcccccagcagacttcacatgtcccagcactaccagagcggcccggtgcccggcacggccattaacggcacactgcccctctcacacatgtga

SEQ ID NO: 4 - protein, 360 aa, Homo sapiens
MAGHLASDFAFSPPPGGGGDGPGGPEPGWVDPRTWLSFQGPPGGPGIGPGVGPGSEVWGIPPCPPPYEFCGGMAYCGPQVGVGLVPQGGLETSQPEGEAGVGVESNSDGASPEPCTVTPGAVKLEKEKLEQNPEESQDIKALQKELEQFAKLLKQKRITLGYTQADVGLTLGVLFGKVFSQTTICRFEALQLSFKNMCKLRPLLQKWVEEADNNENLQEICKAETLVQARKRKRTSIENRVRGNLENLFLQCPKPTLQQISHIAQQLGLEKDVVRVWFCNRRQKGKRSSSDYAQREDFEAAGSPFSGGPVSFPLAPGPHFGTPGYGSPHFTALYSSVPFPEGEAFPPVSVTTLGSPMHSN

SEQ ID NO: 5 - protein, 470 aa, Homo sapiens
MAVSDALLPSFSTFASGPAGREKTLRQAGAPNNRWREELSHMKRLPPVLPGRPYDLAAATVATDLESGGAGAACGGSNLAPLPRRETEEFNDLLDLDFILSNSLTHPPESVAATVSSSASASSSSSPSSSGPASAPSTCSFTYPIRAGNDPGVAPGGTGGGLLYGRESAPPPTAPFNLADINDVSPSGGFVAELLRPELDPVYIPPQQPQPPGGGLMGKFVLKASLSAPGSEYGSPSVISVSKGSPDGSHPVVVAPYNGGPPRTCPKIKQEAVSSCTHLGAGPPLSNGHRPAAHDFPLGRQLPSRTTPTLGLEEVLSSRDCHPALPLPPGFHPHPGPNYPSFLPDQMQPQVPPLHYQELMPPGSCMPEEPKPKRGRRSWPRKRTATHTCDYAGCGKTYTKSSHLKAHLRTHTGEKPYHCDWDGCGWKFARSDELTRHYRKHTGHRPFQCQKCDRAFSRSDHLALHMKRHF

SEQ ID NO: 6 - protein, 317 aa, Homo sapiens
MYNMMETELKPPGPQQTSGGGGGNSTAAAAGGNQKNSPDRVKRPMNAFMVWSRGQRRKMAQENPKMHNSEISKRLGAEWKLLSETEKRPFIDEAKRLRALHMKEHPDYKYRPRRKTKTLMKKDKYTLPGGLLAPGGNSMASGVGVGAGLGAGVNQRMDSYAHMNGWSNGSYSMMQDQLGYPQHPGLNAHGAAQMQPMHRYDVSALQYNSMTSSQTYMNGSPTYSMSYSQQGTPGMALGSMGSVVKSEASSSPPVVTSSSHSRAPCQAGDLRDMISMYLPGAEVPEPAAPSRLHMSQHYQSGPVPGTAINGTLPLSHM

SEQ ID NO: 7 - DNA, 747 bp, synthetic construct
  source 1..747: mol_type = other DNA
  misc_feature 1..747: note = Tet-On 3G
atgtctagactggacaagagcaaagtcataaactctgctctggaattactcaatggagtcggtatcgaaggcctgacgacaaggaaactcgctcaaaagctgggagttgagcagcctaccctgtactggcacgtgaagaacaagcgggccctgctcgatgccctgccaatcgagatgctggacaggcatcatacccactcctgccccctggaaggcgagtcatggcaagactttctgcggaacaacgccaagtcataccgctgtgctctcctctcacatcgcgacggggctaaagtgcatctcggcacccgcccaacagagaaacagtacgaaaccctggaaaatcagctcgcgttcctgtgtcagcaaggcttctccctggagaacgcactgtacgctctgtccgccgtgggccactttacactgggctgcgtattggaggaacaggagcatcaagtagcaaaagaggaaagagagacacctaccaccgattctatgcccccacttctgaaacaagcaattgagctgttcgaccggcagggagccgaacctgccttccttttcggcctggaactaatcatatgtggcctggagaaacagctaaagtgcgaaagcggcgggccgaccgacgcccttgacgattttgacttagacatgctcccagccgatgcccttgacgactttgaccttgatatgctgcctgctgacgctcttgacgattttgaccttgacatgctccccgggtaa

SEQ ID NO: 8 - protein, 248 aa, synthetic construct
  REGION 1..248: note = Tet-On 3G
MSRLDKSKVINSALELLNGVGIEGLTTRKLAQKLGVEQPTLYWHVKNKRALLDALPIEMLDRHHTHSCPLEGESWQDFLRNNAKSYRCALLSHRDGAKVHLGTRPTEKQYETLENQLAFLCQQGFSLENALYALSAVGHFTLGCVLEEQEHQVAKEERETPTTDSMPPLLKQAIELFDRQGAEPAFLFGLELIICGLEKQLKCESGGPTDALDDFDLDMLPADALDDFDLDMLPADALDDFDLDMLPG

SEQ ID NO: 9 - DNA, 379 bp, synthetic construct
  source 1..379: mol_type = other DNA
  misc_feature 1..379: note = TRE3G Promoter
gagtttactccctatcagtgatagagaacgtatgaagagtttactccctatcagtgatagagaacgtatgcagactttactccctatcagtgatagagaacgtataaggagtttactccctatcagtgatagagaacgtatgaccagtttactccctatcagtgatagagaacgtatctacagtttactccctatcagtgatagagaacgtatatccagtttactccctatcagtgatagagaacgtataagctttaggcgtgtacggtgggcgcctataaaagcagagctcgtttagtgaaccgtcagatcgcctggagcaattccacaacacttttgtcttataccaactttccgtaccacttcctaccctcgtaaa

SEQ ID NO: 10 - DNA, 8342 bp, synthetic construct
  source 1..8342: mol_type = other DNA
  misc_feature 1..8342: note = hOKS Vector
ctttcctgcgttatcccctgattctgtggataaccgtattaccgctaggcgatcgcttaattaactagcatggatctcggggacgtctaactactaagcgagagtagggaactgccaggcatcaaataaaacgaaaggctcagtcggaagactgggcctttcgttttatctgttgtttgtcggtgaacgctctcctgagtaggacaaatccgccgggagcggatttgaacgttgtgaagcaacggcccggagggtggcgggcaggacgcccgccataaactgccaggcatcaaactaagcagaaggccatcctgacggatggcctttttgcgtttctacaaactcttcctgttagttagttacttaagctcgggccccaaataatgattttattttgactgatagtgacctgttcgttgcaacaaattgataagcaatgcttttttataatgccaactttgtacaaaaaagcaggctttaaaggaaccaattcagtcgataactataacggtcctaaggtagcgatttaaatacgcgctctcttaaggtagccccgggacgcgggtagcgaaagctcagatccgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagtcgaataatcaaacagaccagattgtctgtttgtttcgacaagctctagcctcgaagatctcgagccaccatgtctagactggacaagagcaaagtcataaactctgctctggaattactcaatggagtcggtatcgaaggcctgacgacaaggaaactcgctcaaaagctgggagttgagcagcctaccctgtactggcacgtgaagaacaagcgggccctgctcgatgccctgccaatcgagatgctggacaggcatcatacccactcctgccccctggaaggcgagtcatggcaagactttctgcggaacaacgccaagtcataccgctgtgctctcctctcacatcgcgacggggctaaagtgcatctcggcacccgcccaacagagaaacagtacgaaaccctggaaaatcagctcgcgttcctgtgtcagcaaggcttctccctggagaacgcactgtacgctctgtccgccgtgggccactttacactgggctgcgtattggaggaacaggagcatcaagtagcaaaagaggaaagagagacacctaccaccgattctatgcccccacttctgaaacaagcaattgagctgttcgaccggcagggagccgaacctgccttccttttcggcctggaactaatcatatgtggcctggagaaacagctaaagtgcgaaagcggcgggccgaccgacgcccttgacgattttgacttagacatgctcccagccgatgcccttgacgactttgaccttgatatgctgcctgctgacgctcttgacgattttgaccttgacatgctccccgggtaataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatccggctgcctcgcgcctcgagtttactccctatcagtgatagagaacgtatgaagagtttactccctatcagtgatagagaacgtatgcagactttactccctatcagtgatagagaacgtataaggagtttactccctatcagtgatagagaacgtatgaccagtttactccctatcagtgatagagaacgtatctacagtttactccctatcagtgatagagaacgtatatccagtttactccctatcagtgatagagaacgtataagctttaggcgtgtacggtgggcgcctataaaagcagagctcgtttagtgaaccgtcagatcgcctggagcaattccacaacacttttgtcttataccaactttccgtaccacttcctaccctcgtaaagtcgacgctagcggatccagaattccaccatggcgggacacctggcttcggatttcgccttctcgccccctccaggtggtggaggtgatgggccaggggggccggagccgggctgggttgatcctcggacctggctaagcttccaaggccctcctggagggccaggaatcgggccgggggttgggccaggctctgaggtgtgggggattcccccatgccccccgccgtatgagttctgtggggggatggcgtactgtgggccccaggttggagtggggctagtgccccaaggcggcttggagacctctcagcctgagggtgaagcaggagtcggggtggagagcaactccgatggggcctccccggagccctgcaccgtcacccctggtgccgtgaagctggagaaggagaagctggagcaaaacccggaggagtcccaggacatcaaagctctgcagaaagaactcgagcaatttgccaagctcctgaagcagaagaggatcaccctgggatatacacaggccgatgtggggctcaccctgggggttctatttgggaaggtattcagccaaacgaccatctgccgctttgaggctctgcagcttagcttcaagaacatgtgtaagctgcggcccttgctgcagaagtgggtggaggaagctgacaacaatgaaaatcttcaggagatatgcaaagcagaaaccctcgtgcaggcccgaaagagaaagcgaaccagtatcgagaaccgagtgagaggcaacctggagaatttgttcctgcagtgcccgaaacccacactgcagcagatcagccacatcgcccagcagcttgggctcgagaaggatgtggtccgagtgtggttctgtaaccggcgccagaagggcaagcgatcaagcagcgactatgcacaacgagaggattttgaggctgctgggtctcctttctcagggggaccagtgtcctttcctctggccccagggccccattttggtaccccaggctatgggagccctcacttcactgcactgtactcctcggtccctttccctgagggggaagcctttccccctgtctctgtcaccactctgggctctcccatgcattcaaacggggcgcgtaaaattgtcgctcctgtcaaacaaactcttaactttgatttactcaaactggctggggatgtagaaagcaatccaggtccaggggcgcgtggtatggctgtcagcgacgcgctgctcccatctttctccacgttcgcgtctggcccggcgggaagggagaagacactgcgtcaagcaggtgccccgaataaccgctggcgggaggagctctcccacatgaagcgacttcccccagtgcttcccggccgcccctatgacctggcggcggcgaccgtggccacagacctggagagcggcggagccggtgcggcttgcggcggtagcaacctggcgcccctacctcggagagagaccgaggagttcaacgatctcctggacctggactttattctctccaattcgctgacccatcctccggagtcagtggccgccaccgtgtcctcgtcagcgtcagcctcctcttcgtcgtcgccgtcgagcagcggccctgccagcgcgccctccacctgcagcttcacctatccgatccgggccgggaacgacccgggcgtggcgccgggcggcacgggcggaggcctcctctatggcagggagtccgctccccctccgacggctcccttcaacctggcggacatcaacgacgtgagcccctcgggcggcttcgtggccgagctcctgcggccagaattggacccggtgtacattccgccgcagcagccgcagccgccaggtggcgggctgatgggcaagttcgtgctgaaggcgtcgctgagcgcccctggcagcgagtacggcagcccgtcggtcatcagcgtcagcaaaggcagccctgacggcagccacccggtggtggtggcgccctacaacggcgggccgccgcgcacgtgccccaagatcaagcaggaggcggtctcttcgtgcacccacttgggcgctggaccccctctcagcaatggccaccggccggctgcacacgacttccccctggggcggcagctccccagcaggactaccccgaccctgggtcttgaggaagtgctgagcagcagggactgtcaccctgccctgccgcttcctcccggcttccatccccacccggggcccaattacccatccttcctgcccgatcagatgcagccgcaagtcccgccgctccattaccaagagctcatgccacccggttcctgcatgccagaggagcccaagccaaagaggggaagacgatcgtggccccggaaaaggaccgccacccacacttgtgattacgcgggctgcggcaaaacctacacaaagagttcccatctcaaggcacacctgcgaacccacacaggtgagaaaccttaccactgtgactgggacggctgtggatggaaattcgcccgctcagatgaactgaccaggcactaccgtaaacacacggggcaccgcccgttccagtgccaaaaatgcgaccgagcattttccaggtcggaccacctcgccttacacatgaagaggcattttggggcgcgtaaaattgtcgctcctgtcaaacaaactcttaactttgatttactcaaactggctggggatgtagaaagcaatccaggtccaggggcgcgtggtatgtacaacatgatggagacggagctgaagccgccgggcccgcagcaaacttcggggggcggcggcggcaactccaccgcggcggcggccggcggcaaccagaaaaacagcccggaccgcgtcaagcggcccatgaatgccttcatggtgtggtcccgcgggcagcggcgcaagatggcccaggagaaccccaagatgcacaactcggagatcagcaagcgcctgggcgccgagtggaaacttttgtcggagacggagaagcggccgttcatcgacgaggctaagcggctgcgagcgctgcacatgaaggagcacccggattataaataccggccccggcggaaaaccaagacgctcatgaagaaggataagtacacgctgcccggcgggctgctggcccccggcggcaatagcatggcgagcggggtcggggtgggcgccggcctgggcgcgggcgtgaaccagcgcatggacagttacgcgcacatgaacggctggagcaacggcagctacagcatgatgcaggaccagctgggctacccgcagcacccgggcctcaatgcgcacggcgcagcgcagatgcagcccatgcaccgctacgacgtgagcgccctgcagtacaactccatgaccagctcgcagacctacatgaacggctcgcccacctacagcatgtcctactcgcagcagggcacccctggcatggctcttggctccatgggttcggtggtcaagtccgaggccagctccagcccccctgtggttacctcttcctcccactccagggcgccctgccaggccggggacctccgggacatgatcagcatgtatctccccggcgccgaggtgccggaacccgccgcccccagcagacttcacatgtcccagcactaccagagcggcccggtgcccggcacggccattaacggcacactgcccctctcacacatgtgagtttaaatctaggaattcgtttaaactctagacgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggccgcggctcgagggcgcgccctagacccagctttcttgtacaaagttggcattataagaaagcattgcttatcaatttgttgcaacgaacaggtcactatcagtcaaaataaaatcattatttgccatccagctgcagctctggcccgtgtctcaaaatctctgatgttacattgcacaagataaaaatatatcatcatgaacaataaaactgtctgcttacataaacagtaatacaaggggtgttatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgtccctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacattattcagattgggccccgttccactgagcgtcagacccggtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgtt

(19) United States
(12) Patent Application Publication
(10) Pub. No.: US 2023/0302044 A1
(43) Pub. Date: Sep. 28, 2023

(54) COMPOSITION TO INCREASE CELLULARLONGEVITY

(71) Applicant: Steve Reilly, Harrison, NY (US)

(72) Inventor: Steve REILLY, Harrison, NY (US)

(21) Appl. No.: 18/191,718

(22) Filed: Mar. 28, 2023

Related U.S. Application Data
(60) Provisional application No. 63/324,480, filed on Mar. 28, 2022.

(51) Int. Cl. A61K 31/785; A61K 31/7088; A61P 43/00

(52) U.S. Cl. CPC A61K 31/785; A61K 31/7088; A61P 43/00

(57) ABSTRACT

The present disclosure provides compositions and methods to increase cellular longevity. In some embodiments, the compositions comprise administering a polynucleotide that encodes transcription factors to a subject in need thereof. In some embodiments, the composition increases telomere length compared to baseline. In some embodiments, the polynucleotide increases anagen hair follicles compared to baseline.

### CROSS-REFERENCE TO RELATED APPLICATIONS

This application claims benefit of priority to U.S. Provisional Patent Application No. 63/324,480, filed Mar. 28, 2022, the disclosure of which is incorporated herein by reference in its entirety for all purposes.

### STATEMENT REGARDING THE SEQUENCE LISTING

The Sequence Listing XML associated with this application is provided in XML file format and is hereby incorporated by reference into the specification. The name of the XML file containing the Sequence Listing XML is SRLY_001_01US_ST26.xml. The XML file is 25,430 bytes, and created on Mar. 28, 2023, and is being submitted electronically via USPTO Patent Center.

### FIELD

The present disclosure relates to methods of reprogramming a cell in vivo or in vitro through PEI-mediated delivery of a polynucleotide that encodes one or more transcription factors, such as Yamanaka factors (Oct3/4, Sox2, Kl4, and c-Myc). The disclosure further relates to a reprogramming a somatic cell according to the methods as defined herein. Also provided are compositions for use in treatment or rejuvenation, as well as methods for screening age modulating agents, factors and/or cellular processes, comprising the methods and a reprogrammed somatic cell as defined herein.

### BACKGROUND

Ageing is characterized by a gradual loss of function occurring at the molecular, cellular, tissue and organismal levels. As organisms age, the pattern of DNA methylation at the chromatin level changes with some sites gaining and some sites losing this mark. DNA methylation is an epigenetic modification that plays many roles in mammalian cells ranging from transposable element silencing to X chromosome inactivation and, as such, changes and progressive accumulation of epigenetic marks are associated with aberrant gene expression and regulation, stem cell exhaustion, senescence and dysregulated tissue homeostasis. In addition, telomeres begin to shorten with age. Telomere length serves as a useful indicator in the study of chromosomal stability, proliferative capacity and aging process of the cells.

These changes are relatively consistent between individuals and can be used to predict age. Such predictors (e.g., the Horvath epigenetic clock) produce a value called DNA methylation age (also known as epigenetic age), which is thought to represent the biological age of an individual or a tissue. Lifestyle factors that affect the ageing process (e.g., diet) can also affect DNA methylation age and telomere length. Nevertheless, the biology underlying the epigenetic clock, DNA methylation age, and telomere length remains unclear.

During the process of induced pluripotent stem (iPS) cell reprogramming, somatic cells are converted or de-differentiated into pluripotent stem cells. Gene expression profiling has revealed 3 phases of reprograming: initiation, maturation, and stabilization. While the initiation phase is characterized by an immediate mesenchymal-to-epithelial transition, the expression of a subset of pluripotency-associated genes is detected in the maturation phase. The resulting iPS cells are similar to natural pluripotent stem cells (e.g., embryonic stem (ES) cells) in many aspects, including in their ability to differentiate into multiple cell types. However, during iPS cell reprogramming, DNA methylation age is reset to zero years old regardless of the age of the donor tissue from which the somatic cell was obtained. As such, the process of iPS cell reprogramming resets the epigenetic signature of the somatic cell to an embryonic-like state and causes loss of somatic cell lineage identity. While in vitro iPS cell reprogramming has made great strides, similar advancements in in vivo cell reprogramming are lacking.

There is, therefore, a need to produce reprogrammed cells which have reduced DNA methylation age and increased telomere length but retain lineage identity. Such a method to produce reprogrammed cells will find use in numerous therapeutic applications as well as in the treatment and/or amelioration of age-related or degenerative diseases and disorders.

### SUMMARY

The present disclosure solves the aforementioned problems by providing a composition to increase cellular longevity comprising a polyethylenimine (PEI) formulation and a polynucleotide. In some cases, the polynucleotide encodes one or more heterologous polynucleotide(s) encoding one or more Yamanaka Factors, wherein the one or more polynucleotide(s) encoding the one or more Yamanaka Factors is operably linked to a promoter that is not in nature associated with the one or more polynucleotide(s) encoding the one or more Yamanaka Factors, said promoter directly or indirectly induced. In some cases, the polynucleotide expresses the polynucleotide(s) encoding one or more Yamanaka Factors under conditions that induce the promoter operably linked to the polynucleotide(s) encoding the one or more Yamanaka Factors. In some cases, the polynucleotide is bound to the cationic monomers and is substantially located within the core of the PEI formulation. In some cases, the one or more heterologous polynucleotide(s) encoding one or more Yamanaka Factors. In some cases, the one or more heterologous polynucleotide(s) encodes an octamer-binding transcription factor 4 (OCT4). In some cases, the one or more polynucleotide(s) encoding the OCT4 is operably linked to an inducible promoter that is not in nature associated with the one or more polynucleotide(s) encoding the OCT4, said promoter directly or indirectly induced. In some cases, one or more heterologous polynucleotide(s) encodes a Kruppel Like Factor 4 (KLF4). In some cases, the one or more polynucleotide(s) encoding the KLF4 is operably linked to a promoter that is not in nature associated with the one or more polynucleotide(s) encoding the KLF4, said promoter directly or indirectly induced. In some cases, one or more heterologous polynucleotide(s) encodes an SRY-box 2 (SOX2). In some cases, the one or more polynucleotide(s) encoding the SOX2 is operably linked to an inducible promoter that is not in nature associated with the one or more polynucleotide(s) encoding the SOX2, said promoter directly or indirectly induced. In some cases, the polynucleotide expresses the polynucleotide(s) encoding OCT4 under conditions that induce the promoter operably linked to the polynucleotide(s) encoding OCT4; the polynucleotide(s) encoding KLF4 under conditions that induce the promoter operably linked to the polynucleotide(s) encoding KLF4; the polynucleotide(s) encoding SOX2 under conditions that include the promoter operably linked to the polynucleotide(s) encoding SOX2. In some cases, the polynucleotide is bound to the cationic monomers and is substantially located within the core of the PEI formulation. In some cases, the promoter operably linked to the polynucleotide(s) encoding OCT4, the promoter operably linked to KLF4, and the promoter operably linked to the polynucleotide(s) encoding SOX2 are the same promoter. In some cases, at least one polynucleotide is operably linked to a directly or indirectly inducible promoter that is induced by a chemical and/or nutritional inducer. In some cases, the chemical and/or nutritional inducer is selected from arabinose, IPTG, tetracycline, and rhamnose. In some cases, at least one polynucleotide is under the control of a Tet promoter sequence and a Tet repressor polynucleotide. In some cases, the OCT4 polynucleotide shares at least 80%, at least 85%, at least 90%, at least 95%, at least 98%, at least 99%, or 100% identity to SEQ ID NO: 1. In some cases, the polynucleotide encodes a OCT4 polypeptide shares at least 80%, at least 85%, at least 90%, at least 95%, at least 98%, at least 99%, or 100% identity to SEQ ID NO: 4. In some cases, the SOX2 polynucleotide shares at least 80%, at least 85%, at least 90%, at least 95%, at least 98%, at least 99%, or 100% identity to SEQ ID NO: 3. In some cases, the polynucleotide encodes a SOX2 polypeptide shares at least 80%, at least 85%, at least 90%, at least 95%, at least 98%, at least 99%, or 100% identity to SEQ ID NO: 6. In some cases, the KLF4 polynucleotide shares at least 80%, at least 85%, at least 90%, at least 95%, at least 98%, at least 99%, or 100% identity to SEQ ID NO: 2. In some cases, the polynucleotide encodes a KLF4 polypeptide shares at least 80%, at least 85%, at least 90%, at least 95%, at least 98%, at least 99%, or 100% identity to SEQ ID NO: 5. In some cases, the composition is administrated to a subject in need thereof. In some cases, the cell is a non-human primate cell. In some cases, the cell is a human cell. In some cases, the PEI formulation and polynucleotide are administered systemically. In some cases, the PEI formulation and polynucleotide are administered intravenously. In some cases, the PEI formulation and polynucleotide are administered in a single dose. In some cases, the PEI formulation and polynucleotide are administered in multiple doses. In some cases, the subject is administered a dosage of at least about 0.001 mg/kg to about 1000 mg/kg of doxycycline for at least 1 day per week and water only for the remaining days for at least 2 weeks. In some cases, the subject is administered a dosage of at least about 0.001 mg/kg to about 1000 mg/kg of doxycycline for at least 1 day per week and water only for the remaining days for at least 8 weeks. In some cases, the subject is administered a dosage of at least about 0.001 mg/kg to about 1000 mg/kg of doxycycline for at least 1 day per week and water only for the remaining days for at least 12 weeks. In some cases, the subject is administered a dosage of at least about 0.001 mg/kg to about 1000 mg/kg of doxycycline for at least 1 day per week and water only for the remaining days for at least 26 weeks. In some cases, the subject is administered a dosage of at least about 0.001 mg/kg to about 1000 mg/kg of doxycycline for at least 1 day per week and water only for the remaining days for at least 52 weeks. In some cases, the cell has increased CpG promoter hypermethylation compared to baseline. In some cases, the cell has increased telomere length compared to baseline. In some cases, the cell has from about 10% to about 50% increased median telomere length compared to baseline. In some cases, the cell has about 30% increased median telomere length compared to baseline. In some cases, the cell has from about 10% to about 50% increased 20^{th} percentile telomere length compared to baseline. In some cases, the cell has about 30% increased 20^{th} percentile telomere length compared to baseline. In some cases, the cell has about 30% increased 20^{th} percentile telomere length compared to baseline. In some cases, the cell has about 10% decreased telomeres 3 Kilo base pairs (Kbp) in length compared to baseline. In some cases, the composition increases the density of anagen follicles in a subject. In some cases, the composition increases the density of catagen follicles in a subject. In some cases, the composition decreases adnexal atrophy in a subject. In some cases, the composition decreases mononuclear perivascular inflammatory infiltrate in the dermis in a subject. In some cases, the polynucleotide and PEI comprise a N:P ratio. In some cases, the N:P ratio is from about 1:1 to about 10:1. In some cases, the N:P ratio is about 1:1 to about 1:10. In some cases, the N:P ratio is about 5:1. In some cases, the composition increases fertility in a subject. In some cases, the composition increases lifespan, decreases hair loss in a subject by about 10%, and/or increases skin elasticity by about 10% in a subject compared to a subject that was not administered the composition.

In another aspect, the disclosure provides for a method to increase cellular longevity comprising administering a polyethylenimine (PEI) formulation and a polynucleotide to a subject in need thereof. In some cases, the polynucleotide encodes one or more heterologous polynucleotide(s) encoding one or more Yamanaka Factors, wherein the one or more polynucleotide(s) encoding the one or more Yamanaka Factors is operably linked to a promoter that is not in nature associated with the one or more polynucleotide(s) encoding the one or more Yamanaka Factors, said promoter directly or indirectly induced. In some cases, the polynucleotide expresses the polynucleotide(s) encoding one or more Yamanaka Factors under conditions that induce the promoter operably linked to the polynucleotide(s) encoding the one or more Yamanaka Factors. In some cases, the polynucleotide is bound to the cationic monomers and is substantially located within the core of the PEI formulation. In some cases, the one or more heterologous polynucleotide(s) encoding one or more Yamanaka Factors. In some cases, the one or more heterologous polynucleotide(s) encoding an octamer-binding transcription factor 4 (OCT4), wherein the one or more polynucleotide(s) encoding the OCT4 is operably linked to an inducible promoter that is not in nature associated with the one or more polynucleotide(s) encoding the OCT4, said promoter directly or indirectly induced. In some cases, the one or more heterologous polynucleotide(s) encoding a Kruppel Like Factor 4 (KLF4), wherein the one or more polynucleotide(s) encoding the KLF4 is operably linked to a promoter that is not in nature associated with the one or more polynucleotide(s) encoding the KLF4, said promoter directly or indirectly induced. In some cases, the one or more heterologous polynucleotide(s) encoding an SRY-box 2 (SOX2), wherein the one or more polynucleotide(s) encoding the SOX2 is operably linked to an inducible promoter that is not in nature associated with the one or more polynucleotide(s) encoding the SOX2, said promoter directly or indirectly induced. In some cases, the polynucleotide expresses the polynucleotide(s) encoding OCT4 under conditions that induce the promoter operably linked to the polynucleotide(s) encoding OCT4; the polynucleotide(s) encoding KLF4 under conditions that induce the promoter operably linked to the polynucleotide(s) encoding KLF4; the polynucleotide(s) encoding SOX2 under conditions that include the promoter operably linked to the polynucleotide(s) encoding SOX2. In some cases, the polynucleotide is bound to the cationic monomers and is substantially located within the core of the PEI formulation. In some cases, the promoter operably linked to the polynucleotide(s) encoding OCT4, the promoter operably linked to KLF4, and the promoter operably linked to the polynucleotide(s) encoding SOX2 are the same promoter. In some cases, at least one polynucleotide is operably linked to a directly or indirectly inducible promoter that is induced by a chemical and/or nutritional inducer. In some cases, the chemical and/or nutritional inducer is selected from arabinose, IPTG, tetracycline, and rhamnose. In some cases, at least one polynucleotide is under the control of a Tet promoter sequence and a Tet repressor polynucleotide. In some cases, the OCT4 polynucleotide shares at least 80%, at least 85%, at least 90%, at least 95%, at least 98%, at least 99%, or 100% identity to SEQ ID NO: 1. In some cases, the polynucleotide encodes a OCT4 polypeptide shares at least 80%, at least 85%, at least 90%, at least 95%, at least 98%, at least 99%, or 100% identity to SEQ ID NO: 4. In some cases, the SOX2 polynucleotide shares at least 80%, at least 85%, at least 90%, at least 95%, at least 98%, at least 99%, or 100% identity to SEQ ID NO: 3. In some cases, the polynucleotide encodes a SOX2 polypeptide shares at least 80%, at least 85%, at least 90%, at least 95%, at least 98%, at least 99%, or 100% identity to SEQ ID NO: 6. In some cases, the KLF4 polynucleotide shares at least 80%, at least 85%, at least 90%, at least 95%, at least 98%, at least 99%, or 100% identity to SEQ ID NO: 2. In some cases, the polynucleotide encodes a KLF4 polypeptide shares at least 80%, at least 85%, at least 90%, at least 95%, at least 98%, at least 99%, or 100% identity to SEQ ID NO: 5. In some cases, the PEI formulation and the polynucleotide contacts a cell of the subject. In some cases, the subject is a human. In some cases, the cell is a non-human primate cell. In some cases, the cell is a human cell. In some cases, the PEI formulation and polynucleotide are administered systemically. In some cases, the PEI formulation and polynucleotide are administered intravenously. In some cases, the PEI formulation and polynucleotide are administered in a single dose. In some cases, the PEI formulation and polynucleotide are administered in multiple doses. In some cases, the subject is administered a dosage of at least about 0.001 mg/kg to about 1000 mg/kg of doxycycline for at least 1 day per week and water only for the remaining days for at least 2 weeks. In some cases, the subject is administered a dosage of at least about 0.001 mg/kg to about 1000 mg/kg of doxycycline for at least 1 day per week and water only for the remaining days for at least 8 weeks. In some cases, the subject is administered a dosage of at least about 0.001 mg/kg to about 1000 mg/kg of doxycycline for at least 1 day per week and water only for the remaining days for at least 12 weeks. In some cases, the subject is administered a dosage of at least about 0.001 mg/kg to about 1000 mg/kg of doxycycline for at least 1 day per week and water only for the remaining days for at least 26 weeks. In some cases, the subject is administered a dosage of at least about 0.001 mg/kg to about 1000 mg/kg of doxycycline for at least 1 day per week and water only for the remaining days for at least 52 weeks. In some cases, the cell has increased CpG promoter hypermethylation compared to baseline. In some cases, the cell has increased telomere length compared to baseline. In some cases, the cell has from about 10% to about 50% increased median telomere length compared to baseline. In some cases, the cell has about 30% increased median telomere length compared to baseline. In some cases, the cell has from about 10% to about 50% increased 20^{th} percentile telomere length compared to baseline. In some cases, the cell has about 30% increased 20^{th} percentile telomere length compared to baseline. In some cases, the cell has about 30% increased 20^{th} percentile telomere length compared to baseline. In some cases, the cell has about 10% decreased telomeres 3 Kilo base pairs (Kbp) in length compared to baseline. In some cases, the method increases the density of anagen follicles in a subject. In some cases, the method increases the density of catagen follicles in a subject. In some cases, the method decreases adnexal atrophy in a subject. In some cases, the method decreases mononuclear perivascular inflammatory infiltrate in the dermis in a subject. In some cases, the polynucleotide and PEI is administered in a N:P ratio. In some cases, the N:P ratio is from about 1:1 to about 10:1. In some cases, the N:P ratio is about 1:1 to about 1:10. In some cases, the N:P ratio is about 5:1. In some cases, the method increases fertility in a subject. In some cases, the method increases lifespan, decreases hair loss in a subject by about 10%, and/or increases skin elasticity by about 10% in a subject compared to a subject that was not administered the method.

In some aspects, the disclosure further provides a formulation comprising, PEI, a polynucleotide encoding one or more heterologous polynucleotide(s) encoding an Octamer-binding transcription factor 4 (OCT4), wherein the one or more polynucleotide(s) encoding the OCT4 is operably linked to a promoter that is not in nature associated with the one or more polynucleotide(s) encoding the OCT4, said promoter directly or indirectly induced; one or more heterologous polynucleotide(s) encoding a Kruppel Like Factor 4 (KLF4), wherein the one or more polynucleotide(s) encoding the KLF4 is operably linked to a promoter that is not in nature associated with the one or more polynucleotide(s) encoding the KLF4, said promoter directly or indirectly induced; one or more heterologous polynucleotide(s) encoding an SRY-box 2 (SOX2), wherein the one or more polynucleotide(s) encoding the SOX2 is operably linked to an inducible promoter that is not in nature associated with the one or more polynucleotide(s) encoding the SOX2, said promoter directly or indirectly induced; wherein the polynucleotide expresses the polynucleotide(s) encoding OCT4 under conditions that induce the promoter operably linked to the polynucleotide(s) encoding OCT4; the polynucleotide(s) encoding KLF4 under conditions that induce the promoter operably linked to the polynucleotide(s) encoding KLF4; the polynucleotide(s) encoding SOX2 under conditions that include the promoter operably linked to the polynucleotide(s) encoding SOX2, and a pharmaceutically acceptable carrier. In some cases, the promoter operably linked to the polynucleotide(s) encoding OCT4, the promoter operably linked to KLF4, and the promoter operably linked to the polynucleotide(s) encoding SOX2 are the same promoter. In some cases, at least one polynucleotide is operably linked to a directly or indirectly inducible promoter that is induced by a chemical and/or nutritional inducer. In some cases, the chemical and/or nutritional inducer is selected from arabinose, IPTG, tetracycline, and rhamnose. In some cases, at least one polynucleotide is under the control of a Tet promoter sequence and a Tet repressor polynucleotide. In some cases, the OCT4 polynucleotide shares at least 80%, at least 85%, at least 90%, at least 95%, at least 98%, at least 99%, or 100% identity to SEQ ID NO: 1. In some cases, the polynucleotide encodes a OCT4 polypeptide shares at least 80%, at least 85%, at least 90%, at least 95%, at least 98%, at least 99%, or 100% identity to SEQ ID NO: 4. In some cases, the SOX2 polynucleotide shares at least 80%, at least 85%, at least 90%, at least 95%, at least 98%, at least 99%, or 100% identity to SEQ ID NO: 3. In some cases, the polynucleotide encodes a SOX2 polypeptide shares at least 80%, at least 85%, at least 90%, at least 95%, at least 98%, at least 99%, or 100% identity to SEQ ID NO: 6. In some cases, the KLF4 polynucleotide shares at least 80%, at least 85%, at least 90%, at least 95%, at least 98%, at least 99%, or 100% identity to SEQ ID NO: 2. In some cases, the polynucleotide encodes a KLF4 polypeptide shares at least 80%, at least 85%, at least 90%, at least 95%, at least 98%, at least 99%, or 100% identity to SEQ ID NO: 5. In some cases, the formulation contacts a cell of a subject. In some cases, the subject is a human. In some cases, the cell is a non-human primate cell. In some cases, the cell is a human cell. In some cases, the PEI formulation and polynucleotide are administered systemically. In some cases, the PEI formulation and polynucleotide are administered intravenously. In some cases, the PEI formulation and polynucleotide are administered in a single dose. In some cases, the PEI formulation and polynucleotide are administered in multiple doses. In some cases, the subject is administered a dosage of at least about 0.001 mg/kg to about 1000 mg/kg of doxycycline for at least 1 day per week and water only for the remaining days for at least 2 weeks. In some cases, the subject is administered a dosage of at least about 0.001 mg/kg to about 1000 mg/kg of doxycycline for at least 1 day per week and water only for the remaining days for at least 8 weeks. In some cases, the subject is administered a dosage of at least about 0.001 mg/kg to about 1000 mg/kg of doxycycline for at least 1 day per week and water only for the remaining days for at least 12 weeks. In some cases, the subject is administered a dosage of at least about 0.001 mg/kg to about 1000 mg/kg of doxycycline for at least 1 day per week and water only for the remaining days for at least 26 weeks. In some cases, the subject is administered a dosage of at least about 0.001 mg/kg to about 1000 mg/kg of doxycycline for at least 1 day per week and water only for the remaining days for at least 52 weeks. In some cases, the cell has increased CpG promoter hypermethylation compared to baseline. In some cases, the cell has increased telomere length compared to baseline. In some cases, the cell has from about 10% to about 50% increased median telomere length compared to baseline. In some cases, the cell has about 30% increased median telomere length compared to baseline. In some cases, the cell has from about 10% to about 50% increased 20^{th} percentile telomere length compared to baseline. In some cases, the cell has about 30% increased 20^{th} percentile telomere length compared to baseline. In some cases, the cell has about 30% increased 20^{th} percentile telomere length compared to baseline. In some cases, the cell has about 10% decreased telomeres 3 Kilo base pairs (Kbp) in length compared to baseline. In some cases, the formulation increases the density of anagen follicles in a subject. In some cases, the formulation increases the density of catagen follicles in a subject. In some cases, the formulation decreases adnexal atrophy in a subject. In some cases, the formulation decreases mononuclear perivascular inflammatory infiltrate in the dermis in a subject. In some cases, the polynucleotide and PEI is administered in a N:P ratio. In some cases, the N:P ratio is from about 1:1 to about 10:1. In some cases, the N:P ratio is about 1:1 to about 1:10. In some cases, the N:P ratio is about 5:1. In some cases, the method increases fertility in a subject. In some cases, the method increases lifespan, decreases hair loss in a subject by about 10%, and/or increases skin elasticity by about 10% in a subject compared to a subject that was not administered the formulation.

In some aspects, the disclosure further provides a cell comprising a polynucleotide, said polynucleotide encoding one or more heterologous polynucleotide(s) encoding one or more Yamanaka Factors, wherein the one or more polynucleotide(s) encoding the one or more Yamanaka Factors is operably linked to a promoter that is not in nature associated with the one or more polynucleotide(s) encoding the one or more Yamanaka Factors, said promoter directly or indirectly induced. In some cases, the polynucleotide expresses the polynucleotide(s) encoding one or more Yamanaka Factors under conditions that induce the promoter operably linked to the polynucleotide(s) encoding the one or more Yamanaka Factors. In some cases, the one or more heterologous polynucleotide(s) encoding one or more Yamanaka Factors comprises one or more heterologous polynucleotide(s) encoding an octamer-binding transcription factor 4 (OCT4), wherein the one or more polynucleotide(s) encoding the OCT4 is operably linked to an inducible promoter that is not in nature associated with the one or more polynucleotide(s) encoding the OCT4, said promoter directly or indirectly induced; one or more heterologous polynucleotide(s) encoding a Kruppel Like Factor 4 (KLF4), wherein the one or more polynucleotide(s) encoding the KLF4 is operably linked to a promoter that is not in nature associated with the one or more polynucleotide(s) encoding the KLF4, said promoter directly or indirectly induced; one or more heterologous polynucleotide(s) encoding an SRY-box 2 (SOX2), wherein the one or more polynucleotide(s) encoding the SOX2 is operably linked to an inducible promoter that is not in nature associated with the one or more polynucleotide(s) encoding the SOX2, said promoter directly or indirectly induced. In some cases, the polynucleotide expresses: the polynucleotide(s) encoding OCT4 under conditions that induce the promoter operably linked to the polynucleotide(s) encoding OCT4; the polynucleotide(s) encoding KLF4 under conditions that induce the promoter operably linked to the polynucleotide(s) encoding KLF4; the polynucleotide(s) encoding SOX2 under conditions that include the promoter operably linked to the polynucleotide(s) encoding SOX2. In some cases, the promoter operably linked to the polynucleotide(s) encoding OCT4, the promoter operably linked to KLF4, and the promoter operably linked to the polynucleotide(s) encoding SOX2 are the same promoter. In some cases, at least one polynucleotide is operably linked to a directly or indirectly inducible promoter that is induced by a chemical and/or nutritional inducer. In some cases, the chemical and/or nutritional inducer is selected from arabinose, IPTG, tetracycline, and rhamnose. In some cases, at least one polynucleotide is under the control of a Tet promoter sequence and a Tet repressor polynucleotide. In some cases, the OCT4 polynucleotide shares at least 80%, at least 85%, at least 90%, at least 95%, at least 98%, at least 99%, or 100% identity to SEQ ID NO: 1. In some cases, the polynucleotide encodes a OCT4 polypeptide shares at least 80%, at least 85%, at least 90%, at least 95%, at least 98%, at least 99%, or 100% identity to SEQ ID NO: 4. In some cases, the SOX2 polynucleotide shares at least 80%, at least 85%, at least 90%, at least 95%, at least 98%, at least 99%, or 100% identity to SEQ ID NO: 3. In some cases, the polynucleotide encodes a SOX2 polypeptide shares at least 80%, at least 85%, at least 90%, at least 95%, at least 98%, at least 99%, or 100% identity to SEQ ID NO: 6. In some cases, the KLF4 polynucleotide shares at least 80%, at least 85%, at least 90%, at least 95%, at least 98%, at least 99%, or 100% identity to SEQ ID NO: 2. In some cases, the polynucleotide encodes a KLF4 polypeptide shares at least 80%, at least 85%, at least 90%, at least 95%, at least 98%, at least 99%, or 100% identity to SEQ ID NO: 5. In some cases, the cell is a non-human primate cell. In some cases, the cell is a human cell. In some cases, doxycycline is administered to the cell. In some cases, the cell is administered a dosage of at least about 0.001 mg/kg to about 1000 mg/kg of doxycycline for at least 1 day per week for at least 2 weeks. In some cases, the cell is administered a dosage of at least about 0.001 mg/kg to about 1000 mg/kg of doxycycline for at least 1 day per week for at least 8 weeks. In some cases, the cell is administered a dosage of at least about 0.001 mg/kg to about 1000 mg/kg of doxycycline for at least 1 day per week for at least 12 weeks. In some cases, the cell is administered a dosage of at least about 0.001 mg/kg to about 1000 mg/kg of doxycycline for at least 1 day per week for at least 26 weeks. In some cases, the cell is administered a dosage of at least about 0.001 mg/kg to about 1000 mg/kg of doxycycline for at least 1 day per week for at least 52 weeks. In some cases, the cell has increased CpG promoter hypermethylation compared to a cell that does not comprise the polynucleotide. In some cases, the cell has increased telomere length compared to a cell that does not comprise the polynucleotide. In some cases, the cell has from about 10% to about 50% increased median telomere length compared to a cell that does not comprise the polynucleotide. In some cases, the cell has about 30% increased median telomere length compared to a cell that does not comprise the polynucleotide. In some cases, the cell has from about 10% to about 50% increased 20^{th} percentile telomere length compared to a cell that does not comprise the polynucleotide. In some cases, the cell has about 30% increased 20^{th} percentile telomere length compared to a cell that does not comprise the polynucleotide. In some cases, the cell has about 30% increased 20^{th} percentile telomere length compared to baseline. In some cases, the cell has about 10% decreased telomeres 3 Kilo base pairs (Kbp) in length compared to a cell that does not comprise the polynucleotide. In some cases, the cell has an increased lifespan compared to a cell that was not comprise the polynucleotide.

### BRIEF DESCRIPTION OF THE FIGURES

FIG. 1A represents fluorescent images of 10 organs from mouse 1 injected with AAV9-GFP.

FIG. 1B represents fluorescent images of 10 organs from mouse 1 injected with AAV9-RFP.

FIG. 1C represents fluorescent images of 10 organs from mouse 2 injected with AAV9-GFP.

FIG. 1D represents fluorescent images of 10 organs from mouse 2 injected with AAV9-RFP.

FIG. 1E represents fluorescent images of 10 organs from mouse 3 injected with AAV9-GFP.

FIG. 1F represents fluorescent images of 10 organs from mouse 3 injected with AAV9-RFP.

FIG. 1G represents fluorescent images of 10 organs from mouse 4 injected with AAV9-GFP.

FIG. 1H represents fluorescent images of 10 organs from mouse 4 injected with AAV9-RFP.

FIG. 1I represents fluorescent images from bone from mice 1-4 after injection with AAV9-GFP.

FIG. 1J represents fluorescent images from bone from mice 1-4 after injection with AAV9-RFP.

FIG. 1K represents fluorescent images from brain from mice 1-4 after injection with AAV9-GFP.

FIG. 1L represents fluorescent images from brain from mice 1-4 after injection with AAV9-RFP.

FIG. 1M represents fluorescent images from colon from mice 1-4 after injection with AAV9-GFP.

FIG. 1N represents fluorescent images from colon from mice 1-4 after injection with AAV9-RFP.

FIG. 1O represents fluorescent images from kidney from mice 1-4 after injection with AAV9-GFP.

FIG. 1P represents fluorescent images from kidney from mice 1-4 after injection with AAV9-RFP.

FIG. 1Q represents fluorescent images from liver from mice 1-4 after injection with AAV9-GFP.

FIG. 1R represents fluorescent images from liver from mice 1-4 after injection with AAV9-RFP.

FIG. 1S represents fluorescent images from lung from mice 1-4 after injection with AAV9-GFP.

FIG. 1T represents fluorescent images from lung from mice 1-4 after injection with AAV9-RFP.

FIG. 1U represents fluorescent images from muscle from mice 1-4 after injection with AAV9-GFP.

FIG. 1V represents fluorescent images from muscle from mice 1-4 after injection with AAV9-RFP.

FIG. 1W represents fluorescent images from skin from mice 1-4 after injection with AAV9-GFP.

FIG. 1X represents fluorescent images from skin from mice 1-4 after injection with AAV9-RFP.

FIG. 1Y represents fluorescent images from spleen from mice 1-4 after injection with AAV9-GFP.

FIG. 1Z represents fluorescent images from spleen from mice 1-4 after injection with AAV9-RFP.

FIG. 2A represents fluorescent images of 10 organs from a mouse injected with saline (negative control).

FIG. 2B represents fluorescent images of 10 organs from a mouse injected with 0.4×10¹³ plasmids (group 2).

FIG. 2C represents fluorescent images of 10 organs from a mouse injected with 0.2×10¹⁴ plasmids (group 3).

FIG. 2D represents fluorescent images of 10 organs from a mouse injected with 0.2×10¹⁵ plasmids (group 3).

FIG. 2E represents fluorescent images of 10 organs from a mouse injected with 0.3×10¹⁴ plasmids (group 4).

FIG. 2F represents fluorescent images of 10 organs from a mouse injected with 0.3×10¹⁴ plasmids (group 4).

FIG. 2G represents fluorescent images of 10 organs from a mouse injected with 0.4×10¹⁴ plasmids (group 5).

FIG. 211 represents the mean GFP density from the fluorescent images of the 10 organs.

FIG. 3A shows a map of the hOSK plasmid.

FIG. 3B shows in vitro expression of OCT4 expressed from the hOSK plasmid for 4 controls and 6 samples transfected with the OSK plasmid.

FIG. 3C shows in vitro expression of SOX2 expressed from the hOSK plasmid for 4 controls and 6 samples transfected with the OSK plasmid.

FIG. 3D shows in vitro expression of KLF4 expressed from the hOSK plasmid for 4 controls and 6 samples transfected with the OSK plasmid.

FIG. 4A is a hematoxylin and eosin (H&E) stained NHP skin section at 20× showing marked adnexal atrophy and minimal perivascular mononuclear inflammatory cell infiltrate for sample S1-1. Catagen and anagen follicles are marked by an arrow and arrowhead, respectively.

FIG. 4B is a hematoxylin and eosin (H&E) stained NHP skin section at 20× showing marked adnexal atrophy and minimal perivascular mononuclear inflammatory cell infiltrate for sample S2-1. A telogen follicle is marked by an arrow.

FIG. 4C is a hematoxylin and eosin (H&E) stained NHP skin section at 20× showing moderate adnexal atrophy and minimal perivascular mononuclear inflammatory cell infiltrate, along with catagen and telogen follicles for sample S3-1.

FIG. 4D is a hematoxylin and eosin (H&E) stained NHP skin section at 20× showing mild adnexal atrophy and minimal perivascular mononuclear inflammatory cell infiltrate, along with catagen and telogen follicles for sample S4-1.

FIG. 4E is a hematoxylin and eosin (H&E) stained NHP skin section at 20× showing minimal perivascular mononuclear inflammatory cell infiltrate, along with anagen follicles for sample S1-2.

FIG. 4F is a hematoxylin and eosin (H&E) stained NHP skin section at 20× showing minimal perivascular mononuclear inflammatory cell infiltrate, along with anagen follicles for sample S2-2.

FIG. 4G is a hematoxylin and eosin (H&E) stained NHP skin section at 20× showing mild adnexal atrophy, minimal perivascular mononuclear inflammatory cell infiltrate, along with anagen follicles for sample S3-2.

FIG. 411 is a hematoxylin and eosin (H&E) stained NHP skin section at 20× showing minimal perivascular mononuclear inflammatory cell infiltrate, along with anagen follicles for sample S4-2.

FIG. 41 is a hematoxylin and eosin (H&E) stained NHP skin section at 20× showing marked adnexal atrophy and minimal perivascular mononuclear inflammatory cell infiltrate, as well as telogen follicles, for sample S1-3.

FIG. 4J is a hematoxylin and eosin (H&E) stained NHP skin section at 20× showing marked adnexal atrophy and minimal perivascular mononuclear inflammatory cell infiltrate, as well as telogen follicles, for sample S2-3.

FIG. 4K is a hematoxylin and eosin (H&E) stained NHP skin section at 20× showing moderate adnexal atrophy and minimal perivascular mononuclear inflammatory cell infiltrate, as well as anagen follicles, for sample S3-3.

FIG. 4L is a hematoxylin and eosin (H&E) stained NHP skin section at 20× showing mild adnexal atrophy and minimal perivascular mononuclear inflammatory cell infiltrate, as well as anagen, catagen, and telogen follicles, for sample S4-3.

FIG. 5A shows a graph that compares the median telomere before (BS) and after (AS) in vivo induction of Yamanaka Factors for each NHP (S1, S2, S3 and S4).

FIG. 5B shows a graph that compares the median 20^{th} percentile telomere length before (BS) and after (AS) in vivo induction of Yamanaka Factors for each NHP (S1, S2, S3 and S4).

FIG. 5C shows a graph that compares the percent of telomeres that are less than 3 kilo base pairs before (BS) and after (AS) in vivo induction of Yamanaka Factors for each NHP (S1, S2, S3 and S4).

FIG. 5D shows a graph that compares the telomere length percentiles before (BS) and after (AS) in vivo induction of Yamanaka Factors for each NHP (S1, S2, S3 and S4).

FIG. 6A shows a graph that compares the median telomere after delivery of AAV9-GFP or various concentrations of AAV9-TERT in NHP.

FIG. 6B shows a graph that compares the median 20^{th} percentile telomere length after delivery of AAV9-GFP or various concentrations of AAV9-TERT in NHP.

FIG. 6C shows a graph that compares the percent of telomeres that are less than 3 kilo base pairs after delivery of AAV9-GFP or various concentrations of AAV9-TERT in NHP.

### DETAILED DESCRIPTION

### Definitions

Unless defined otherwise herein, all technical and scientific terms used herein shall have the meanings that are commonly understood by those of ordinary skill in the art to which the present disclosure belongs.

The term “comprising” is intended to mean that the compositions and methods include the recited elements, but do not exclude others. Consisting essentially of when used to define compositions and methods, shall mean excluding other elements of any essential significance to the combination when used for the intended purpose. Thus, a composition consisting essentially of the elements as defined herein would not exclude trace contaminants or inert carriers. “Consisting of” shall mean excluding more than trace elements of other ingredients and substantial method steps. Embodiments defined by each of these transition terms are within the scope of this disclosure.

The term “composition” is also intended to encompass a combination of active agent and another carrier, e.g., compound or composition, inert (for example, a detectable agent or label) or active, such as an adjuvant, diluent, binder, stabilizer, buffers, salts, lipophilic solvents, preservative, adjuvant or the like. Carriers also include pharmaceutical excipients and additives proteins, peptides, amino acids, lipids, and carbohydrates (e.g., sugars, including monosaccharides, di-, tri-, tetra-, and oligosaccharides; derivatized sugars such as alditols, aldonic acids, esterified sugars and the like; and polysaccharides or sugar polymers), which can be present singly or in combination, comprising alone or in combination 1-99.99% by weight or volume. Exemplary protein excipients include serum albumin such as human serum albumin (HSA), recombinant human albumin (rHA), gelatin, casein, and the like. Representative amino acid/antibody components, which can also function in a buffering capacity, include alanine, glycine, arginine, betaine, histidine, glutamic acid, aspartic acid, cysteine, lysine, leucine, isoleucine, valine, methionine, phenylalanine, aspartame, and the like. Carbohydrate excipients are also intended within the scope of this disclosure, examples of which include but are not limited to monosaccharides such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol sorbitol (glucitol) and myoinositol.

The term “pharmaceutically acceptable carrier” (or medium), which may be used interchangeably with the term biologically compatible carrier or medium, refers to reagents, cells, compounds, materials, compositions, and/or dosage forms that are not only compatible with the cells and other agents to be administered therapeutically, but also are, within the scope of sound medical judgment, suitable for use in contact with the tissues of human beings and animals without excessive toxicity, irritation, allergic response, or other complication commensurate with a reasonable benefit/risk ratio. Pharmaceutically acceptable carriers suitable for use in the present disclosure include liquids, semi-solid (e.g., gels) and solid materials (e.g., cell scaffolds and matrices, tubes sheets and other such materials as known in the art and described in greater detail herein). These semi-solid and solid materials may be designed to resist degradation within the body (non-biodegradable) or they may be designed to degrade within the body (biodegradable, bioerodable). A biodegradable material may further be bioresorbable or bioabsorbable, i.e., it may be dissolved and absorbed into bodily fluids (water-soluble implants are one example), or degraded and ultimately eliminated from the body, either by conversion into other materials or breakdown and elimination through natural pathways.

The term “excipient” refers to an inert substance added to a pharmaceutical composition to further facilitate administration of an active ingredient. Examples include, but are not limited to, calcium bicarbonate, calcium phosphate, various sugars and types of starch, cellulose derivatives, gelatin, vegetable oils, polyethylene glycols, and surfactants, including, for example, polysorbate 20.

As used herein, the term “subject” refers to a cell, an animal, or a mammal. In some embodiments, the cell is a mammalian cell. For the purpose of illustration only, a mammal includes but is not limited to a human, a feline, a canine, a simian, a murine, a bovine, an equine, a porcine or an ovine.

The term “effective amount” refers to an amount that can achieve an indicated result. For example, an “effective amount” of transcription factors enhances muscle strength, cognition, or prevents or reduces loss of mobility in an aging subject.

The term “therapeutically effective amount” refers to an amount effective for lessening, ameliorating, eliminating, preventing, or inhibiting at least one symptom associated with aging and may be empirically determined. In some embodiments, a “therapeutically effective amount” is a muscle strength promoting amount, a cognition promoting amount, a mobility promoting amount, and/or an amount sufficient to achieve a statistically significant promotion muscle strength, cognition, or mobility compared to a control.

In some embodiments, the method includes administering an effective amount of the transcription factors, or a composition which includes an effective amount of transcription factors, to the subject.

The transcription factors, transcription factors compositions, or other therapeutic agents of the present disclosure can be formulated into therapeutically active pharmaceutical compositions that can be administered to a subject parenterally or orally. Parenteral administration routes include, but are not limited to epidermal, intraarterial, intramuscular (IM and depot IM), intraperitoneal (IP), intravenous (IV), intrasternal injection or infusion techniques, intranasal (inhalation), intrathecal, injection into the stomach, subcutaneous injections, and transdermal injections.

In some embodiments, the effective amount of transcription factors or transcription factors composition is administered as a single dose per time period, such as every three or four months, month, week, or day, or it can be divided into at least two-unit dosages for administration over a period. Treatment may be continued as long as necessary to achieve the desired results. For instance, treatment may continue for about 3 or 4 weeks up to about 12-24 months or longer, including ongoing treatment. The transcription factors can also be administered in several doses intermittently, such as every few days (for example, at least about every two, three, four, five, or ten days) or every few weeks (for example at least about every two, three, four, five, or ten weeks).

As used herein, the term “promoter” refers to a nucleic acid sequence sufficient to direct transcription of a gene. Also included in the disclosure are those promoter elements which are sufficient to render promoter dependent gene expression controllable for cell type specific, tissue specific or inducible by external signals or agents.

The term “constitutive promoter” refers to a promoter capable of directing transcription of an operably linked nucleic acid molecule in the absence of a stimulus (e.g., heat shock, chemicals, light, etc.).

The term “inducible promoter” refers to a regulatory region that is operably linked to one or more genes, wherein expression of the gene(s) is increased in the presence of an inducer of said regulatory region.

In some embodiments, a promoter is an inducible promoter or a discrete promoter. Inducible promoters can be turned on by a chemical or a physical condition such as temperature or light. Examples of chemical promoters include, without limitation, alcohol-regulated, tetracycline-regulated, steroid-regulated, metal-regulated and pathogenesis-related promoters. Examples of discrete promoters can be found in, for examples, Wolfe et al., (2002) Mol. Endocrinol. 16:435-449.

The term “operably linked” refers to the association of nucleic acid sequences on a single nucleic acid fragment so that the function of one is affected by the other. A regulatory element is operably linked with a coding sequence when it is capable of affecting the expression of the gene coding sequence, regardless of the distance between the regulatory element and the coding sequence. More specifically, operably linked refers to a nucleic acid sequence that is joined to a regulatory sequence in a manner which allows expression of the nucleic acid sequence. In other words, the regulatory sequence acts in cis. In one embodiment, a gene may be “directly linked” to a regulatory sequence in a manner which allows expression of the gene. In another embodiment, a gene may be “indirectly linked” to a regulatory sequence in a manner which allows expression of the gene. In one embodiment, two or more genes may be directly or indirectly linked to a regulatory sequence in a manner which allows expression of the two or more genes.

The term “polynucleotide” refers to deoxyribonucleic acid (DNA), ribonucleic acid (RNA) or analogs thereof. The polynucleotide can be single-stranded, double-stranded, or contain both single-stranded and double-stranded sequences.

The term “polypeptide” includes “polypeptide” as well as “polypeptides,” and refers to a molecule composed of amino acid monomers linearly linked by amide bonds (i.e., peptide bonds). The term “polypeptide” also refers to any chain or chains of two or more amino acids and does not refer to a specific length of the product. Thus, “peptides,” “dipeptides,” “tripeptides,” “oligopeptides,” “protein,” “amino acid chain,” or any other term used to refer to a chain or chains of two or more amino acids, are included within the definition of “polypeptide,” and the term “polypeptide” may be used instead of, or interchangeably with any of these terms. The term “dipeptide” refers to a peptide of two linked amino acids. The term “tripeptide” refers to a peptide of three linked amino acids. The term “polypeptide” is also intended to refer to the products of post-expression modifications of the polypeptide, including but not limited to glycosylation, acetylation, phosphorylation, amidation, derivatization, proteolytic cleavage, or modification by non-naturally occurring amino acids. A polypeptide may be derived from a natural biological source or produced by recombinant technology. A polypeptide of the disclosure may be of a size of about 3 or more, 5 or more, 10 or more, 20 or more, 25 or more, 50 or more, 75 or more, 100 or more, 200 or more, 500 or more, 1,000 or more, or 2,000 or more amino acids. Polypeptides may have a defined three-dimensional structure, although they do not necessarily have such structure. Polypeptides with a defined three-dimensional structure are referred to as folded, and polypeptides, which do not possess a defined three-dimensional structure, but rather can adopt a large number of different conformations, are referred to as unfolded. The term “peptide” or “polypeptide” may refer to an amino acid sequence that corresponds to a protein or a portion of a protein or may refer to an amino acid sequence that corresponds with non-protein sequence, e.g., a sequence selected from a regulatory peptide sequence, leader peptide sequence, signal peptide sequence, linker peptide sequence, and other peptide sequence.

The term “transcription factor” as used herein means a protein that possesses a biological function including regulation of transcription of genes. That is, a transcription factor is a protein that possesses a DNA-binding domain (DBD) that allows the protein to bind a specific sequence of DNA (an enhancer element or promoter sequence). Upon binding the enhancer or promoter element, the transcription factor's presence can aide in initiation of transcription by stabilizing transcription initiation complex formation and/or activity, for example. Transcription factors also bind to regulatory DNA sequences, such as enhancer sequences, that can be many hundreds of base pairs downstream or upstream from the transcribed gene. Transcription factors are also referred to as sequence-specific DNA-binding factors. Transcription factors can perform the transcription controlling function either alone or in combination with other proteins, i.e. by forming an activation complex, and can aide in recruiting RNA polymerase and related proteins to the transcription initiation start site. (See, Nevins, Science 258, 424-429 (1992); Dalton, EMBO J. 11, 11797 (1992); Yee et al. ibid. 6, 2061 (1987), Weintraub et al., Nature 358, 259-261 (1992), Pagano et al., Science 255, 1144-1147 (1992)). For a complete list of known eukaryotic transcription factors, see Fulton et al., Genome Biology, 10:R29, 2009 “TFCat: the curated catalog of mouse and human transcription factors.” Examples of transcription factors include, but are not limited to, Yamanaka factors (Oct3/4, Sox2, Kl4, and c-Myc), T-box 21 (T-bet), T-box 1, T-Brain 1, T-box 2, T-box 6, signal transducer and activator of transcription 1 (STAT1), STAT2, STAT3, STAT4, STAT5, STAT6, Runx1, Runx3, and Eomesodermin, for example.

As used herein, sequence “identity” may be determined by using the stand-alone executable BLAST engine program for blasting two sequences (bl2seq), which can be retrieved from the National Center for Biotechnology Information (NCBI) ftp site, using the default parameters (Tatusova and Madden, FEMS Microbiol Lett., 1999, 174, 247-250; which is incorporated herein by reference in its entirety). The terms “identical” or “identity” when used in the context of two or more nucleic acids or polypeptide sequences, refer to the number or percentage of residues that are the same in a sequence of interest and a reference sequence. The percentage can be calculated by optimally aligning the sequence of interest to the reference sequence; comparing the two sequences over the entire length of the reference sequence; determining the number of positions at which the identical amino acid residue or nucleic acid base occurs in both sequences to yield the number of matched positions; dividing the number of matched positions by the total number of positions in the reference sequence adjusted by adding the number of gap positions introduced into the reference sequence in generating the alignment; and multiplying the result by 100 to yield the percentage of sequence identity. When comparing DNA and RNA, thymine (T) and uracil (U) can be considered equivalent. Identity calculation can be performed manually or by the BLAST algorithm. A sequence of interest may be at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 100% to a reference sequence.

The term “cellular longevity” refers to increasing the lifespan and/or decreasing signs of aging in a cell or organism. In some embodiments, the cell is a human cell. In some embodiments, an organism is a subject as defined herein. Signs of aging include, but are not limited to, telomere length, methylation markers, alopecia indicators, decrease in fertility, and/or clinical observations.

### Polynucleotide

In some embodiments, the polynucleotide of the disclosure comprises a gene encoding OCT4 (SEQ ID NO. 4), wherein the OCT4 gene (SEQ ID NO. 1) is operably linked to a directly or indirectly promoter.

In some embodiments, the promoter is an inducible promoter.

In some embodiments, the promoter is a constitutive promoter.

In some embodiments, the polynucleotide of the disclosure comprise a gene encoding KLF4 (SEQ ID NO. 5), wherein the KLF4 gene (SEQ ID NO. 2) is operably linked to a directly or indirectly inducible promoter.

In some embodiments, the polynucleotide of the disclosure comprise a gene encoding SOX2 (SEQ ID NO. 6), wherein the SOX2 gene (SEQ ID NO. 3) is operably linked to a directly or indirectly inducible promoter.

In some embodiments, the promoter that is operably linked to OCT4, KLF4, and/or SOX2 is directly or indirectly induced by exogenous conditions. In some embodiments, the promoter is directly or indirectly induced a chemical signal.

| Gene | Sequence | SEQ ID NO. |
|---|---|---|
| OCT4 | ATGGCGGGACACCTGGCTTCGGATTTCGCCTTCTCGCCCC | 1 |
| | CTCCAGGTGGTGGAGGTGATGGGCCAGGGGGGCCGGAGC | |
| | CGGGCTGGGTTGATCCTCGGACCTGGCTAAGCTTCCAAGG | |
| | CCCTCCTGGAGGGCCAGGAATCGGGCCGGGGGTTGGGCC | |
| | AGGCTCTGAGGTGTGGGGGATTCCCCCATGCCCCCCGCCG | |
| | TATGAGTTCTGTGGGGGGATGGCGTACTGTGGGCCCCAGG | |
| | TTGGAGTGGGGCTAGTGCCCCAAGGCGGCTTGGAGACCTC | |
| | TCAGCCTGAGGGTGAAGCAGGAGTCGGGGTGGAGAGCAA | |
| | CTCCGATGGGGCCTCCCCGGAGCCCTGCACCGTCACCCCT | |
| | GGTGCCGTGAAGCTGGAGAAGGAGAAGCTGGAGCAAAAC | |
| | CCGGAGGAGTCCCAGGACATCAAAGCTCTGCAGAAAGAA | |
| | CTCGAGCAATTTGCCAAGCTCCTGAAGCAGAAGAGGATCA | |
| | CCCTGGGATATACACAGGCCGATGTGGGGCTCACCCTGGG | |
| | GGTTCTATTTGGGAAGGTATTCAGCCAAACGACCATCTGC | |
| | CGCTTTGAGGCTCTGCAGCTTAGCTTCAAGAACATGTGTA | |
| | AGCTGCGGCCCTTGCTGCAGAAGTGGGTGGAGGAAGCTG | |
| | ACAACAATGAAAATCTTCAGGAGATATGCAAAGCAGAAA | |
| | CCCTCGTGCAGGCCCGAAAGAGAAAGCGAACCAGTATCG | |
| | AGAACCGAGTGAGAGGCAACCTGGAGAATTTGTTCCTGCA | |
| | GTGCCCGAAACCCACACTGCAGCAGATCAGCCACATCGCC | |
| | CAGCAGCTTGGGCTCGAGAAGGATGTGGTCCGAGTGTGGT | |
| | TCTGTAACCGGCGCCAGAAGGGCAAGCGATCAAGCAGCG | |
| | ACTATGCACAACGAGAGGATTTTGAGGCTGCTGGGTCTCC | |
| | TTTCTCAGGGGGACCAGTGTCCTTTCCTCTGGCCCCAGGG | |
| | CCCCATTTTGGTACCCCAGGCTATGGGAGCCCTCACTTCA | |
| | CTGCACTGTACTCCTCGGTCCCTTTCCCTGAGGGGGAAGC | |
| | CTTTCCCCCTGTCTCTGTCACCACTCTGGGCTCTCCCATGC | |
| | ATTCAAAC | |
| KLF4 | ATGGCTGTCAGCGACGCGCTGCTCCCATCTTTCTCCACGTT | 2 |
| | CGCGTCTGGCCCGGCGGGAAGGGAGAAGACACTGCGTCA | |
| | AGCAGGTGCCCCGAATAACCGCTGGCGGGAGGAGCTCTC | |
| | CCACATGAAGCGACTTCCCCCAGTGCTTCCCGGCCGCCCC | |
| | TATGACCTGGCGGCGGCGACCGTGGCCACAGACCTGGAG | |
| | AGCGGCGGAGCCGGTGCGGCTTGCGGCGGTAGCAACCTG | |
| | GCGCCCCTACCTCGGAGAGAGACCGAGGAGTTCAACGAT | |
| | CTCCTGGACCTGGACTTTATTCTCTCCAATTCGCTGACCCA | |
| | TCCTCCGGAGTCAGTGGCCGCCACCGTGTCCTCGTCAGCG | |
| | TCAGCCTCCTCTTCGTCGTCGCCGTCGAGCAGCGGCCCTG | |
| | CCAGCGCGCCCTCCACCTGCAGCTTCACCTATCCGATCCG | |
| | GGCCGGGAACGACCCGGGCGTGGCGCCGGGCGGCACGGG | |
| | CGGAGGCCTCCTCTATGGCAGGGAGTCCGCTCCCCCTCCG | |
| | ACGGCTCCCTTCAACCTGGCGGACATCAACGACGTGAGCC | |
| | CCTCGGGCGGCTTCGTGGCCGAGCTCCTGCGGCCAGAATT | |
| | GGACCCGGTGTACATTCCGCCGCAGCAGCCGCAGCCGCCA | |
| | GGTGGCGGGCTGATGGGCAAGTTCGTGCTGAAGGCGTCGC | |
| | TGAGCGCCCCTGGCAGCGAGTACGGCAGCCCGTCGGTCAT | |
| | CAGCGTCAGCAAAGGCAGCCCTGACGGCAGCCACCCGGT | |
| | GGTGGTGGCGCCCTACAACGGCGGGCCGCCGCGCACGTG | |
| | CCCCAAGATCAAGCAGGAGGCGGTCTCTTCGTGCACCCAC | |
| | TTGGGCGCTGGACCCCCTCTCAGCAATGGCCACCGGCCGG | |
| | CTGCACACGACTTCCCCCTGGGGCGGCAGCTCCCCAGCAG | |
| | GACTACCCCGACCCTGGGTCTTGAGGAAGTGCTGAGCAGC | |
| | AGGGACTGTCACCCTGCCCTGCCGCTTCCTCCCGGCTTCCA | |
| | TCCCCACCCGGGGCCCAATTACCCATCCTTCCTGCCCGATC | |
| | AGATGCAGCCGCAAGTCCCGCCGCTCCATTACCAAGAGCT | |
| | CATGCCACCCGGTTCCTGCATGCCAGAGGAGCCCAAGCCA | |
| | AAGAGGGGAAGACGATCGTGGCCCCGGAAAAGGACCGCC | |
| | ACCCACACTTGTGATTACGCGGGCTGCGGCAAAACCTACA | |
| | CAAAGAGTTCCCATCTCAAGGCACACCTGCGAACCCACAC | |
| | AGGTGAGAAACCTTACCACTGTGACTGGGACGGCTGTGGA | |
| | TGGAAATTCGCCCGCTCAGATGAACTGACCAGGCACTACC | |
| | GTAAACACACGGGGCACCGCCCGTTCCAGTGCCAAAAAT | |
| | GCGACCGAGCATTTTCCAGGTCGGACCACCTCGCCTTACA | |
| | CATGAAGAGGCATTTT | |
| SOX2 | ATGTACAACATGATGGAGACGGAGCTGAAGCCGCCGGGC | 3 |
| | CCGCAGCAAACTTCGGGGGGCGGCGGCGGCAACTCCACC | |
| | GCGGCGGCGGCCGGCGGCAACCAGAAAAACAGCCCGGAC | |
| | CGCGTCAAGCGGCCCATGAATGCCTTCATGGTGTGGTCCC | |
| | GCGGGCAGCGGCGCAAGATGGCCCAGGAGAACCCCAAGA | |
| | TGCACAACTCGGAGATCAGCAAGCGCCTGGGCGCCGAGT | |
| | GGAAACTTTTGTCGGAGACGGAGAAGCGGCCGTTCATCGA | |
| | CGAGGCTAAGCGGCTGCGAGCGCTGCACATGAAGGAGCA | |
| | CCCGGATTATAAATACCGGCCCCGGCGGAAAACCAAGAC | |
| | GCTCATGAAGAAGGATAAGTACACGCTGCCCGGCGGGCT | |
| | GCTGGCCCCCGGCGGCAATAGCATGGCGAGCGGGGTCGG | |
| | GGTGGGCGCCGGCCTGGGCGCGGGCGTGAACCAGCGCAT | |
| | GGACAGTTACGCGCACATGAACGGCTGGAGCAACGGCAG | |
| | CTACAGCATGATGCAGGACCAGCTGGGCTACCCGCAGCAC | |
| | CCGGGCCTCAATGCGCACGGCGCAGCGCAGATGCAGCCC | |
| | ATGCACCGCTACGACGTGAGCGCCCTGCAGTACAACTCCA | |
| | TGACCAGCTCGCAGACCTACATGAACGGCTCGCCCACCTA | |
| | CAGCATGTCCTACTCGCAGCAGGGCACCCCTGGCATGGCT | |
| | CTTGGCTCCATGGGTTCGGTGGTCAAGTCCGAGGCCAGCT | |
| | CCAGCCCCCCTGTGGTTACCTCTTCCTCCCACTCCAGGGCG | |
| | CCCTGCCAGGCCGGGGACCTCCGGGACATGATCAGCATGT | |
| | ATCTCCCCGGCGCCGAGGTGCCGGAACCCGCCGCCCCCAG | |
| | CAGACTTCACATGTCCCAGCACTACCAGAGCGGCCCGGTG | |
| | CCCGGCACGGCCATTAACGGCACACTGCCCCTCTCACACA | |
| | TGTGA | |
| OCT4 | MAGHLASDFAFSPPPGGGGDGPGGPEPGWVDPRTWLSFQGP | 4 |
| | PGGPGIGPGVGPGSEVWGIPPCPPPYEFCGGMAYCGPQVGVG | |
| | LVPQGGLETSQPEGEAGVGVESNSDGASPEPCTVTPGAVKLE | |
| | KEKLEQNPEESQDIKALQKELEQFAKLLKQKRITLGYTQADV | |
| | GLTLGVLFGKVFSQTTICRFEALQLSFKNMCKLRPLLQKWVE | |
| | EADNNENLQEICKAETLVQARKRKRTSIENRVRGNLENLFLQ | |
| | CPKPTLQQISHIAQQLGLEKDVVRVWFCNRRQKGKRSSSDY | |
| | AQREDFEAAGSPFSGGPVSFPLAPGPHFGTPGYGSPHFTALYS | |
| | SVPFPEGEAFPPVSVTTLGSPMHSN | |
| KLF4 | MAVSDALLPSFSTFASGPAGREKTLRQAGAPNNRWREELSH | 5 |
| | MKRLPPVLPGRPYDLAAATVATDLESGGAGAACGGSNLAPL | |
| | PRRETEEFNDLLDLDFILSNSLTHPPESVAATVSSSASASSSSS | |
| | PSSSGPASAPSTCSFTYPIRAGNDPGVAPGGTGGGLLYGRESA | |
| | PPPTAPFNLADINDVSPSGGFVAELLRPELDPVYIPPQQPQPPG | |
| | GGLMGKFVLKASLSAPGSEYGSPSVISVSKGSPDGSHPVVVA | |
| | PYNGGPPRTCPKIKQEAVSSCTHLGAGPPLSNGHRPAAHDFP | |
| | LGRQLPSRTTPTLGLEEVLSSRDCHPALPLPPGFHPHPGPNYP | |
| | SFLPDQMQPQVPPLHYQELMPPGSCMPEEPKPKRGRRSWPR | |
| | KRTATHTCDYAGCGKTYTKSSHLKAHLRTHTGEKPYHCDW | |
| | DGCGWKFARSDELTRHYRKHTGHRPFQCQKCDRAFSRSDH | |
| | LALHMKRHF | |
| SOX2 | MYNMMETELKPPGPQQTSGGGGGNSTAAAAGGNQKNSPDR | 6 |
| | VKRPMNAFMVWSRGQRRKMAQENPKMHNSEISKRLGAEW | |
| | KLLSETEKRPFIDEAKRLRALHMKEHPDYKYRPRRKTKTLM | |
| | KKDKYTLPGGLLAPGGNSMASGVGVGAGLGAGVNQRMDS | |
| | YAHMNGWSNGSYSMMQDQLGYPQHPGLNAHGAAQMQPM | |
| | HRYDVSALQYNSMTSSQTYMNGSPTYSMSYSQQGTPGMAL | |
| | GSMGSVVKSEASSSPPVVTSSSHSRAPCQAGDLRDMISMYLP | |
| | GAEVPEPAAPSRLHMSQHYQSGPVPGTAINGTLPLSHM | |

In some embodiments, the Tet-regulated promoter sequence with associated operator sequences is inserted upstream of the OCT4, KLF4, and/or SOX2 gene in the polynucleotide and a Tet-On® 3G Transactivator gene (SEQ ID NO. 7) is inserted under the control of a constitutive promoter, e.g., the cytomegalovirus (CMV) promoter. The Tet-On® 3G transactivator protein (SEQ ID NO. 8) activates transcription from the Tet promoter (SEQ ID NO. 9) when tetracycline (or doxycycline) is present.

| Gene | Sequence | SEQ ID NO. |
|---|---|---|
| Tet-On 3G | ATGTCTAGACTGGACAAGAGCAAAGTCATAAACTCT | 7 |
| | GCTCTGGAATTACTCAATGGAGTCGGTATCGAAGGCC | |
| | TGACGACAAGGAAACTCGCTCAAAAGCTGGGAGTTG | |
| | AGCAGCCTACCCTGTACTGGCACGTGAAGAACAAGC | |
| | GGGCCCTGCTCGATGCCCTGCCAATCGAGATGCTGGA | |
| | CAGGCATCATACCCACTCCTGCCCCCTGGAAGGCGAG | |
| | TCATGGCAAGACTTTCTGCGGAACAACGCCAAGTCAT | |
| | ACCGCTGTGCTCTCCTCTCACATCGCGACGGGGCTAA | |
| | AGTGCATCTCGGCACCCGCCCAACAGAGAAACAGTA | |
| | CGAAACCCTGGAAAATCAGCTCGCGTTCCTGTGTCAG | |
| | CAAGGCTTCTCCCTGGAGAACGCACTGTACGCTCTGT | |
| | CCGCCGTGGGCCACTTTACACTGGGCTGCGTATTGGA | |
| | GGAACAGGAGCATCAAGTAGCAAAAGAGGAAAGAG | |
| | AGACACCTACCACCGATTCTATGCCCCCACTTCTGAA | |
| | ACAAGCAATTGAGCTGTTCGACCGGCAGGGAGCCGA | |
| | ACCTGCCTTCCTTTTCGGCCTGGAACTAATCATATGT | |
| | GGCCTGGAGAAACAGCTAAAGTGCGAAAGCGGCGGG | |
| | CCGACCGACGCCCTTGACGATTTTGACTTAGACATGC | |
| | TCCCAGCCGATGCCCTTGACGACTTTGACCTTGATAT | |
| | GCTGCCTGCTGACGCTCTTGACGATTTTGACCTTGAC | |
| | ATGCTCCCCGGGTAA | |
| Tet-On 3G | MSRLDKSKVINSALELLNGVGIEGLTTRKLAQKLGVEQ | 8 |
| | PTLYWHVKNKRALLDALPIEMLDRHHTHSCPLEGESW | |
| | QDFLRNNAKSYRCALLSHRDGAKVHLGTRPTEKQYET | |
| | LENQLAFLCQQGFSLENALYALSAVGHFTLGCVLEEQE | |
| | HQVAKEERETPTTDSMPPLLKQAIELFDRQGAEPAFLFG | |
| | LELIICGLEKQLKCESGGPTDALDDFDLDMLPADALDDF | |
| | DLDMLPADALDDFDLDMLPG | |
| Promoter | Sequence | SEQ ID NO |
| TRE3G | GAGTTTACTCCCTATCAGTGATAGAGAACGTATGAAG | 9 |
| Promoter | AGTTTACTCCCTATCAGTGATAGAGAACGTATGCAGA | |
| | CTTTACTCCCTATCAGTGATAGAGAACGTATAAGGAG | |
| | TTTACTCCCTATCAGTGATAGAGAACGTATGACCAGT | |
| | TTACTCCCTATCAGTGATAGAGAACGTATCTACAGTT | |
| | TACTCCCTATCAGTGATAGAGAACGTATATCCAGTTT | |
| | ACTCCCTATCAGTGATAGAGAACGTATAAGCTTTAGG | |
| | CGTGTACGGTGGGCGCCTATAAAAGCAGAGCTCGTTT | |
| | AGTGAACCGTCAGATCGCCTGGAGCAATTCCACAAC | |
| | ACTTTTGTCTTATACCAACTTTCCGTACCACTTCCTAC | |
| | CCTCGTAAA | |

In some embodiments, a subject is injected with a polynucleotide comprising SEQ ID NOs 1-3, SEQ ID NO 7 and/or SEQ ID NO 9. In some embodiments, the polynucleotide is a plasmid or vector genome (vg).

In some embodiments, the subject is injected with at least 1×10¹⁰, at least 1×10¹¹, at least 1×10¹², at least 1×10¹³, at least 1×10¹⁴, or at least 1×10¹⁵ vector genomes.

In some embodiments, the subject is injected with between 1×10¹⁰ to 1×10¹¹, between 1×10¹¹ to 1×10¹², between 1×10¹² to 1×10¹³, between 1×10¹³ to 1×10¹⁴, or between 1×10¹⁴ to 1×10¹⁵ vector genomes.

In some embodiments, the subject receives a dosage of at least 1 mg, at least 10 mg, at least 100 mg, at least 200 mg, at least 300 mg, 400 mg, 500 mg, 600 mg, 700 mg, 800 mg, 900 mg, 1000 mg, 1500 mg, or at least 2000 mg of doxycycline.

In some embodiments, the subject receives a dosage of between 1 mg to 10 mg, between 10 mg to 100 mg, between 100 mg to 200 mg, between 200 mg to 300 mg, between 300 mg to 400 mg, between 400 mg to 500 mg, between 500 mg to 600 mg, between 600 mg to 700 mg, between 700 mg to 800 mg, between 800 mg to 900 mg, between 900 mg to 1000 mg, between 1000 to 1500 mg, or between 1500 mg to 2000 mg of doxycycline.

In some embodiments, the subject receives a dosage of at least 1 day, at least 2 days, at least 3 days, at least 4 days, at least 5 days, at least 6 days, or 7 days a week.

In some embodiments, the subject receives a cycle alternating between days with doxycycline and days with water.

In some embodiments, the subject receives a cycle of comprising doxycycline 1 day a week and water on the remaining 6 days. In some embodiments, the subject receives a cycle comprising doxycycline 2 days a week and water on the remaining 5 days. In some embodiments, the subject receives a cycle comprising doxycycline 3 days a week and water on the remaining 4 days. In some embodiments, the subject receives a cycle comprising doxycycline 4 days a week and water on the remaining 3 days. In some embodiments, the subject receives a cycle comprising doxycycline 5 days a week and water on the remaining 2 days. In some embodiments, the subject receives a cycle comprising doxycycline 6 days a week and water on the remaining 1 day.

In some embodiments, a subject receives a cycle of comprising doxycycline and water for at least 1 week, at least 2 weeks, at least 3 weeks, at least 4 weeks, at least 8 weeks, at least 12 weeks, at least, 16 weeks, at least 20 weeks, at least 24 weeks, at least 28 weeks, at least 32 weeks, at least 36 weeks, at least 40 weeks, at least 44 weeks, at least 48 weeks or at least 52 weeks.

### Telomere Length

Telomere length is associated with cell aging. As such, a cell treated with the above-mentioned polynucleotides may have increased telomere length.

In one embodiment, telomere length may be determined for a single chromosome in a cell. In some embodiments, the average telomere length or mean telomere length is measured for a single cell. In some embodiments, the average telomere length is measured for a population of cells. A change in telomere length is an increase or decrease in telomere length, in particular an increase or decrease in the average telomere length. The change may be relative to a particular time point, i.e., telomere length of an organism at time ti as compared to telomere length at some later time. A change or difference in telomere length may also be compared as against the average or mean telomere length of a particular cell population or organismal population. In some embodiments, change in telomere length is measured against a population existing at different time periods.

Samples for measuring telomeres are made using methods well known in the art. The telomere containing samples may be obtained from any tissue of any organism, including tissues of blood, brain, bone marrow, lymph, liver spleen, breast, and other tissues, including those obtained from biopsy samples. Tissue and cells may be frozen or intact. The samples may also comprise bodily fluids, such as saliva, urine, feces, cerebrospinal fluid, semen, etc. In some embodiments, the tissue or cells are non-stem cells, i.e., somatic cells since the telomeres of stem cells generally do not decrease over time due to continued expression of telomerase activity. However, in some embodiments, telomeres may be measured for stems cells in order to assess inherited telomere characteristics of an organism.

In some embodiments, the methods and compositions of the disclosure increase the median telomere length in the subject of a cell. In some embodiments, the median telomere lengths are increased by about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100%.

In some embodiments, the median telomere lengths are increased from about 1% to about 2%, from about 2% to about 3%, from about 3% to about 4%, from about 4% to about 5%, from about 5% to about 6%, from about 6% to about 7%, from about 7% to about 8%, from about 8% to about 9%, from about 9% to about 10%, from about 10% to about 11%, from about 11% to about 12%, from about 12% to about 13%, from about 13% to about 14%, from about 14% to about 15%, from about 15% to about 16%, from about 16% to about 17%, from about 17% to about 18%, from about 18% to about 19%, from about 19% to about 20%, from about 20% to about 21%, from about 21% to about 22%, from about 22% to about 23%, from about 23% to about 24%, from about 24% to about 25%, from about 25% to about 26%, from about 26% to about 27%, from about 27% to about 28%, from about 28% to about 29%, from about 29% to about 30%, from about 30% to about 31%, from about 31% to about 32%, from about 32% to about 33%, from about 33% to about 34%, from about 34% to about 35%, from about 35% to about 36%, from about 36% to about 37%, from about 37% to about 38%, from about 38% to about 39%, from about 39% to about 40%, from about 40% to about 41%, from about 41% to about 42%, from about 42% to about 43%, from about 43% to about 44%, from about 44% to about 45%, from about 45% to about 46%, from about 46% to about 47%, from about 47% to about 48%, from about 48% to about 49%, from about 49% to about 50%, from about 50% to about 51%, from about 51% to about 52%, from about 52% to about 53%, from about 53% to about 54%, from about 54% to about 55%, from about 55% to about 56%, from about 56% to about 57%, from about 57% to about 58%, from about 58% to about 59%, from about 59% to about 60%, from about 60% to about 61%, from about 61% to about 62%, from about 62% to about 63%, from about 63% to about 64%, from about 64% to about 65%, from about 65% to about 66%, from about 66% to about 67%, from about 67% to about 68%, from about 68% to about 68%, from about 69% to about 70%, from about 70% to about 71%, from about 71% to about 72%, from about 72% to about 73%, from about 73% to about 74%, from about 74% to about 75%, from about 75% to about 76%, from about 76% to about 77%, from about 77% to about 78%, from about 78% to about 79%, from about 79% to about 80%, from about 80% to about 81%, from about 81% to about 82%, from about 82% to about 83%, from about 83% to about 84%, from about 84% to about 85%, from about 85% to about 86%, from about 86% to about 87%, from about 87% to about 88%, from about 88% to about 89%, from about 89% to about 90%, from about 90% to about 91%, from about 91% to about 92%, from about 92% to about 93%, from about 93% to about 94%, from about 94% to about 95%, from about 95% to about 96%, from about 96% to about 97%, from about 97% to about 98%, from about 98% to about 99%, or from about 99% to about 100%.

In some embodiments, the methods and compositions of the disclosure increase the 20^{th} percentile telomere length in the subject of a cell. In some embodiments, the 20^{th} percentile telomere lengths are increased by about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100%.

In some embodiments, the 20^{th} percentile telomere lengths are increased from about 1% to about 2%, from about 2% to about 3%, from about 3% to about 4%, from about 4% to about 5%, from about 5% to about 6%, from about 6% to about 7%, from about 7% to about 8%, from about 8% to about 9%, from about 9% to about 10%, from about 10% to about 11%, from about 11% to about 12%, from about 12% to about 13%, from about 13% to about 14%, from about 14% to about 15%, from about 15% to about 16%, from about 16% to about 17%, from about 17% to about 18%, from about 18% to about 19%, from about 19% to about 20%, from about 20% to about 21%, from about 21% to about 22%, from about 22% to about 23%, from about 23% to about 24%, from about 24% to about 25%, from about 25% to about 26%, from about 26% to about 27%, from about 27% to about 28%, from about 28% to about 29%, from about 29% to about 30%, from about 30% to about 31%, from about 31% to about 32%, from about 32% to about 33%, from about 33% to about 34%, from about 34% to about 35%, from about 35% to about 36%, from about 36% to about 37%, from about 37% to about 38%, from about 38% to about 39%, from about 39% to about 40%, from about 40% to about 41%, from about 41% to about 42%, from about 42% to about 43%, from about 43% to about 44%, from about 44% to about 45%, from about 45% to about 46%, from about 46% to about 47%, from about 47% to about 48%, from about 48% to about 49%, from about 49% to about 50%, from about 50% to about 51%, from about 51% to about 52%, from about 52% to about 53%, from about 53% to about 54%, from about 54% to about 55%, from about 55% to about 56%, from about 56% to about 57%, from about 57% to about 58%, from about 58% to about 59%, from about 59% to about 60%, from about 60% to about 61%, from about 61% to about 62%, from about 62% to about 63%, from about 63% to about 64%, from about 64% to about 65%, from about 65% to about 66%, from about 66% to about 67%, from about 67% to about 68%, from about 68% to about 68%, from about 69% to about 70%, from about 70% to about 71%, from about 71% to about 72%, from about 72% to about 73%, from about 73% to about 74%, from about 74% to about 75%, from about 75% to about 76%, from about 76% to about 77%, from about 77% to about 78%, from about 78% to about 79%, from about 79% to about 80%, from about 80% to about 81%, from about 81% to about 82%, from about 82% to about 83%, from about 83% to about 84%, from about 84% to about 85%, from about 85% to about 86%, from about 86% to about 87%, from about 87% to about 88%, from about 88% to about 89%, from about 89% to about 90%, from about 90% to about 91%, from about 91% to about 92%, from about 92% to about 93%, from about 93% to about 94%, from about 94% to about 95%, from about 95% to about 96%, from about 96% to about 97%, from about 97% to about 98%, from about 98% to about 99%, or from about 99% to about 100%.

In some embodiments, the methods and compositions of the disclosure decrease the percent of telomeres with lengths of less than 3 Kilo base pairs (Kbps) in the cell of a subject. In some embodiments, the percent of telomeres with lengths of less than 3 Kbps are decreased by about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100%.

In some embodiments, the percent of telomeres with lengths of less than 3 Kbps are decreased from about 1% to about 2%, from about 2% to about 3%, from about 3% to about 4%, from about 4% to about 5%, from about 5% to about 6%, from about 6% to about 7%, from about 7% to about 8%, from about 8% to about 9%, from about 9% to about 10%, from about 10% to about 11%, from about 11% to about 12%, from about 12% to about 13%, from about 13% to about 14%, from about 14% to about 15%, from about 15% to about 16%, from about 16% to about 17%, from about 17% to about 18%, from about 18% to about 19%, from about 19% to about 20%, from about 20% to about 21%, from about 21% to about 22%, from about 22% to about 23%, from about 23% to about 24%, from about 24% to about 25%, from about 25% to about 26%, from about 26% to about 27%, from about 27% to about 28%, from about 28% to about 29%, from about 29% to about 30%, from about 30% to about 31%, from about 31% to about 32%, from about 32% to about 33%, from about 33% to about 34%, from about 34% to about 35%, from about 35% to about 36%, from about 36% to about 37%, from about 37% to about 38%, from about 38% to about 39%, from about 39% to about 40%, from about 40% to about 41%, from about 41% to about 42%, from about 42% to about 43%, from about 43% to about 44%, from about 44% to about 45%, from about 45% to about 46%, from about 46% to about 47%, from about 47% to about 48%, from about 48% to about 49%, from about 49% to about 50%, from about 50% to about 51%, from about 51% to about 52%, from about 52% to about 53%, from about 53% to about 54%, from about 54% to about 55%, from about 55% to about 56%, from about 56% to about 57%, from about 57% to about 58%, from about 58% to about 59%, from about 59% to about 60%, from about 60% to about 61%, from about 61% to about 62%, from about 62% to about 63%, from about 63% to about 64%, from about 64% to about 65%, from about 65% to about 66%, from about 66% to about 67%, from about 67% to about 68%, from about 68% to about 68%, from about 69% to about 70%, from about 70% to about 71%, from about 71% to about 72%, from about 72% to about 73%, from about 73% to about 74%, from about 74% to about 75%, from about 75% to about 76%, from about 76% to about 77%, from about 77% to about 78%, from about 78% to about 79%, from about 79% to about 80%, from about 80% to about 81%, from about 81% to about 82%, from about 82% to about 83%, from about 83% to about 84%, from about 84% to about 85%, from about 85% to about 86%, from about 86% to about 87%, from about 87% to about 88%, from about 88% to about 89%, from about 89% to about 90%, from about 90% to about 91%, from about 91% to about 92%, from about 92% to about 93%, from about 93% to about 94%, from about 94% to about 95%, from about 95% to about 96%, from about 96% to about 97%, from about 97% to about 98%, from about 98% to about 99%, or from about 99% to about 100%.

### DNA Methylation

DNA methylation patterns have been found to change with increasing age and contribute to age-related diseases. Methylation in many promoter regions is often accompanied by loss of gene silencing and methylation or loss of proteins that can bind to certain methylated cytosine DNA nucleotides.

One particular type of epigenetic control is cytosine-5 methylation within cytosine-phosphate-guanine (CpG) dinucleotides. Age-related DNA hypomethylation has long been observed in a variety of species, including salmon, rat and mouse. Recent studies have shown that many CpG's undergo age-related hypermethylation or hypomethylation. Previous studies have shown that age-related hypermethylation occurs on CpG islands, on bivalent chromatin domain promoters associated with key developmental genes and on polycomb family proteins. Epigenetic landscape varies significantly between tissue types, and many age-related changes depend on tissue type. Several studies have shown that age dependent CpG signatures can be defined independent of gender, tissue type, disease status and array platform.

In some embodiments, the present disclosure a multi-tissue age predictor is provided for estimating age using a set of CpG methylation markers. One advantage of the multi-organization age predictor is its wide applicability: for most tissues, it does not require any adjustment or compensation. The disclosure allows to compare the age of different parts of the human body. Furthermore, multiple tissue age predictors and CpG methylation markers allow the use of readily accessible tissues (e.g., blood, saliva, buccal cells, epidermis) to measure the age of less accessible tissues (e.g., brain, kidney, liver).

In some embodiments, a method for determining the age of a biological sample is provided, the method comprising selectively measuring the methylation level of a set of methylation markers in genomic DNA of the biological sample, the set of methylation markers comprising markers in the genes listed in U.S. Ser. No. 17/022,345, U.S. Ser. No. 17/170,487, U.S. Pat. No. 11,072,817 B2, or U.S. Pat. No. 10,435,743 B2, which are incorporated by reference.

In some embodiments, the methods and compositions of the disclosure increase the methylation level of a set of methylation markers in the cell of a subject. In some embodiments, the methylation level of a set of methylation markers are increased by about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100%.

In some embodiments, the methylation level of a set of methylation markers are increased from about 1% to about 2%, from about 2% to about 3%, from about 3% to about 4%, from about 4% to about 5%, from about 5% to about 6%, from about 6% to about 7%, from about 7% to about 8%, from about 8% to about 9%, from about 9% to about 10%, from about 10% to about 11%, from about 11% to about 12%, from about 12% to about 13%, from about 13% to about 14%, from about 14% to about 15%, from about 15% to about 16%, from about 16% to about 17%, from about 17% to about 18%, from about 18% to about 19%, from about 19% to about 20%, from about 20% to about 21%, from about 21% to about 22%, from about 22% to about 23%, from about 23% to about 24%, from about 24% to about 25%, from about 25% to about 26%, from about 26% to about 27%, from about 27% to about 28%, from about 28% to about 29%, from about 29% to about 30%, from about 30% to about 31%, from about 31% to about 32%, from about 32% to about 33%, from about 33% to about 34%, from about 34% to about 35%, from about 35% to about 36%, from about 36% to about 37%, from about 37% to about 38%, from about 38% to about 39%, from about 39% to about 40%, from about 40% to about 41%, from about 41% to about 42%, from about 42% to about 43%, from about 43% to about 44%, from about 44% to about 45%, from about 45% to about 46%, from about 46% to about 47%, from about 47% to about 48%, from about 48% to about 49%, from about 49% to about 50%, from about 50% to about 51%, from about 51% to about 52%, from about 52% to about 53%, from about 53% to about 54%, from about 54% to about 55%, from about 55% to about 56%, from about 56% to about 57%, from about 57% to about 58%, from about 58% to about 59%, from about 59% to about 60%, from about 60% to about 61%, from about 61% to about 62%, from about 62% to about 63%, from about 63% to about 64%, from about 64% to about 65%, from about 65% to about 66%, from about 66% to about 67%, from about 67% to about 68%, from about 68% to about 68%, from about 69% to about 70%, from about 70% to about 71%, from about 71% to about 72%, from about 72% to about 73%, from about 73% to about 74%, from about 74% to about 75%, from about 75% to about 76%, from about 76% to about 77%, from about 77% to about 78%, from about 78% to about 79%, from about 79% to about 80%, from about 80% to about 81%, from about 81% to about 82%, from about 82% to about 83%, from about 83% to about 84%, from about 84% to about 85%, from about 85% to about 86%, from about 86% to about 87%, from about 87% to about 88%, from about 88% to about 89%, from about 89% to about 90%, from about 90% to about 91%, from about 91% to about 92%, from about 92% to about 93%, from about 93% to about 94%, from about 94% to about 95%, from about 95% to about 96%, from about 96% to about 97%, from about 97% to about 98%, from about 98% to about 99%, or from about 99% to about 100%.

In some embodiments, the methods and compositions of the disclosure decrease the methylation level of a set of methylation markers in the cell of a subject. In some embodiments, the methylation level of a set of methylation markers are decreased by about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100%.

In some embodiments, the methylation level of a set of methylation markers are decreased from about 1% to about 2%, from about 2% to about 3%, from about 3% to about 4%, from about 4% to about 5%, from about 5% to about 6%, from about 6% to about 7%, from about 7% to about 8%, from about 8% to about 9%, from about 9% to about 10%, from about 10% to about 11%, from about 11% to about 12%, from about 12% to about 13%, from about 13% to about 14%, from about 14% to about 15%, from about 15% to about 16%, from about 16% to about 17%, from about 17% to about 18%, from about 18% to about 19%, from about 19% to about 20%, from about 20% to about 21%, from about 21% to about 22%, from about 22% to about 23%, from about 23% to about 24%, from about 24% to about 25%, from about 25% to about 26%, from about 26% to about 27%, from about 27% to about 28%, from about 28% to about 29%, from about 29% to about 30%, from about 30% to about 31%, from about 31% to about 32%, from about 32% to about 33%, from about 33% to about 34%, from about 34% to about 35%, from about 35% to about 36%, from about 36% to about 37%, from about 37% to about 38%, from about 38% to about 39%, from about 39% to about 40%, from about 40% to about 41%, from about 41% to about 42%, from about 42% to about 43%, from about 43% to about 44%, from about 44% to about 45%, from about 45% to about 46%, from about 46% to about 47%, from about 47% to about 48%, from about 48% to about 49%, from about 49% to about 50%, from about 50% to about 51%, from about 51% to about 52%, from about 52% to about 53%, from about 53% to about 54%, from about 54% to about 55%, from about 55% to about 56%, from about 56% to about 57%, from about 57% to about 58%, from about 58% to about 59%, from about 59% to about 60%, from about 60% to about 61%, from about 61% to about 62%, from about 62% to about 63%, from about 63% to about 64%, from about 64% to about 65%, from about 65% to about 66%, from about 66% to about 67%, from about 67% to about 68%, from about 68% to about 68%, from about 69% to about 70%, from about 70% to about 71%, from about 71% to about 72%, from about 72% to about 73%, from about 73% to about 74%, from about 74% to about 75%, from about 75% to about 76%, from about 76% to about 77%, from about 77% to about 78%, from about 78% to about 79%, from about 79% to about 80%, from about 80% to about 81%, from about 81% to about 82%, from about 82% to about 83%, from about 83% to about 84%, from about 84% to about 85%, from about 85% to about 86%, from about 86% to about 87%, from about 87% to about 88%, from about 88% to about 89%, from about 89% to about 90%, from about 90% to about 91%, from about 91% to about 92%, from about 92% to about 93%, from about 93% to about 94%, from about 94% to about 95%, from about 95% to about 96%, from about 96% to about 97%, from about 97% to about 98%, from about 98% to about 99%, or from about 99% to about 100%.

### Polyethylenimine (PEI)

Viral delivery systems have been traditionally used to deliver polynucleotides to cells. Several limitations exist with viral delivery systems, such as the size of polynucleotide and the tissues targeted. PEI is able to solve these issues by incorporating larger polynucleotides and targeting larger tissue types compared to viral delivery systems.

PEIs are polymeric molecules composed of repeating units of amine groups and two aliphatic carbons; a branched PEI, BPEI, may have all types of primary, secondary and tertiary amino groups while a linear PEI, LPEI, contains only secondary and primary amino groups.

PEI as a non-viral delivery system that rests precisely upon the iminoethylene monomers, which define the high density of cationic charges. The protonated amines in the polymer can form electrostatic bonds with the anionic charges present in nucleic acids, mainly due to the phosphate back bone present in both DNA and RNA. The PEI tightly compacts the nucleic acid and may allow for increased delivery to the nucleus compared to other methods.

One important characteristic of PEI polyplex formulations is the polynucleotide to PEI ratio (N:P ratio), which gives the ratio of the nitrogen groups in the PEI to the number of phosphate groups in the polynucleotide.

In some embodiments, the N:P ratio is at least 1:1, is at least 1:2, is at least 1:3, is at least 1:4, is at least 1:4, is at least 1:5, is at least 1:6, is at least 1:7, is at least 1:8, is at least 1:9, is at least 1:10, is at least 1:20, is at least 1:30, is at least 1:40, or is at least 1:50.

In some embodiments, the N:P ratio is from about 1:1 to 1:2, is from about 1:2 to 1:3, is from about 1:3 to 1:4, is from about 1:3 to 1:4, is from about 1:4 to 1:5, is from about 1:5 to 1:6, is from about 1:6 to 1:7, is from about 1:7 to 1:8, is from about 1:8 to 1:9, is from about 1:9 to 1:10, is from about 1:10 to 1:20, is from about 1:20 to 1:30, is from about 1:30 to 1:40, or is from about 1:40 to 1:50.

In some embodiments, the P:N ratio is at least 1:1, is at least 1:2, is at least 1:3, is at least 1:4, is at least 1:4, is at least 1:5, is at least 1:6, is at least 1:7, is at least 1:8, is at least 1:9, is at least 1:10, is at least 1:20, is at least 1:30, is at least 1:40, or is at least 1:50.

In some embodiments, the P:N ratio is from about 1:1 to 1:2, is from about 1:2 to 1:3, is from about 1:3 to 1:4, is from about 1:3 to 1:4, is from about 1:4 to 1:5, is from about 1:5 to 1:6, is from about 1:6 to 1:7, is from about 1:7 to 1:8, is from about 1:8 to 1:9, is from about 1:9 to 1:10, is from about 1:10 to 1:20, is from about 1:20 to 1:30, is from about 1:30 to 1:40, or is from about 1:40 to 1:50.

In some embodiments, a polynucleotide containing solution is added to a polymer containing solution, wherein the volume of the polynucleotide containing solution is equal to or exceeds the volume of the polymer containing solution. In some embodiments, the volume ratio of the a polynucleotide containing solution to the polymer containing solution is between about 1: 1 and 99:1. In some embodiments, a polynucleotide containing solution is added to a polymer containing solution, where the polynucleotide containing solution has a concentration of polynucleotide which is less than 2-fold of the concentration in the final composition. In some embodiments, the polynucleotide concentration in a mixture of the polynucleotide containing solution added to a polymer containing solution is 0.5 mg/ml or lower, 0.4 mg/ml or lower, 0.3 mg/ml or lower, 0.2 mg/ml or lower, or 0.1 mg/ml or lower. In some embodiments, the polynucleotide concentration in a polynucleotide containing solution to be added to a polymer containing solution is 1 mg/ml or lower, 0.9 mg/ml or lower, 0.8 mg/ml or lower, 0.7 mg/ml or lower, 0.6 mg/ml or lower, 0.5 mg/ml or lower, 0.4 mg/ml or lower, 0.3 mg/ml or lower, 0.2 mg/ml or lower, or 0.1 mg/ml or lower.

### Inducible Promoters

Examples of small molecule inducible promoters include, e.g. doxycycline or cumate inducible promoters. Examples of cell-autonomous promoters include, e.g., cell type-specific promoters, such as DCX. Examples of cell non-autonomous promoter include, e.g., heat induced and light induced promoters.

### Tetracycline-Inducible System

Tet technology comprises two complementary control circuits, initially described as the tTA dependent (Gossen et al. Proc Natl Acad Sci USA. 1992 Jun. 15; 89(12):5547-51) and rtTA dependent (Gossen et al. Science. 1995 Jun. 23; 268(5218): 1766-9) expression systems. They are now commonly referred to as the Tet-Off system (tTA dependent) and the Tet-On system (rtTA dependent). In each system, a recombinant tetracycline-controlled transcription factor (tTA or rtTA) interacts with a tTA/rtTA responsive promoter, Ptet, to drive expression of the gene of interest. Expression is regulated by the effector substance tetracycline (Tc) or one of its derivatives. Tet-On systems respond to doxycycline (Dox).

In some embodiments, tetracycline or doxycycline is administered to a subject to induce expression of the polynucleotide described herein.

In some embodiments, about 0.001 mg/kg, about 0.01 mg/kg, about 0.1 mg/kg, about 1 mg/kg, about 10 mg/kg, about 100 mg/kg, or about 1,000 mg/kg tetracycline or doxycycline is administered to a subject to induce expression of the polynucleotide described herein.

In some embodiments, form about 0.001 mg/kg to about 0.01 mg/kg, from about 0.01 mg/kg to about 0.1 mg/kg, from about 0.1 mg/kg to about 1 mg/kg, from about 1 mg/kg to about 10 mg/kg, from about 10 mg/kg to about 100 mg/kg, or from about 100 mg/kg to about 1,000 mg/kg tetracycline or doxycycline is administered to a subject to induce expression of the polynucleotide described herein.

### Lifespan

In some embodiments, the methods and compositions increase the lifespan of a subject compared to a subject that was not administered the methods of compositions of the current disclosure.

In some embodiments, the lifespan is increased by about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% in a subject administered the methods and compositions of the current disclosure compared to a subject not administered the methods and compositions.

### Blood Flow

In some embodiments, the methods and compositions increase the blood flow to the skin (ruddiness) of a subject compared to baseline.

In some embodiments, the blood flow to the skin is increased by about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% in a subject administered the methods and compositions of the current disclosure compared to baseline.

### Alopecia Indicators

Indicators of alopecia include, but are not limited to, a decrease in anagen and catagen hair follicles, increased adnexal atrophy and increased mononuclear perivascular inflammatory infiltrate.

### Hair Follicle Stage

Hair follicles (HFs) undergo life-long cyclical transformations, progressing through stages of rapid growth (anagen), regression (catagen), and relative “quiescence” (telogen). Since HF cycling abnormalities underlie many human hair growth disorders, the accurate classification of individual cycle stages within skin biopsies is clinically important and essential for hair research.

In normal hair cycling, ˜90% of follicles are in anagen phase, 1% in catagen phase and 9% in telogen phase at any given time, with the proportion of follicles in anagen phase declining with age. See Witing D A, Int J Dermatol. 1998; 37(8):561-566 and Courtois et al., Br J Dermatol. 1995; 132(1):86-93.

In some embodiments, the methods and compositions of the disclosure increase the number of anagen phase and catagen phase hair follicles. In some embodiments, the methods and compositions of the disclosure increase the percentage of anagen phase follicles in a subject compared to a subject that is not administered the composition or methods of the disclosure. In some embodiments, the number of anagen phase hair follicles are increased by about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100%.

In some embodiments, the number of anagen phase hair follicles are increased from about 1% to about 2%, from about 2% to about 3%, from about 3% to about 4%, from about 4% to about 5%, from about 5% to about 6%, from about 6% to about 7%, from about 7% to about 8%, from about 8% to about 9%, from about 9% to about 10%, from about 10% to about 11%, from about 11% to about 12%, from about 12% to about 13%, from about 13% to about 14%, from about 14% to about 15%, from about 15% to about 16%, from about 16% to about 17%, from about 17% to about 18%, from about 18% to about 19%, from about 19% to about 20%, from about 20% to about 21%, from about 21% to about 22%, from about 22% to about 23%, from about 23% to about 24%, from about 24% to about 25%, from about 25% to about 26%, from about 26% to about 27%, from about 27% to about 28%, from about 28% to about 29%, from about 29% to about 30%, from about 30% to about 31%, from about 31% to about 32%, from about 32% to about 33%, from about 33% to about 34%, from about 34% to about 35%, from about 35% to about 36%, from about 36% to about 37%, from about 37% to about 38%, from about 38% to about 39%, from about 39% to about 40%, from about 40% to about 41%, from about 41% to about 42%, from about 42% to about 43%, from about 43% to about 44%, from about 44% to about 45%, from about 45% to about 46%, from about 46% to about 47%, from about 47% to about 48%, from about 48% to about 49%, from about 49% to about 50%, from about 50% to about 51%, from about 51% to about 52%, from about 52% to about 53%, from about 53% to about 54%, from about 54% to about 55%, from about 55% to about 56%, from about 56% to about 57%, from about 57% to about 58%, from about 58% to about 59%, from about 59% to about 60%, from about 60% to about 61%, from about 61% to about 62%, from about 62% to about 63%, from about 63% to about 64%, from about 64% to about 65%, from about 65% to about 66%, from about 66% to about 67%, from about 67% to about 68%, from about 68% to about 68%, from about 69% to about 70%, from about 70% to about 71%, from about 71% to about 72%, from about 72% to about 73%, from about 73% to about 74%, from about 74% to about 75%, from about 75% to about 76%, from about 76% to about 77%, from about 77% to about 78%, from about 78% to about 79%, from about 79% to about 80%, from about 80% to about 81%, from about 81% to about 82%, from about 82% to about 83%, from about 83% to about 84%, from about 84% to about 85%, from about 85% to about 86%, from about 86% to about 87%, from about 87% to about 88%, from about 88% to about 89%, from about 89% to about 90%, from about 90% to about 91%, from about 91% to about 92%, from about 92% to about 93%, from about 93% to about 94%, from about 94% to about 95%, from about 95% to about 96%, from about 96% to about 97%, from about 97% to about 98%, from about 98% to about 99%, or from about 99% to about 100%.

In some embodiments, the number of catagen phase hair follicles are increased by about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100%.

In some embodiments, the number of catagen phase hair follicles are increased from about 1% to about 2%, from about 2% to about 3%, from about 3% to about 4%, from about 4% to about 5%, from about 5% to about 6%, from about 6% to about 7%, from about 7% to about 8%, from about 8% to about 9%, from about 9% to about 10%, from about 10% to about 11%, from about 11% to about 12%, from about 12% to about 13%, from about 13% to about 14%, from about 14% to about 15%, from about 15% to about 16%, from about 16% to about 17%, from about 17% to about 18%, from about 18% to about 19%, from about 19% to about 20%, from about 20% to about 21%, from about 21% to about 22%, from about 22% to about 23%, from about 23% to about 24%, from about 24% to about 25%, from about 25% to about 26%, from about 26% to about 27%, from about 27% to about 28%, from about 28% to about 29%, from about 29% to about 30%, from about 30% to about 31%, from about 31% to about 32%, from about 32% to about 33%, from about 33% to about 34%, from about 34% to about 35%, from about 35% to about 36%, from about 36% to about 37%, from about 37% to about 38%, from about 38% to about 39%, from about 39% to about 40%, from about 40% to about 41%, from about 41% to about 42%, from about 42% to about 43%, from about 43% to about 44%, from about 44% to about 45%, from about 45% to about 46%, from about 46% to about 47%, from about 47% to about 48%, from about 48% to about 49%, from about 49% to about 50%, from about 50% to about 51%, from about 51% to about 52%, from about 52% to about 53%, from about 53% to about 54%, from about 54% to about 55%, from about 55% to about 56%, from about 56% to about 57%, from about 57% to about 58%, from about 58% to about 59%, from about 59% to about 60%, from about 60% to about 61%, from about 61% to about 62%, from about 62% to about 63%, from about 63% to about 64%, from about 64% to about 65%, from about 65% to about 66%, from about 66% to about 67%, from about 67% to about 68%, from about 68% to about 68%, from about 69% to about 70%, from about 70% to about 71%, from about 71% to about 72%, from about 72% to about 73%, from about 73% to about 74%, from about 74% to about 75%, from about 75% to about 76%, from about 76% to about 77%, from about 77% to about 78%, from about 78% to about 79%, from about 79% to about 80%, from about 80% to about 81%, from about 81% to about 82%, from about 82% to about 83%, from about 83% to about 84%, from about 84% to about 85%, from about 85% to about 86%, from about 86% to about 87%, from about 87% to about 88%, from about 88% to about 89%, from about 89% to about 90%, from about 90% to about 91%, from about 91% to about 92%, from about 92% to about 93%, from about 93% to about 94%, from about 94% to about 95%, from about 95% to about 96%, from about 96% to about 97%, from about 97% to about 98%, from about 98% to about 99%, or from about 99% to about 100%.

### Adnexal Atrophy

Adnexal atrophy is characterized by smaller and reduced numbers of hair follicles, and sebaceous gland atrophy is characterized by smaller and reduced numbers of sebaceous glands. Adnexal atrophy may correspond to the gross observation of alopecia.

In some embodiments, the methods and compositions of the decreases adnexal atrophy. In some embodiments, the methods and compositions of the disclosure increase the percentage of adnexal atrophy observed in a subject compared to a subject that is not administered the composition or methods of the disclosure. In some embodiments, the observation of adnexal atrophy are decreased by about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100%.

In some embodiments, the observation of adnexal atrophy are decreased from about 1% to about 2%, from about 2% to about 3%, from about 3% to about 4%, from about 4% to about 5%, from about 5% to about 6%, from about 6% to about 7%, from about 7% to about 8%, from about 8% to about 9%, from about 9% to about 10%, from about 10% to about 11%, from about 11% to about 12%, from about 12% to about 13%, from about 13% to about 14%, from about 14% to about 15%, from about 15% to about 16%, from about 16% to about 17%, from about 17% to about 18%, from about 18% to about 19%, from about 19% to about 20%, from about 20% to about 21%, from about 21% to about 22%, from about 22% to about 23%, from about 23% to about 24%, from about 24% to about 25%, from about 25% to about 26%, from about 26% to about 27%, from about 27% to about 28%, from about 28% to about 29%, from about 29% to about 30%, from about 30% to about 31%, from about 31% to about 32%, from about 32% to about 33%, from about 33% to about 34%, from about 34% to about 35%, from about 35% to about 36%, from about 36% to about 37%, from about 37% to about 38%, from about 38% to about 39%, from about 39% to about 40%, from about 40% to about 41%, from about 41% to about 42%, from about 42% to about 43%, from about 43% to about 44%, from about 44% to about 45%, from about 45% to about 46%, from about 46% to about 47%, from about 47% to about 48%, from about 48% to about 49%, from about 49% to about 50%, from about 50% to about 51%, from about 51% to about 52%, from about 52% to about 53%, from about 53% to about 54%, from about 54% to about 55%, from about 55% to about 56%, from about 56% to about 57%, from about 57% to about 58%, from about 58% to about 59%, from about 59% to about 60%, from about 60% to about 61%, from about 61% to about 62%, from about 62% to about 63%, from about 63% to about 64%, from about 64% to about 65%, from about 65% to about 66%, from about 66% to about 67%, from about 67% to about 68%, from about 68% to about 68%, from about 69% to about 70%, from about 70% to about 71%, from about 71% to about 72%, from about 72% to about 73%, from about 73% to about 74%, from about 74% to about 75%, from about 75% to about 76%, from about 76% to about 77%, from about 77% to about 78%, from about 78% to about 79%, from about 79% to about 80%, from about 80% to about 81%, from about 81% to about 82%, from about 82% to about 83%, from about 83% to about 84%, from about 84% to about 85%, from about 85% to about 86%, from about 86% to about 87%, from about 87% to about 88%, from about 88% to about 89%, from about 89% to about 90%, from about 90% to about 91%, from about 91% to about 92%, from about 92% to about 93%, from about 93% to about 94%, from about 94% to about 95%, from about 95% to about 96%, from about 96% to about 97%, from about 97% to about 98%, from about 98% to about 99%, or from about 99% to about 100%.

### Mononuclear Perivascular Inflammatory Infiltrates

Alopecia is associated with inflammation in the upper dermis and damage to the hair follicle infundibulum. Inflammation in the upper dermis may be assessed by observing mononuclear perivascular inflammatory infiltrates.

In some embodiments, the methods and compositions of the disclosure decreases mononuclear perivascular inflammatory infiltrates in the upper dermis. In some embodiments, the methods and compositions of the disclosure decreases mononuclear perivascular inflammatory infiltrates in the upper dermis in a subject compared to a subject that is not administered the composition or methods of the disclosure. In some embodiments, the mononuclear perivascular inflammatory infiltrates in the upper dermis is decreased by about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100%.

In some embodiments, the mononuclear perivascular inflammatory infiltrates in the upper dermis is decreased from about 1% to about 2%, from about 2% to about 3%, from about 3% to about 4%, from about 4% to about 5%, from about 5% to about 6%, from about 6% to about 7%, from about 7% to about 8%, from about 8% to about 9%, from about 9% to about 10%, from about 10% to about 11%, from about 11% to about 12%, from about 12% to about 13%, from about 13% to about 14%, from about 14% to about 15%, from about 15% to about 16%, from about 16% to about 17%, from about 17% to about 18%, from about 18% to about 19%, from about 19% to about 20%, from about 20% to about 21%, from about 21% to about 22%, from about 22% to about 23%, from about 23% to about 24%, from about 24% to about 25%, from about 25% to about 26%, from about 26% to about 27%, from about 27% to about 28%, from about 28% to about 29%, from about 29% to about 30%, from about 30% to about 31%, from about 31% to about 32%, from about 32% to about 33%, from about 33% to about 34%, from about 34% to about 35%, from about 35% to about 36%, from about 36% to about 37%, from about 37% to about 38%, from about 38% to about 39%, from about 39% to about 40%, from about 40% to about 41%, from about 41% to about 42%, from about 42% to about 43%, from about 43% to about 44%, from about 44% to about 45%, from about 45% to about 46%, from about 46% to about 47%, from about 47% to about 48%, from about 48% to about 49%, from about 49% to about 50%, from about 50% to about 51%, from about 51% to about 52%, from about 52% to about 53%, from about 53% to about 54%, from about 54% to about 55%, from about 55% to about 56%, from about 56% to about 57%, from about 57% to about 58%, from about 58% to about 59%, from about 59% to about 60%, from about 60% to about 61%, from about 61% to about 62%, from about 62% to about 63%, from about 63% to about 64%, from about 64% to about 65%, from about 65% to about 66%, from about 66% to about 67%, from about 67% to about 68%, from about 68% to about 68%, from about 69% to about 70%, from about 70% to about 71%, from about 71% to about 72%, from about 72% to about 73%, from about 73% to about 74%, from about 74% to about 75%, from about 75% to about 76%, from about 76% to about 77%, from about 77% to about 78%, from about 78% to about 79%, from about 79% to about 80%, from about 80% to about 81%, from about 81% to about 82%, from about 82% to about 83%, from about 83% to about 84%, from about 84% to about 85%, from about 85% to about 86%, from about 86% to about 87%, from about 87% to about 88%, from about 88% to about 89%, from about 89% to about 90%, from about 90% to about 91%, from about 91% to about 92%, from about 92% to about 93%, from about 93% to about 94%, from about 94% to about 95%, from about 95% to about 96%, from about 96% to about 97%, from about 97% to about 98%, from about 98% to about 99%, or from about 99% to about 100%.

### Hair Loss

In some embodiments, the methods and compositions of the disclosure decreases hair loss. In some embodiments, the methods and compositions of the disclosure decreases hair loss in a subject compared to a subject that is not administered the composition or methods of the disclosure.

In some embodiments, the is hair loss is decreased by about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100%.

In some embodiments, the hair loss is decreased from about 1% to about 2%, from about 2% to about 3%, from about 3% to about 4%, from about 4% to about 5%, from about 5% to about 6%, from about 6% to about 7%, from about 7% to about 8%, from about 8% to about 9%, from about 9% to about 10%, from about 10% to about 11%, from about 11% to about 12%, from about 12% to about 13%, from about 13% to about 14%, from about 14% to about 15%, from about 15% to about 16%, from about 16% to about 17%, from about 17% to about 18%, from about 18% to about 19%, from about 19% to about 20%, from about 20% to about 21%, from about 21% to about 22%, from about 22% to about 23%, from about 23% to about 24%, from about 24% to about 25%, from about 25% to about 26%, from about 26% to about 27%, from about 27% to about 28%, from about 28% to about 29%, from about 29% to about 30%, from about 30% to about 31%, from about 31% to about 32%, from about 32% to about 33%, from about 33% to about 34%, from about 34% to about 35%, from about 35% to about 36%, from about 36% to about 37%, from about 37% to about 38%, from about 38% to about 39%, from about 39% to about 40%, from about 40% to about 41%, from about 41% to about 42%, from about 42% to about 43%, from about 43% to about 44%, from about 44% to about 45%, from about 45% to about 46%, from about 46% to about 47%, from about 47% to about 48%, from about 48% to about 49%, from about 49% to about 50%, from about 50% to about 51%, from about 51% to about 52%, from about 52% to about 53%, from about 53% to about 54%, from about 54% to about 55%, from about 55% to about 56%, from about 56% to about 57%, from about 57% to about 58%, from about 58% to about 59%, from about 59% to about 60%, from about 60% to about 61%, from about 61% to about 62%, from about 62% to about 63%, from about 63% to about 64%, from about 64% to about 65%, from about 65% to about 66%, from about 66% to about 67%, from about 67% to about 68%, from about 68% to about 68%, from about 69% to about 70%, from about 70% to about 71%, from about 71% to about 72%, from about 72% to about 73%, from about 73% to about 74%, from about 74% to about 75%, from about 75% to about 76%, from about 76% to about 77%, from about 77% to about 78%, from about 78% to about 79%, from about 79% to about 80%, from about 80% to about 81%, from about 81% to about 82%, from about 82% to about 83%, from about 83% to about 84%, from about 84% to about 85%, from about 85% to about 86%, from about 86% to about 87%, from about 87% to about 88%, from about 88% to about 89%, from about 89% to about 90%, from about 90% to about 91%, from about 91% to about 92%, from about 92% to about 93%, from about 93% to about 94%, from about 94% to about 95%, from about 95% to about 96%, from about 96% to about 97%, from about 97% to about 98%, from about 98% to about 99%, or from about 99% to about 100%.

### Skin Elasticity and Plasticity

In some embodiments, the methods and compositions of the disclosure increases skin elasticity and plasticity. In some embodiments, the methods and compositions of the disclosure increases skin elasticity and plasticity in a subject compared to a subject that is not administered the composition or methods of the disclosure.

In some embodiments, the skin elasticity and plasticity is increased by about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100%.

In some embodiments, the skin elasticity and plasticity is increased from about 1% to about 2%, from about 2% to about 3%, from about 3% to about 4%, from about 4% to about 5%, from about 5% to about 6%, from about 6% to about 7%, from about 7% to about 8%, from about 8% to about 9%, from about 9% to about 10%, from about 10% to about 11%, from about 11% to about 12%, from about 12% to about 13%, from about 13% to about 14%, from about 14% to about 15%, from about 15% to about 16%, from about 16% to about 17%, from about 17% to about 18%, from about 18% to about 19%, from about 19% to about 20%, from about 20% to about 21%, from about 21% to about 22%, from about 22% to about 23%, from about 23% to about 24%, from about 24% to about 25%, from about 25% to about 26%, from about 26% to about 27%, from about 27% to about 28%, from about 28% to about 29%, from about 29% to about 30%, from about 30% to about 31%, from about 31% to about 32%, from about 32% to about 33%, from about 33% to about 34%, from about 34% to about 35%, from about 35% to about 36%, from about 36% to about 37%, from about 37% to about 38%, from about 38% to about 39%, from about 39% to about 40%, from about 40% to about 41%, from about 41% to about 42%, from about 42% to about 43%, from about 43% to about 44%, from about 44% to about 45%, from about 45% to about 46%, from about 46% to about 47%, from about 47% to about 48%, from about 48% to about 49%, from about 49% to about 50%, from about 50% to about 51%, from about 51% to about 52%, from about 52% to about 53%, from about 53% to about 54%, from about 54% to about 55%, from about 55% to about 56%, from about 56% to about 57%, from about 57% to about 58%, from about 58% to about 59%, from about 59% to about 60%, from about 60% to about 61%, from about 61% to about 62%, from about 62% to about 63%, from about 63% to about 64%, from about 64% to about 65%, from about 65% to about 66%, from about 66% to about 67%, from about 67% to about 68%, from about 68% to about 68%, from about 69% to about 70%, from about 70% to about 71%, from about 71% to about 72%, from about 72% to about 73%, from about 73% to about 74%, from about 74% to about 75%, from about 75% to about 76%, from about 76% to about 77%, from about 77% to about 78%, from about 78% to about 79%, from about 79% to about 80%, from about 80% to about 81%, from about 81% to about 82%, from about 82% to about 83%, from about 83% to about 84%, from about 84% to about 85%, from about 85% to about 86%, from about 86% to about 87%, from about 87% to about 88%, from about 88% to about 89%, from about 89% to about 90%, from about 90% to about 91%, from about 91% to about 92%, from about 92% to about 93%, from about 93% to about 94%, from about 94% to about 95%, from about 95% to about 96%, from about 96% to about 97%, from about 97% to about 98%, from about 98% to about 99%, or from about 99% to about 100%.

### Fertility

In some embodiments, the methods and compositions increase the fertility of a subject compared to a subject that was not administered the methods of compositions of the current disclosure.

In some embodiments, the fertility is increased by about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% in a subject administered the methods and compositions of the current disclosure compared to a subject not administered the methods and compositions.

### EXAMPLES

### Example 1: Evaluation for Immune Response for AAV-Mediated Gene Delivery in Rodents

### Purpose

The purpose of this experiment was to determine if multiple doses of AAV could be delivered without eliciting an immune response.

### Methods

A total of male 4 Balb/c mice, 6 weeks old, were used in the study. All animals were weighed using an electronic balance, Scout Pro 202 (OHAUS Corporation, Parsippany, NJ, U.S.A.) and given a clinical examination to ensure that they were in good condition. They were housed 4 per cage. An inspection was performed to ensure their suitability for the study before injection. The animals were maintained in a HEPA-filtered environment in a Micro-VENT full ventilation rodent housing system (Allentown Caging Equipment Co., Allentown, NJ, U.S.A) at AntiCancer, Inc. Animal room controls were set to maintain temperature and relative humidity at 22° C.±2° C. and 55%±15%, respectively. The rooms were lit by artificial light for 12 hours each day. Cages and bedding were autoclaved. Water was purified by Milli-Q Biocel System (Millipore, Billerica, MA, U.S.A.), autoclaved and supplied ad libitum to each cage via water bottles. Autoclavable rodent diet 5010 was obtained from PMI Nutrition International Inc. (Brentwood, MO, U.S.A.).

AAV9-CMV-eGFP was injected to tail vein on September 16^{th} and AAV9-CMV-RFP was injected to tail vein on December 16^{th} as summarized in Table 1.

| | | Injection | Injection |
|---|---|---|---|
| | | volume | volume |
| Group | n | (GFP) | (RFP) |
| 1 | 1 | 100 ul | 100 ul |
| 2 | 1 | 200 ul | 200 ul |
| 3 | 1 | 300 ul | 300 ul |
| 4 | 1 | 400 ul | 400 ul |

Whole organ images were made and the mean densities of organs were checked using UVP ChemStudio (Analytikjena Co. Germany). Femur shaft as bone, right colon without feces as colon, ear as skin, upper leg muscle as muscle were used to check mean densities. Liver, brain, spleen and heart were used in intact forms to check mean densities. Both sides of kidneys and lungs were analyzed and higher intensities were recorded.

On January 17^{th}, all mice were sacrificed and 10 organs (heart, brain, lung, liver, spleen, kidney colon, skin, muscle and bone) were harvested. Using liquid nitrogen and Tissue-Tek O.C.T compound (Sakura Finetek USA, Inc. Torrance, CA, U.S.A.), the whole organs were made as frozen blocks and have been keeping on −80° C.

The body weight was measured at the days of injection and the day of end study and are summarized in Table 2.

TABLE 2
Mouse Body Weights

| Group | Sep. 16^{th} | Dec. 16^{th} | Jan. 17^{th} |
|---|---|---|---|
| 1 | 18.9 g | 27.0 g | 29.3 g |
| 2 | 24.4 g | 32.0 g | 33.5 g |
| 3 | 23.5 g | 28.8 g | 30.1 g |
| 4 | 22.2 g | 30.0 g | 30.2 g |

The average of intensities of all pixels of the region, minus average intensity of background pixels, is shown in Table 3.

TABLE 3
Mean density of whole organs

| | Group 1 | | Group 2 | | Group 3 | | Group 4 | |
|---|---|---|---|---|---|---|---|---|
| Fluorescence | 1 GFP | 1 RFP | 2 GFP | 2 RFP | 3 GFP | 3 RFP | 4 GFP | 4 RFP |
| Bone | 786.71 | 220.79 | 1016.74 | 308.06 | 1686.75 | 152.03 | 1347.23 | 125.77 |
| Brain | 190.57 | 62.71 | 206.85 | 90.27 | 196.63 | 67.01 | 82.07 | 32.96 |
| Colon | 116.73 | 33.87 | 205.49 | 40.67 | 86.83 | 19.95 | 206.89 | 123.38 |
| Heart | 260.78 | 12.92 | 504.33 | 24.79 | 331.02 | 7.61 | 1757.91 | 26.96 |
| Kidney | 129.09 | 22.93 | 211.06 | 50.91 | 189.19 | 50.19 | 43.64 | 30.45 |
| Liver | 156.86 | 25.73 | 236.70 | 21.52 | 58.72 | 24.59 | 160.30 | 17.01 |
| Lung | 54.06 | 43.85 | 45.78 | 31.88 | 47.74 | 39.16 | 9.56 | 29.19 |
| Muscle | 703.33 | 41.18 | 602.77 | 32.14 | 772.50 | 35.51 | 2499.63 | 57.09 |
| Skin | 481.17 | 75.83 | 491.25 | 76.29 | 370.96 | 54.99 | 395.33 | 72.38 |
| Spleen | −6.53 | −2.40 | 0.14 | −0.21 | −1.34 | 1.91 | 8.49 | 0.32 |
| Sum | 2872.77 | 537.41 | 3521.11 | 676.32 | 3739 | 452.95 | 6511.05 | 515.51 |

### Results

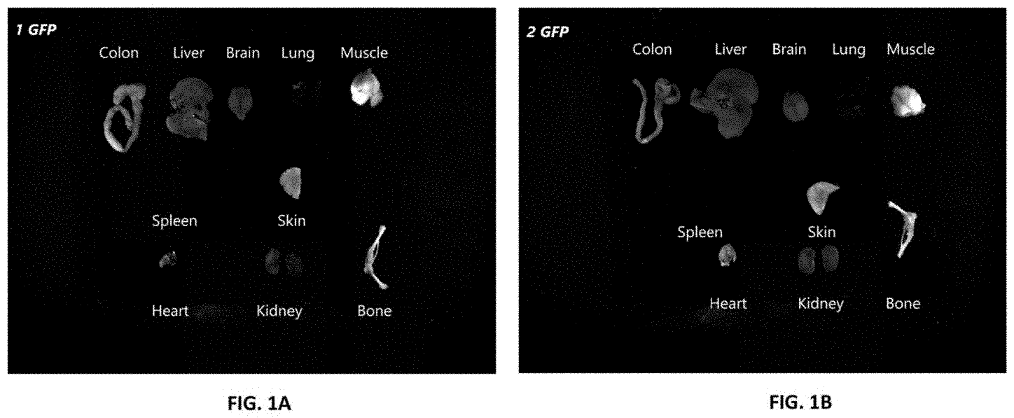

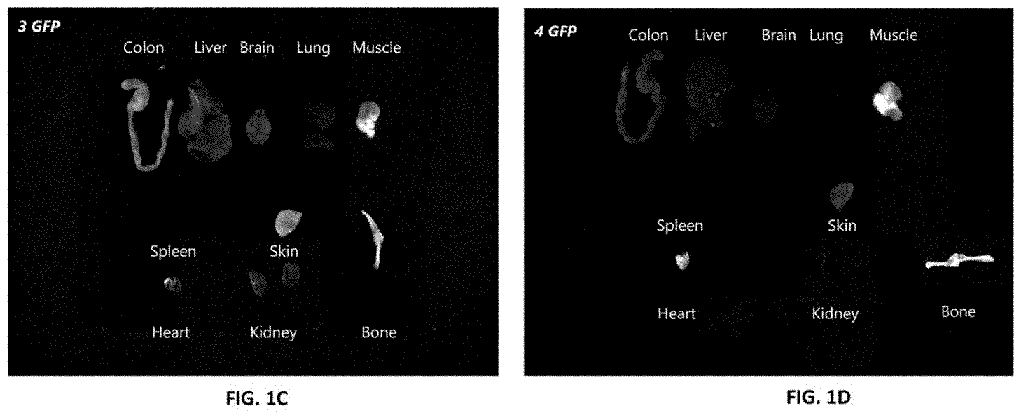

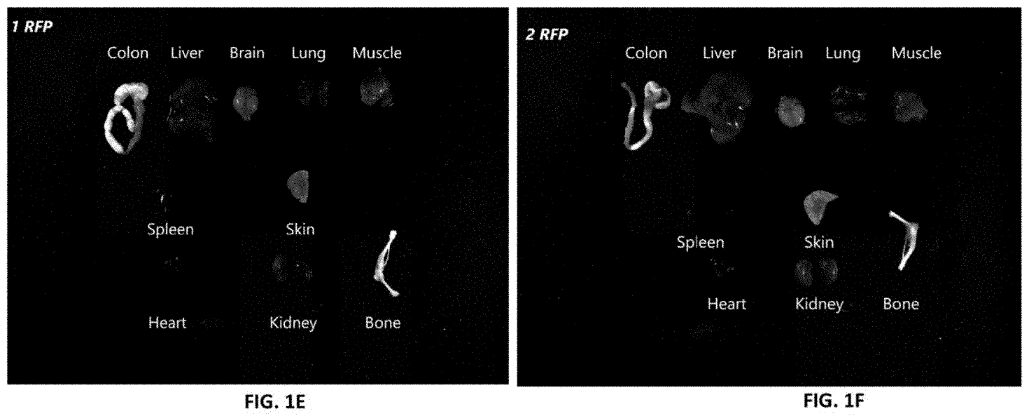

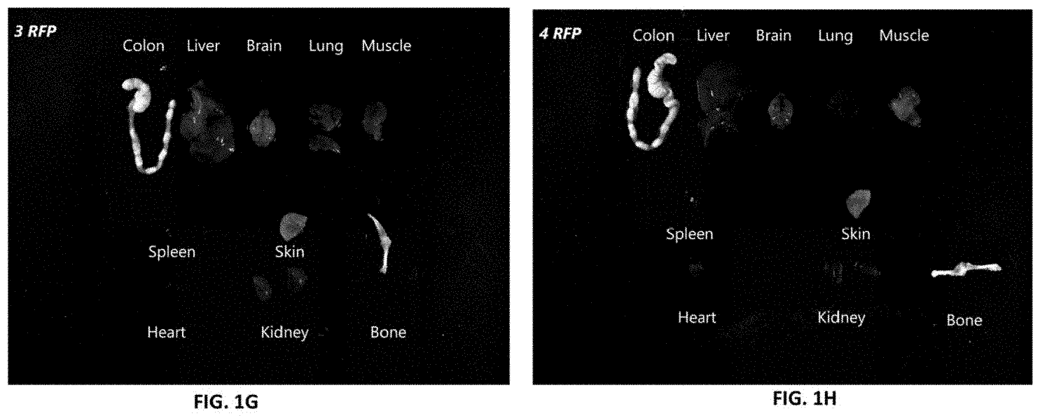

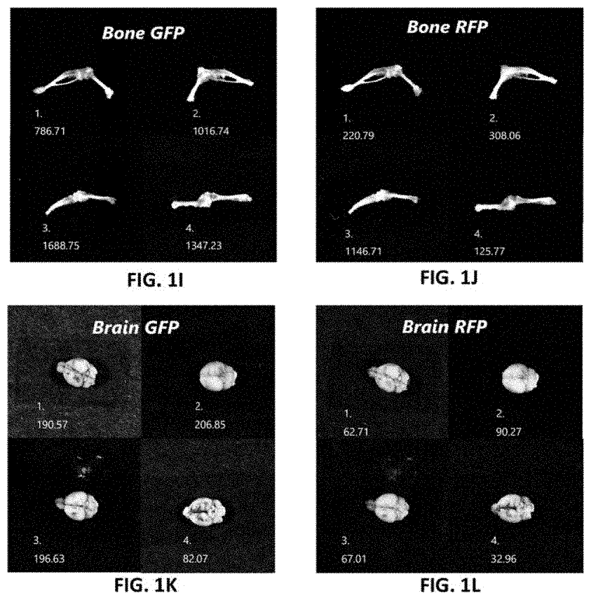

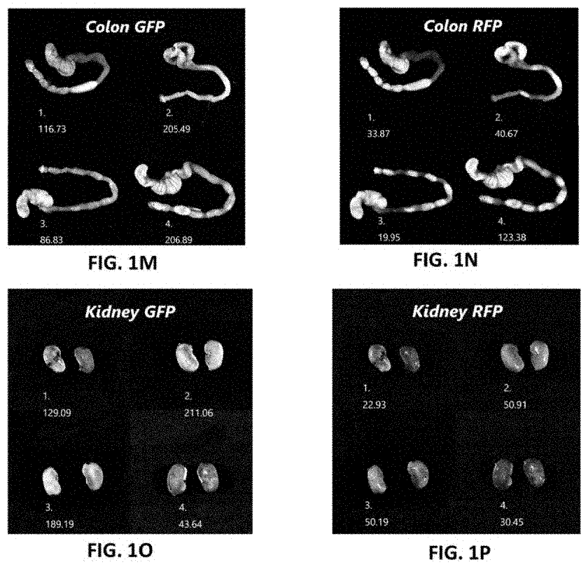

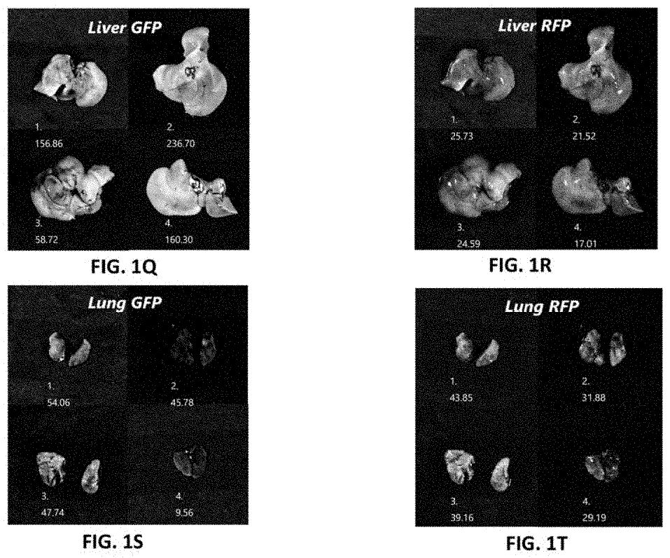

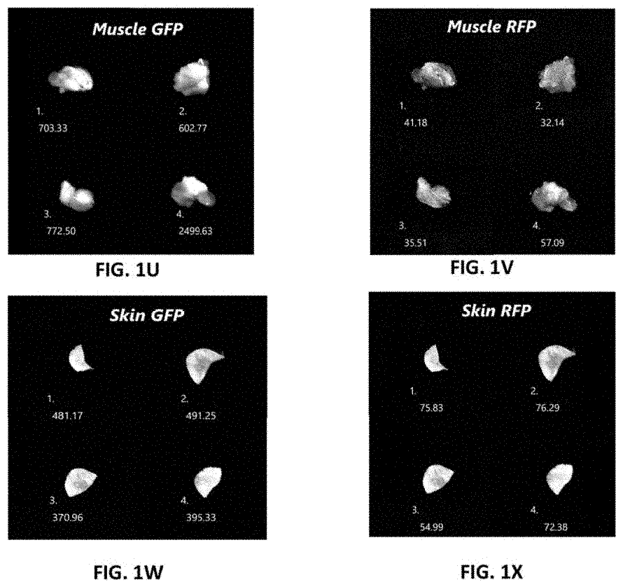

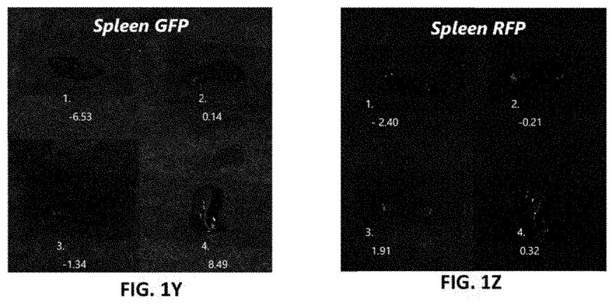

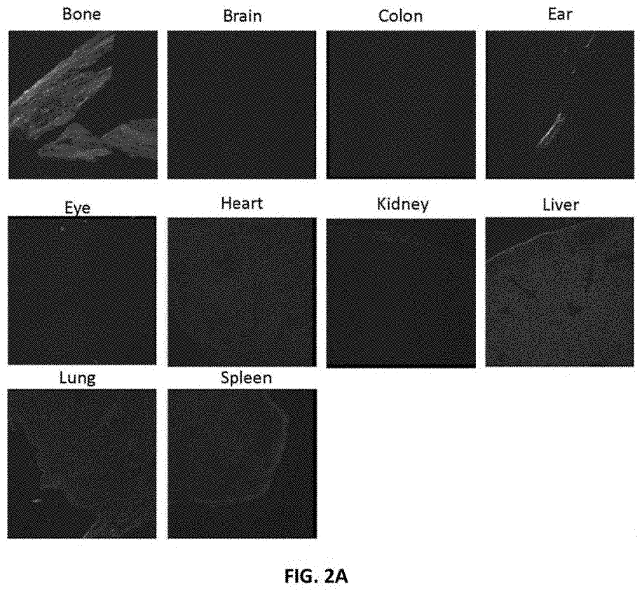

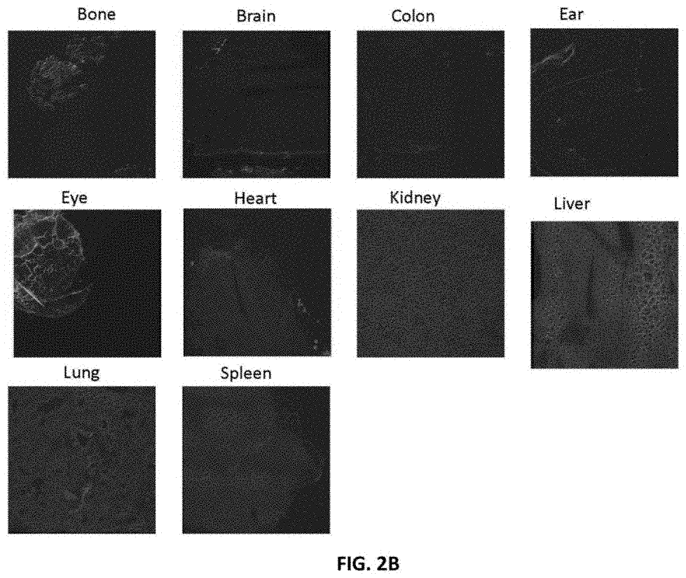

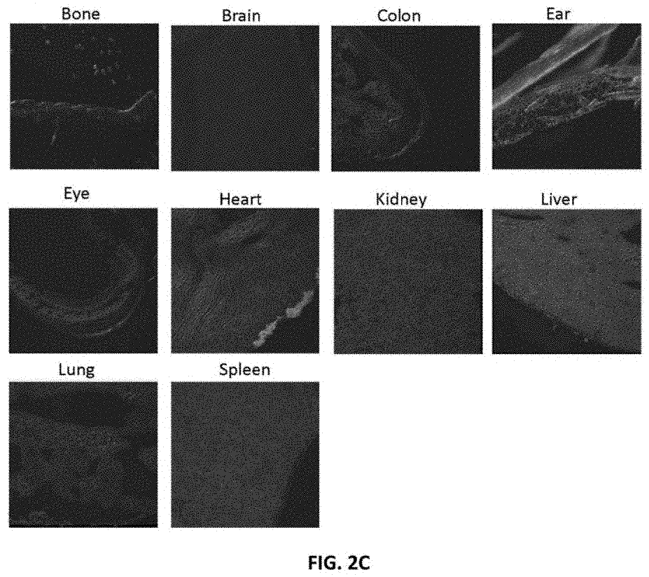

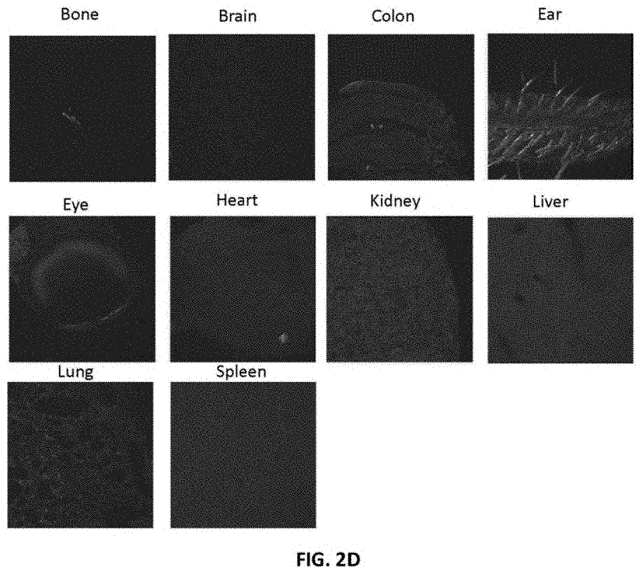

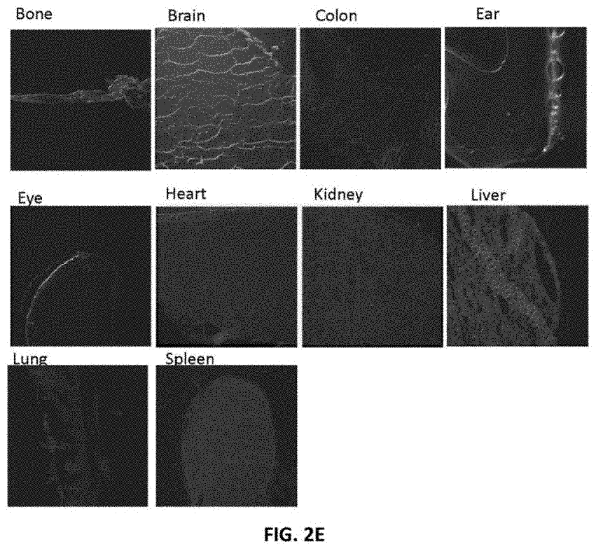

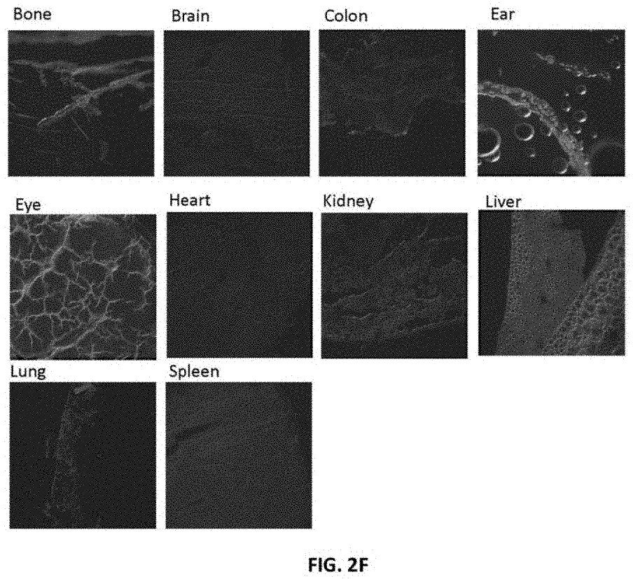

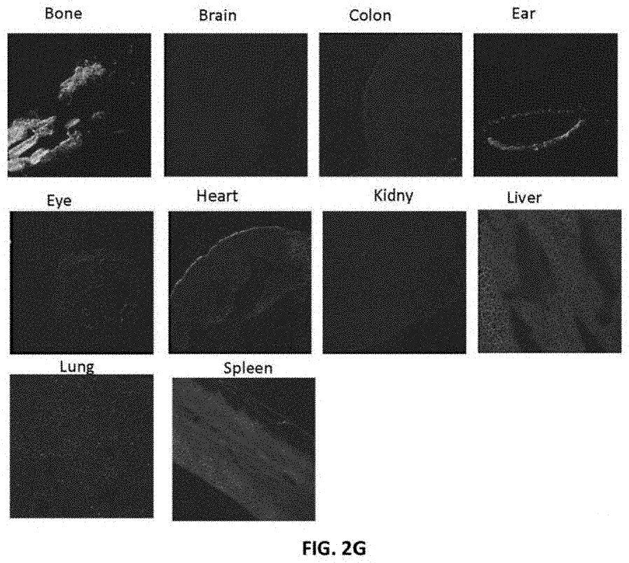

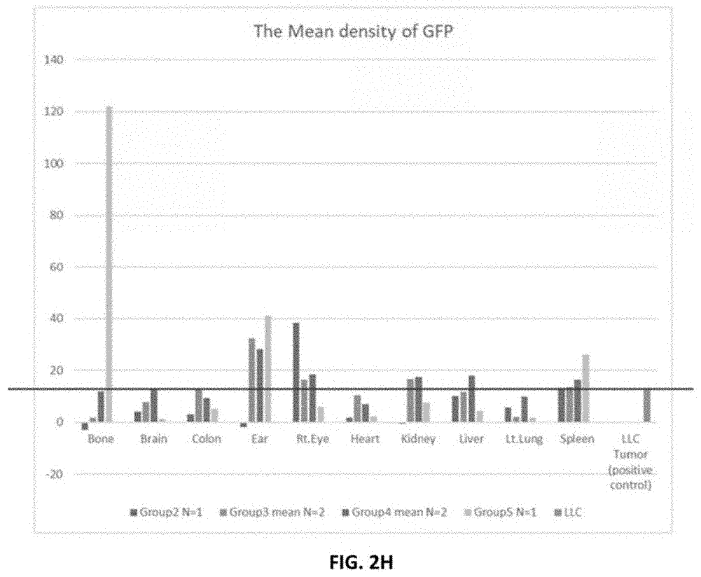

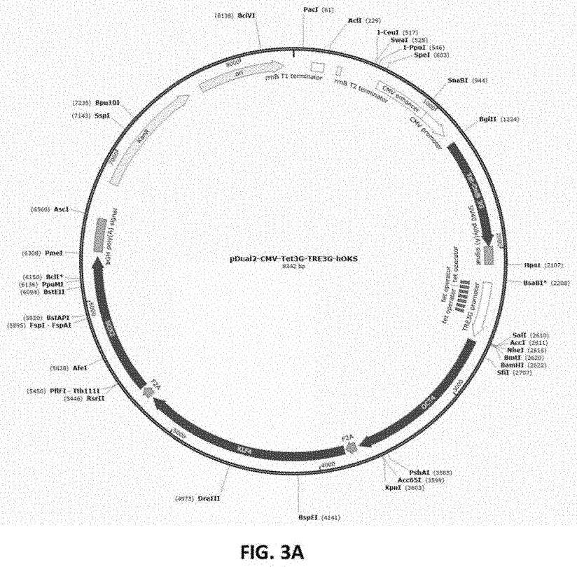

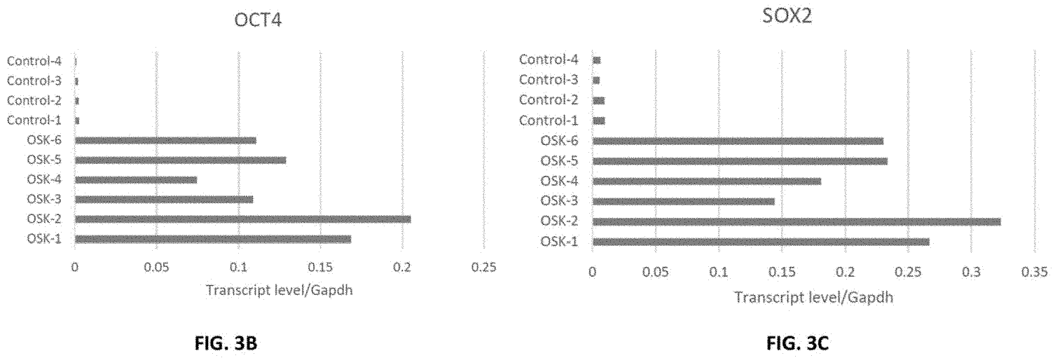

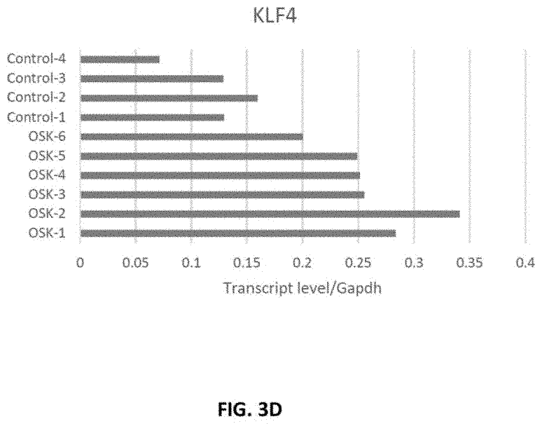

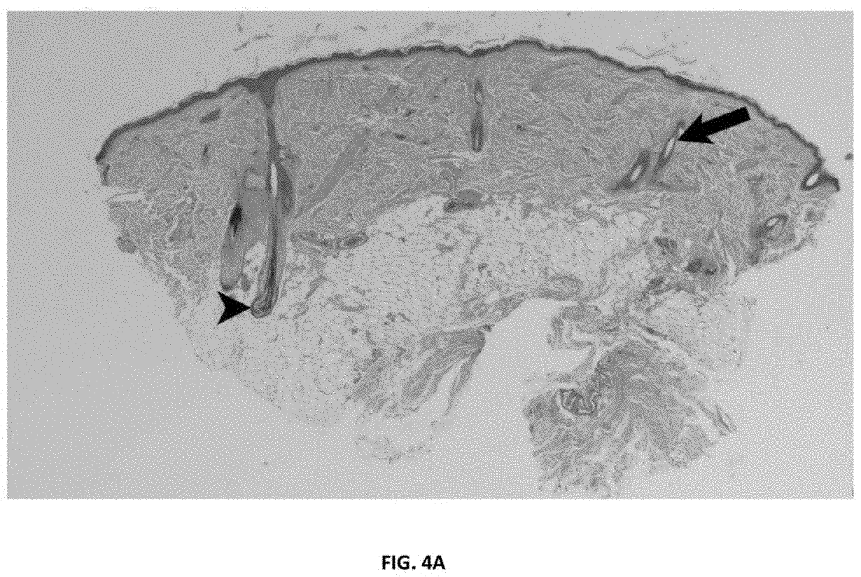

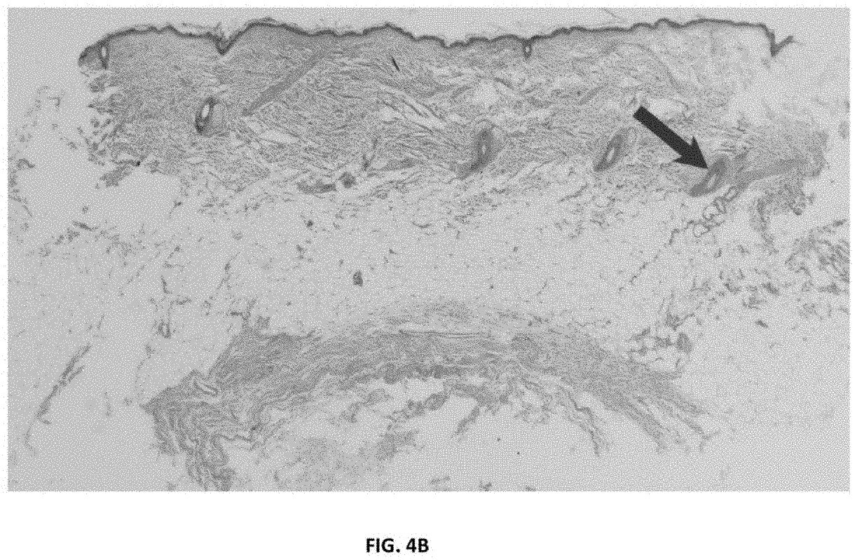

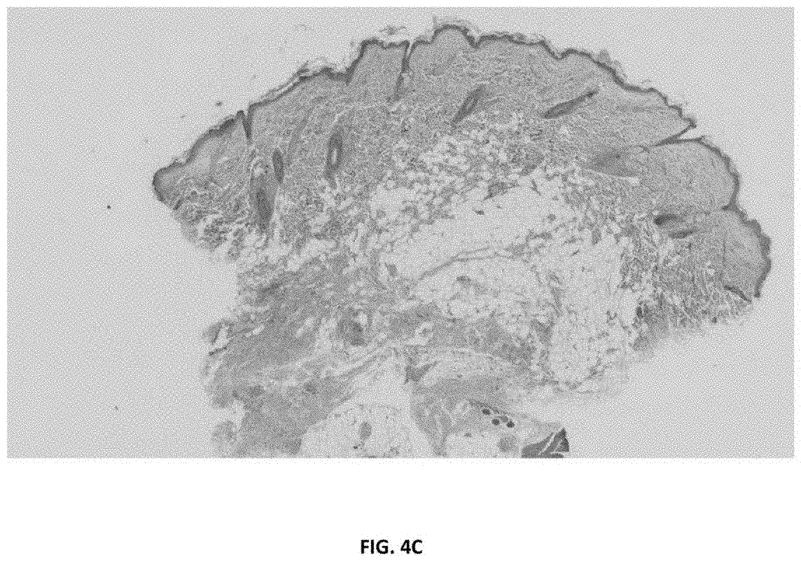

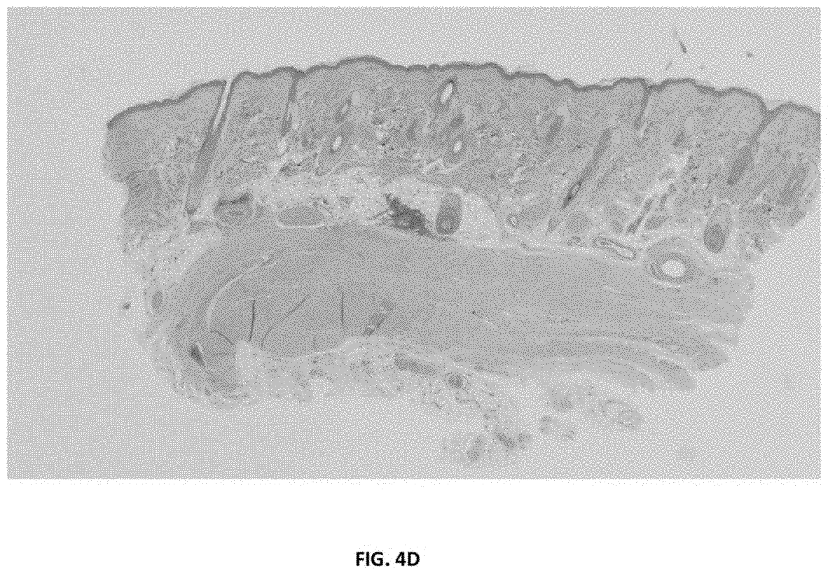

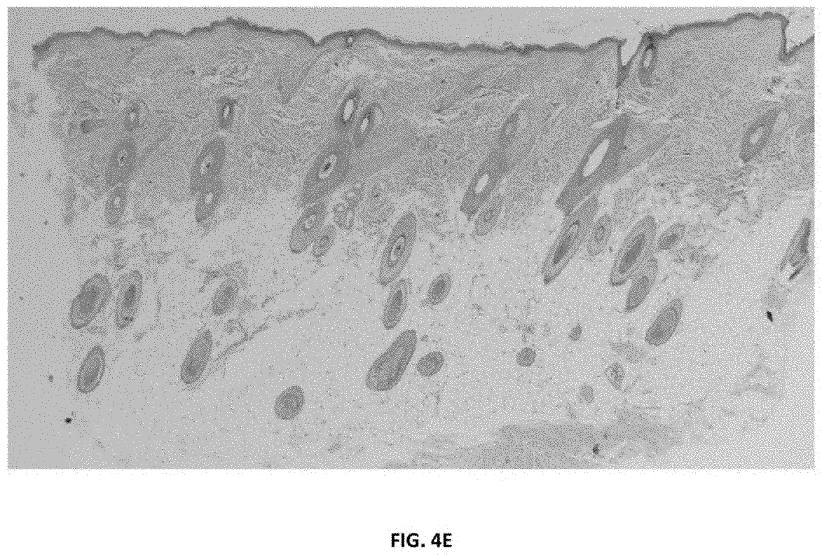

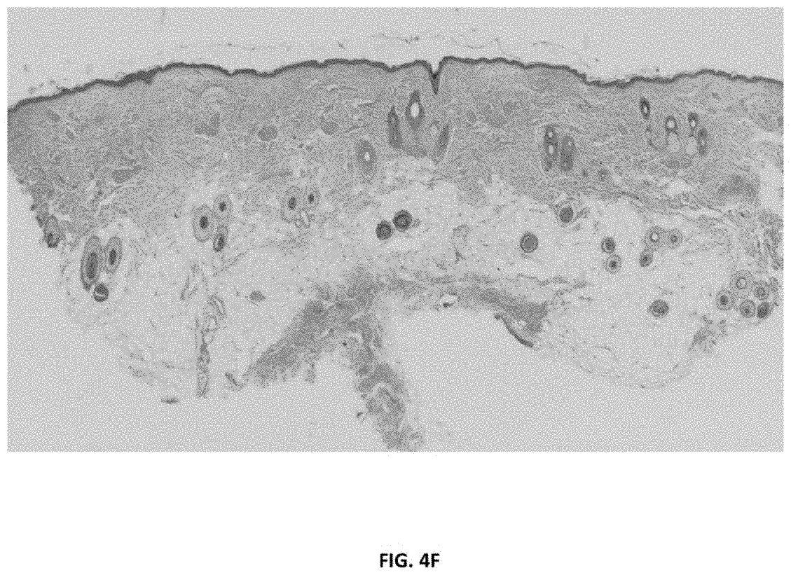

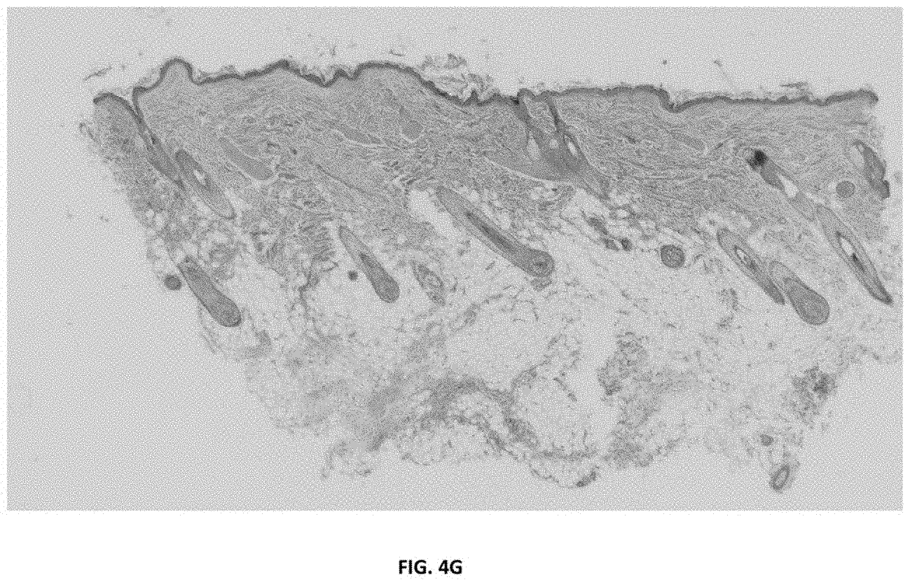

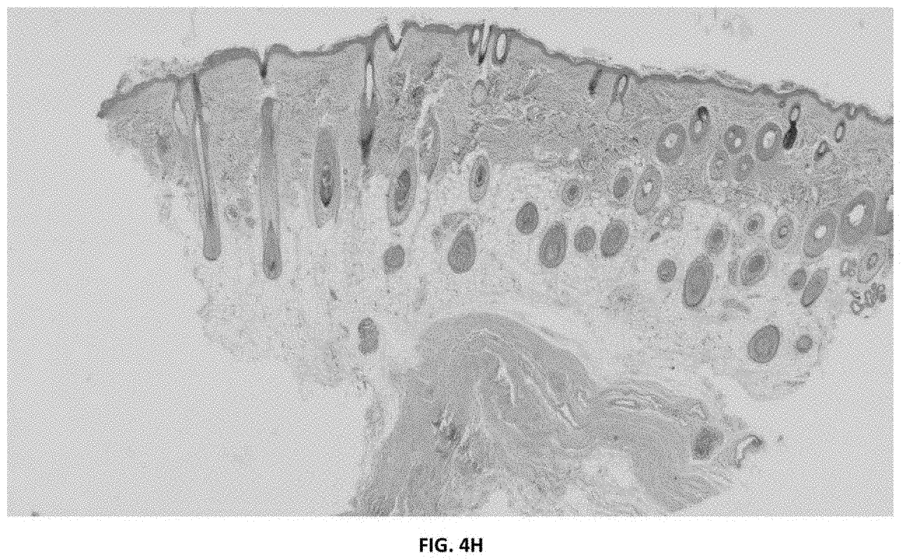

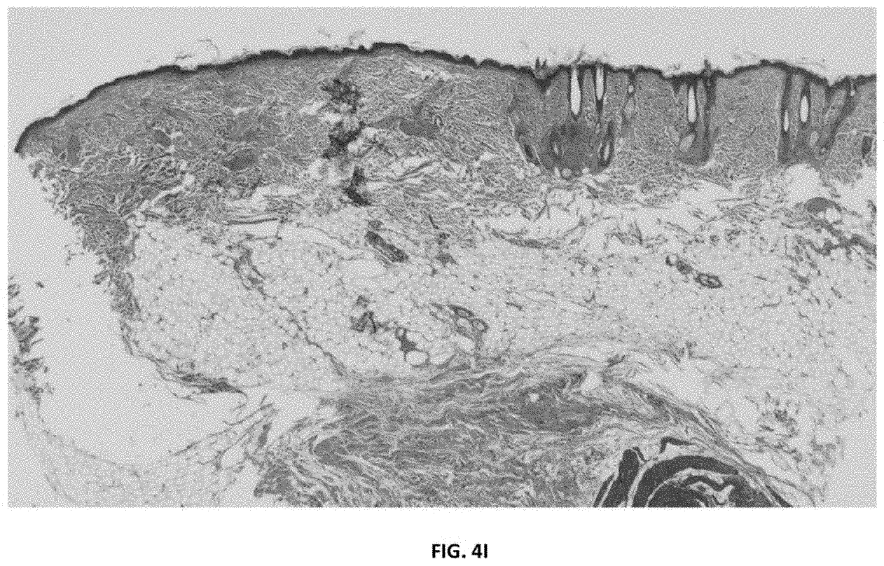

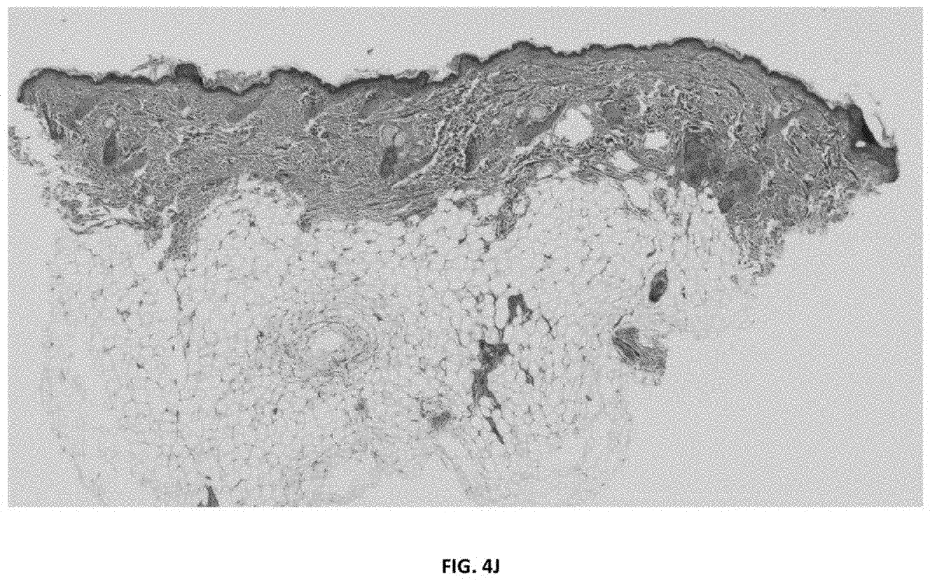

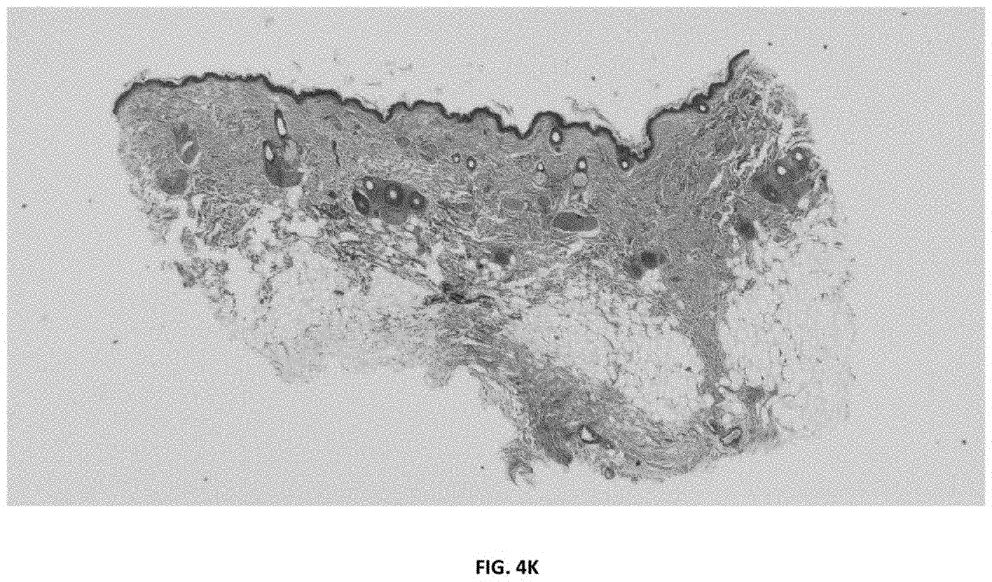

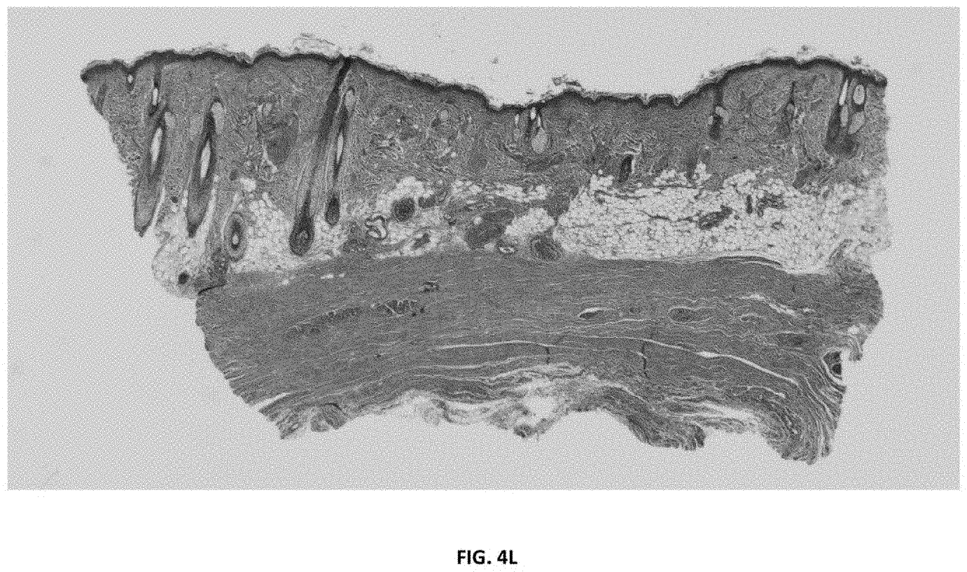

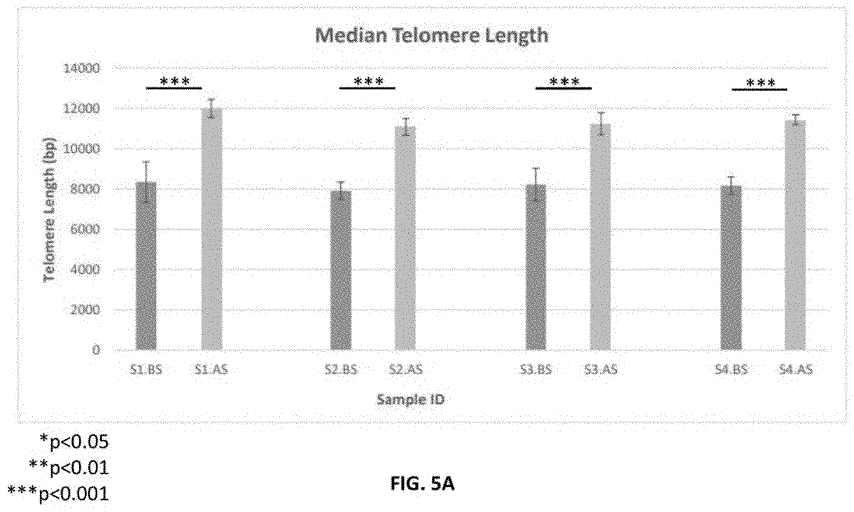

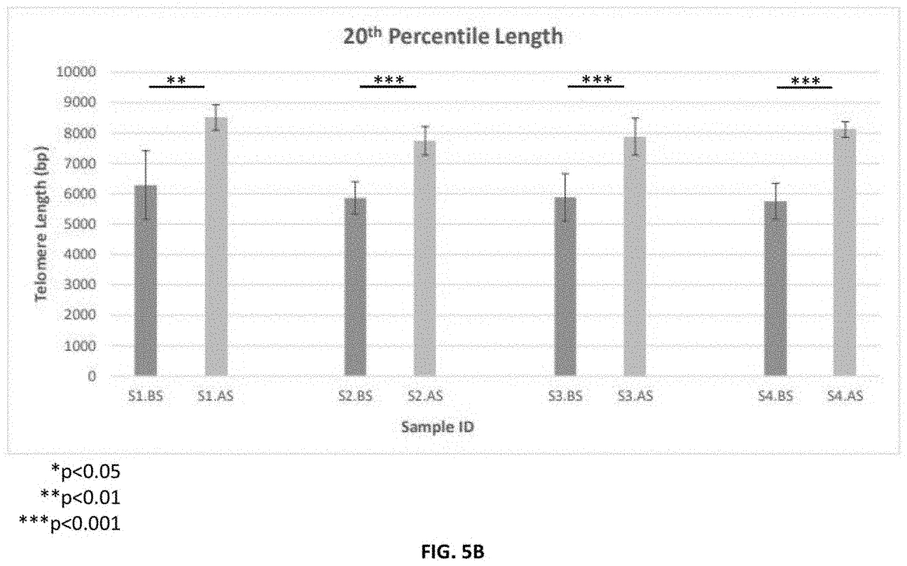

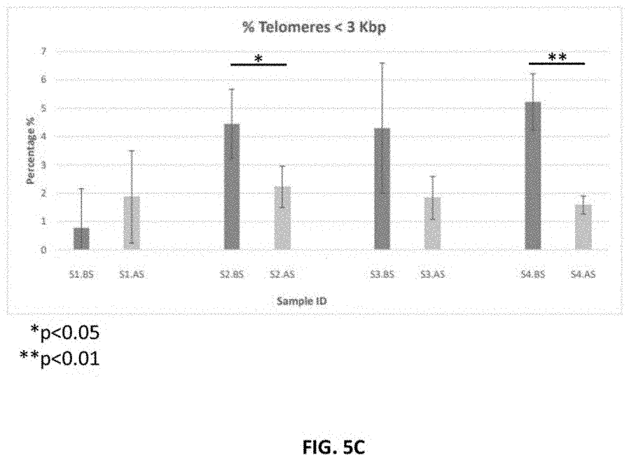

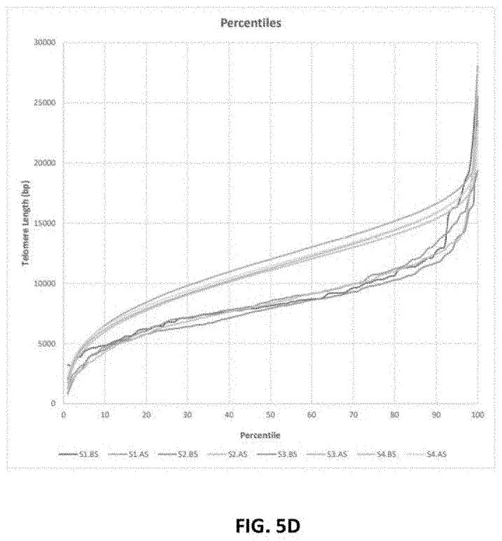

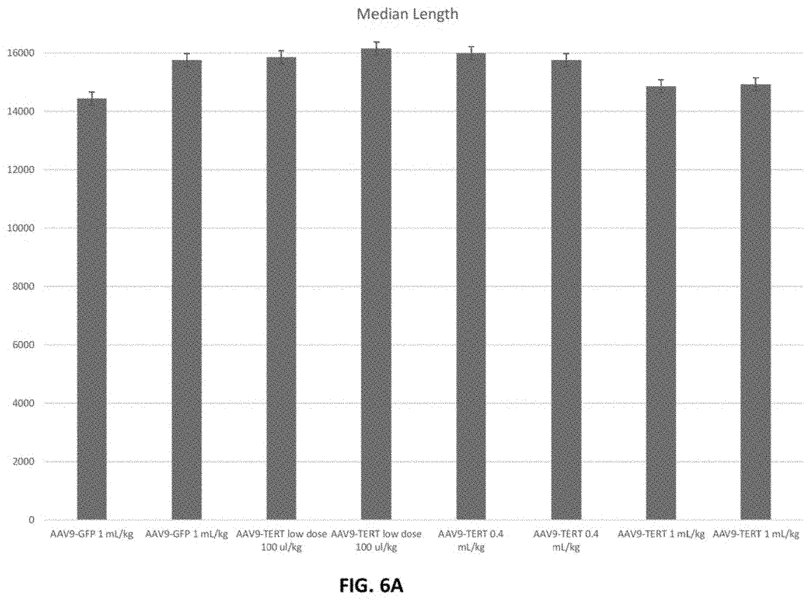

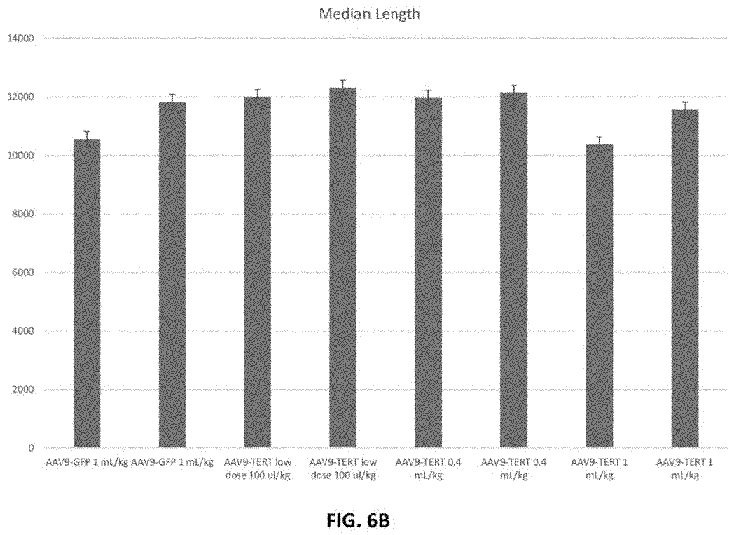

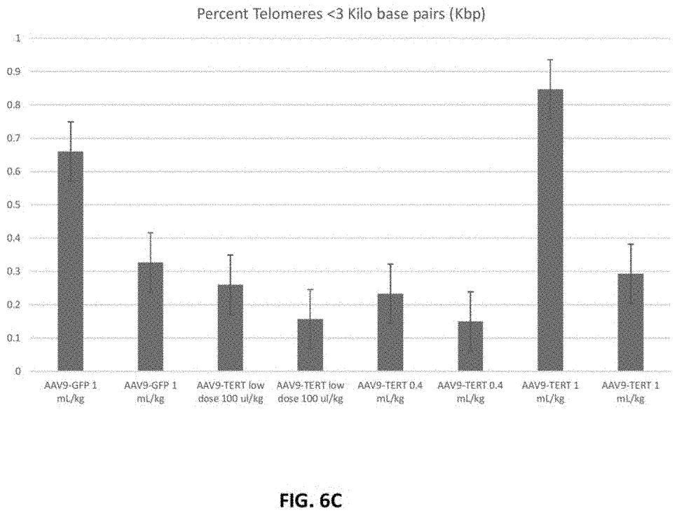

FIGS. 1A-1H shows the fluorescence of harvested organs from individual mice and after treatment with either AAV9-GFP or AAV9-RFP. FIGS. 1I-1Z shows the fluorescence of individual harvested organs from each mouse. The results of this experiments shows that all of the organs except spleen (Heart, brain, lung, liver, kidney, colon, skin, bone and muscle) showed the fluorescent expression after IV injection of virus. The sum of GFP mean density increased as the injection dose increased. The highest GFP mean density of brain, kidney, liver and skin were observed in group 2. The highest RFP mean density of bone, brain, kidney and skin were observed in group 2. The highest GFP and RFP mean density of colon, heart, and muscle were observed in group 3. The highest GFP mean density of lung was observed in group 1. The highest RFP mean density of liver and lung were observed in group 1. The highest GFP mean density of bone was observed in group 3.

The data showed that the decrease in fluorescence amongst many tissues is consistent with an immune response to the second AAV9 administration from the lower levels of RFP fluorescence detected.

### Example 2: Evaluation for In Vivo PEI-Mediated Delivery Vs Adenoviral Delivery of Polynucleotides in Rodents

### Purpose

The purpose of this experiment was to determine if PEI could be used to deliver a polynucleotide in vivo in rodents.

### Methods

An initial study was performed to assess whether branched nchedpolyethylenimine (PEI) could be used as a delivery vector in vivo, whether 3- or 6-days post-transfection was better to assess gene delivery, whether multiple deliveries could be performed on a single animal, and what the delivery profile looked like in comparison to the adenoviral delivery method performed previously. The number of plasmids, the protein encoded, and the dose volume were selected to match previous work performed on this project as closely as possible. Two different ratios of PEI to DNA (N:P) were investigated, 7.5:1 and 5:1.

### Results

The results of the first study showed that the mice tolerated two doses of the gene delivery complexes without obvious problems. There were no deaths, and it is assumed that the mice did not lose significant weight or become cachectic. Immunological response was also tested for and found to be negative. Assessing reporter expression 6 days post-transfection was better that assessing 3-days post-transfection. Whole-organ imaging showed expression levels below what was seen with adenovirally mediated gene delivery. This was expected; however, the animals responded well to multiple deliveries using the PEI vector.

### Example 3: Evaluation for In Vivo PEI-Mediated Delivery of Polynucleotides in Rodents

### Purpose

To assess different dosages of PEI-mediated gene delivery. The N:P ratio was selected to be 5:1 based on the study described in Example 3, which did not show lower expression levels for the lower ratio. This study incorporated these findings for the evaluation of optimal dose of pAAV-CMV-PEI-GFP in Balb/c mice.

### Methods

A total of 8 Balb/c mice (seven treated, one nontreated), female, 7 weeks old, were used in the study. All animals were weighed using an electronic balance, Scout Pro 202 (OHAUS Corporation, Parsippany, NJ, U.S.A.) and given a clinical examination to ensure that they were in good condition. Treated mice were housed 1-2 per cage (Table I). An inspection was performed to ensure their suitability for the study before viral injection. The animals were maintained in a HEPA-filtered environment in a Micro-VENT full, ventilation rodent housing system (Allentown Caging Equipment Co., Allentown, NJ, U.S.A) at AntiCancer, Inc. Animal room controls were set to maintain temperature and relative humidity at 22° C.±2° C. and 55%±15%, respectively. The rooms were lit by artificial light for 12 hours each day.

### Study Compounds and Drug Preparation

Normal saline, 0.9% (155 mM), PEI is weight-average 25,000, 4.307 ug/ul (diluted in normal saline), pH 7.0, 0.22 um filtered. Plasmid assumed to be pAA V-CMV-eGFP, 4815 bp, 1.8 ug/ul, purity (A260/A280)≥1.80.

### Study Design

A total of 8 mice were divided into the 5 groups indicated above. Each mouse was injected with 400 ul pAAV-PEI-GFP Plasmid of the appropriate transfection mixture (Table 4). After 5 days, sacrifice the mice and harvest the organs: brain, eye, heart, lung, liver, spleen, kidney, bone, skin, and colon.

TABLE 4
Groups and Injection Volume

| | To mimic | # mice | Saline | PEI | | Saline | Plasmid | | Tot Vol |
|---|---|---|---|---|---|---|---|---|---|
| | AAV dose: | to treat | (ul) | (ul) | Total | (ul) | (ul) | Total | (ul) |
| G1 | (Neg. Control) | 1 | 0 | 0 | 0 | 408 | 0 | 408 | 408 |
| G2 | .400 × 10\hat{ }13 | 1 | 200.74 | 3.26 | 204 | 192.84 | 11.16 | 204 | 408 |
| G3 | .200 × 10\hat{ }14 | 2 | 375.38 | 32.62 | 408 | 296.37 | 111.63 | 408 | 816 |
| G4 | .300 × 10\hat{ }14 | 2 | 359.07 | 48.93 | 408 | 240.96 | 167.44 | 408 | 816 |
| G5 | .400 × 10\hat{ }14 | 2 | 342.76 | 65.24 | 408 | 184.75 | 223.25 | 408 | 816 |

### Mouse Weights

The weights of each mouse was recorded on the day of injection and the last day of the study. (Table 5).

TABLE 5
Mouse weights.

| Body | | | | |
|---|---|---|---|---|
| Weight | | | | |
| (g) | | | Aug. 11, 2021 | Aug. 16, 2021 |
| G1 | C1-1 | Neg. control | 18.4 | 18.5 |
| G2 | C1-2 | .400 × 10\hat{ }13 | 19.7 | 19.7 |
| G3 | C1-3 | .200 × 10\hat{ }14 | 19.8 | 19.9 |
| G3 | C1-4 | .200 × 10\hat{ }14 | 17.2 | 17.4 |
| G4 | C2-1 | .300 × 10\hat{ }14 | 18.2 | 18.2 |
| G3 | C2-2 | .300 × 10\hat{ }14 | 19.3 | 19.4 |
| G5 | C3-3 | .400 × 10\hat{ }14 | 15.7 | 15.5 |
| G5 | C2-4 | .400 × 10\hat{ }14 | 18.3 | (8/12 death) |

### Organ Harvest

On August 16^{th}, all mice were sacrificed and 10 organs (heart, brain, right eye left, lung, liver, spleen, kidney colon, skin, and bone) were harvested. Frozen section slide images were obtained using a U-TBI90 microscope (OLYMPUS, Tokyo, Japan) and PictureFrame Application 2.3 (Optronics). The mean fluorescence intensity of frozen section slides was determined using the UVP ChemStudio (Analytik Jena Co. Jena, Germany).

### Results

The mean fluorescence intensity of frozen section slides are shown in Table 6.

TABLE 6
Mean fluorescence intensity (Raw data)

| | | | | | | Rt. | | | | Lt. | |
|---|---|---|---|---|---|---|---|---|---|---|---|
| | | Bone | Brain | Colon | Ear | Eye | Heart | Kidney | Liver | Lung | Spleen |
| Group 1 | C1-1 | 27.7 | 0 | 0.3 | 8.9 | 2.4 | 6.5 | 0.2 | 15.1 | 7.1 | 6 |
| Group 2 | C1-2 | 24.9 | 4 | 3.3 | 7.1 | 40.7 | 8.4 | −0.4 | 25.1 | 12.8 | 19.3 |
| Group 3 | C1-3 | 19.5 | 11.7 | 12.9 | 62 | 27.3 | 21.9 | 14.4 | 37.5 | 11 | 27.4 |
| | C1-4 | 39.3 | 3.7 | 13.4 | 20.3 | 10.1 | 12 | 19.3 | 16.2 | 7.1 | 11.6 |
| Group 4 | C2-1 | 24.3 | 15.6 | 6.3 | 12.6 | 9.6 | 16.3 | 17.3 | 28.3 | 14.1 | 23 |
| | C2-2 | 55.1 | 9.1 | 13 | 61.7 | 32.2 | 10.9 | 18.2 | 37.8 | 19.8 | 21.9 |
| Group 5 | C2-3 | 149.7 | 1.4 | 5.4 | 49.9 | 8.3 | 8.8 | 7.7 | 19.4 | 9 | 32.2 |

The Average fluorescence intensity, minus average intensity of negative control is shown in Table 7 and Table 8.

TABLE 7
Mean fluorescence intensity (Negative control correction)

| | | | | | | Rt. | | | | Lt. | |
|---|---|---|---|---|---|---|---|---|---|---|---|
| | | Bone | Brain | Colon | Ear | Eye | Heart | Kidney | Liver | Lung | Spleen |
| Group 1 | C1-1 | 0 | 0 | 0 | 0 | 0 | 0 | 0 | 0 | 0 | 0 |
| Group 2 | C1-2 | −2.8 | 4 | 3 | −1.8 | 38.3 | 1.9 | −0.6 | 10 | 5.7 | 13.3 |
| Group 3 | C1-3 | −8.2 | 11.7 | 12.6 | 53.1 | 24.9 | 15.4 | 14.2 | 22.4 | 3.9 | 21.4 |
| | C1-4 | 11.6 | 3.7 | 13.1 | 11.4 | 7.7 | 5.5 | 19.1 | 1.1 | 0 | 5.6 |
| Group 4 | C2-1 | −3.4 | 15.6 | 6 | 3.7 | 7.2 | 9.8 | 17.1 | 13.2 | 7 | 17 |
| | C2-2 | 27.4 | 9.1 | 12.7 | 52.8 | 29.8 | 4.4 | 18 | 22.7 | 12.7 | 15.9 |
| Group 5 | C2-3 | 122 | 1.4 | 5.1 | 41 | 5.9 | 2.3 | 7.5 | 4.3 | 1.9 | 26.2 |

TABLE 8
Mean fluorescence intensity (Group 3 and Group 4: mean)

| | | | | | Rt. | | | | Lt. | |
|---|---|---|---|---|---|---|---|---|---|---|
| | Bone | Brain | Colon | Ear | Eye | Heart | Kidney | Liver | Lung | Spleen |
| Group 3 | 1.7 | 7.7 | 12.85 | 32.25 | 16.3 | 10.45 | 16.65 | 11.75 | 1.95 | 13.5 |
| Group 4 | 12 | 12.35 | 9.35 | 28.25 | 18.5 | 7.1 | 17.55 | 17.95 | 9.85 | 16.45 |

The results showed that there was a qualitative difference in the amount of expression versus the number of PEI/DNA complexes delivered. Statistical analyses could not be performed because of the number of animals in each group, numbers which were selected by the sponsor based, in part, upon cost. Relative to the 0.4×10¹² benchmark, 0.2×10¹³ and 0.3×10¹³ plasmids per dose yielded better expression. The highest dosage tested yielded death in one of the two animals in that group. Between the groups receiving 0.2×10¹³ and 0.3×10¹³ plasmids per dose, the 0.3×10¹³ group displayed a more consistent amount of expression between the group members.

The dose in Group 2 (0.4×10¹⁴) had insufficient expression of GFP. For the doses in Group 3 (0.2×10¹⁴) and Group 4 (0.3×10¹⁴), the expression of GFP was positive in most of the organs measured. In group 3, the mean expression of GFP in bone and lung was lower than that in group 4. Furthermore, all organs other than bone were positive in group 4 (FIG. 1H). Therefore, the dose of Group 4 was considered to be the optimal volume. The dose of Group 5 (0.4×10¹⁴) was not recommended because one of two mice died the day after administration of the pAAV-CMV-PEI-GFP.

Of note, there was GFP (and RFP) expression in the brain for the mice in the first study, and in many of the mice in the second study (especially the 0.3×10¹³ group). This was not expected because of the blood-brain barrier. Also of note was the amount of expression observed in the bone samples: all of the mice in Example 3 had very good expression in the bone. In this study, the expression in bone that correlated with the number of PEI/DNA complexes delivered. There was also a significant jump in bone expression (˜10-fold) in the surviving LD50 mouse; expression data were not obtained for the non-surviving mouse.

### Example 4: Evaluation for In Vitro Expression of Polynucleotides in Rodents

### Purpose

The purpose of this study was to determine if the hOSK plasmid expresses each factor in a mammalian cell line.

### Methods

The hOSK plasmid is shown in FIG. 3A (SEQ ID NO. 10). N2a cells were plated in 12-well cell culture plates. Each well of N2a cells were transfected with 1 μg of either hOSK plasmids or control plasmids (dCas9), using PEI as the delivery vehicle according to the PEI protocol. 2 days after transfection, the N2a cells were treated with 1 μg/ml Doxycycline for 1 day. Then, RNA was extracted from the cells using standard Trizol method. The RNA was then converted into cDNA, using The Maxima H Minus First Strand cDNA Synthesis Kit. cDNA and realtime qPCR was used to detect gene expressions of OCT4, SOX2 and KLF4, which were expected to have increased expression in N2a cells treated with hOSK plasmid.

### Results

qPCR analysis was performed using 4 control samples and 6 treatment samples with Gapdh as the reference gene. The expression of OCT4 (FIG. 3B), SOX2 (FIG. 3C) and KLF4 (FIG. 3D) were all significantly increased in cells transfected with the hOSK plasmids as expected. OCT4 was increased by 67-fold, SOX2 by 29-fold and KLF4 by 2-fold (Table 9). The data verified the hOSK plasmid and showed that PEI 25K was good for plasmid transfection in vitro.

TABLE 9
Expression value of OSK after deltaCT analysis
with Gapdh as the reference gene

| | OCT4 | Sox2 | KLF4 |
|---|---|---|---|
| OSK-1 | 0.168851 | 0.266548 | 0.28349 |
| OSK-2 | 0.205068 | 0.323077 | 0.340712 |
| OSK-3 | 0.108879 | 0.144037 | 0.255327 |
| OSK-4 | 0.074484 | 0.180678 | 0.251391 |
| OSK-5 | 0.12911 | 0.232918 | 0.248771 |
| OSK-6 | 0.110658 | 0.229796 | 0.200355 |
| Ctrl-1 | 0.002625 | 0.009788 | 0.129565 |
| Ctrl-2 | 0.002302 | 0.009608 | 0.159396 |
| Ctrl-3 | 0.001966 | 0.005628 | 0.1289 |
| Ctrl-4 | 0.000988 | 0.006341 | 0.071519 |
| OSK Average | 0.132842 | 0.229509 | 0.263341 |
| Ctrl Average | 0.00197 | 0.007841 | 0.122345 |
| FC | 67.42242 | 29.27005 | 2.152448 |
| P-value | 0.000596 | 0.000125 | 0.00961 |

### Example 5: Protocol for Administration of Inducible Yamanaka Factors in Non-Human Primates (NHP)

### Purpose:

The purpose of this study is to determine the optimum dosage and schedule of doxycycline to administer to the Rhesus monkeys' non-human primates (NHP) to regenerate tissue to a more youthful state. In prior studies, the product comprised hTERT delivered by an Adeno-Associated Virus serotype 9, AAV9. Three problems were encountered with this delivery vehicle.

The first is the immune response. We found with repeated injections in mice that the effectiveness of the product in the second injection was reduced to in general 30% of the Initial injection. Since the AAV is based on common viruses, a very high percentage of the population has neutralizing antibodies (NAB). This makes the product ineffective unless the patient is given immunosuppressants.

The second is the extremely high cost of the AAV. This alone would put therapies out of reach for 95% of the population in developed countries and higher in less developed countries.

The third problem encountered is the difficulty in handling. The product must be kept at −80° C. and only thawed before injection. This can be difficult for shipping and storage especially as the therapy moves from research to widespread therapies.

Various non-viral delivery systems were investigated and Polyethylenimine (PEI) was selected for further studies for solving the aforementioned problems.

The mouse studies using repeated injections show no detectable immune response, i.e., no need for NAB testing or rejecting a very high percentage of the population based on high NAB's to the AAV. When the final dosage is determined, it is ˜1-5% cost of AAV. The PEI is readily available, economical, stable, and can be kept at room temperature out of the sunlight for a month and much longer if kept in a normal household refrigerator. PEI is available locally in most countries through Sigma-Aldrich, a division of Merck.

### Methods

Study subjects consisted of (4) non-human primates (Rhesus monkeys) all over 20 years old, with room for additional monkeys if needed. The following steps occurred prior to the commencement of the study:
1) Monkeys were weighted.
2) (2) 3 ml samples of blood were taken from each monkey, label, and date each vial at the beginning of the study.
3) (2) 200 mg full dermal skin samples were taken from each monkey, label, and date at the beginning of the study. Use jackets or another form of protection to prevent scratching.
4) One 3 ml of blood is for the Zymo methylation age marker:
   i. Add to solution in the prepared vials.
   ii. Shake well then label each vial with the date and the tattoo number of the monkey.
   iii. Refrigerate −4° C. until the second blood sample is collected at the end of the study dated and labeled.
5) Take (1) further blood sample from each monkey to extract DNA to keep on file in case needed for further study. Label and date each sample with the monkeys' tattoo number.
6) Take (1) skin sample of 200 mg to be preserved in para-formaldehyde and stored at room temperature to send to Life Length with the end of study for telomere measurement. Label and date each container with the number of the monkeys' tattoo.
7) Take one more full dermal skin samples of 200 mg from each monkey and store in −80 C for further study as needed. Label and date each sample with the number of the monkeys' tattoo.

A weekly report was compiled, which included a photo of each monkey to record the visual health of each monkey. Any adverse reactions were noted on the protocol. If adverse reactions were noted, the protocol would be discontinued until the monkey is observed to return to good health. The protocol would then continue.

The following protocol was used to prepare the polynucleotide that encodes the Yamanaka Factors for IV injection:
1) 100 ml of PEI was purchased from Sigma-Aldrich through Merck in HCMC
2) The PEI was prepared per the following protocol:
   i. Starting with PEI, weight-average molecular weight=25,000 kDa, was diluted to 4.307 mg/ml using normal saline (0.9%).
   ii. Titrate to pH=7.0 with concentrated HCl, then filter with a 0.22 mm cellulose acetate filter. The amount of PEI stock solution to use per dose is dependent on the amount of plasmid to be delivered. A 5:1 (PEI) nitrogen to (DNA) phosphate ratio was used. The appropriate amount of PEI was diluted so that the PEI and DNA solutions to be used for a single dose had equivalent volumes. The polymer solution was added to the plasmid solution and mixed via pipetting up and down.
3) The plasmids were mixed with the PEI and saline solution at the following dosage in the provided containers.
4) Aged monkeys (around 25 years of age) were then prepared for intravenous drip application of the product as follows:
   Induction anesthesia: Sedate rhesus macaque by intramuscular injection of ketamine (10-12 mg/kg) plus xylazine (2 mg/kg) on the day of surgery plus Atropine (0.05 mg/kg).
   Maintenance anesthesia: 1-2% isoflurane delivered with oxygen at 1-2 L/min in a an open (non-rebreathing) system.
   Peri-operative analgesia: While closing local analgesic/incision Ropivacaine 4 mg/kg IM/SC or Bupivicaine 2.5 mg/kg IM. Meloxicam 0.2 mg/kg SC (NSAID)
   Post-operative analgesia: Inject Buprenorphine (Buprenex) 0.01 mg/kg IM every 6 to 8 hours post-surgery on first day and then twice a day (BID) for two days following surgery and then as needed
   Induction of Anesthesia: Intramuscular injection of Ketamine, Xylazine and Atropine (dosages listed above) is used to sedate the animal and induce anesthesia. If necessary, the animal would be masked with Isoflurane/02 to induce deeper anesthesia to facilitate intubation. The animal is then intubated with an appropriately sized, cuffed endotracheal tube under laryngoscopic observation. Sterile petrolatum ophthalmic ointment is placed on the cornea of the eyes to prevent drying.
5) Fill the IV drip bags (provided) with 80 ml of the mixed solution of product, PEI and saline solution per instructional video.
6) The monkeys recovered for 2 days
7) After 2 days, the monkeys began the following doxycycline protocol:
   a) Monkey #1 received a dosage of 200 mg of doxycycline each day for 2 days per week and water only for the remaining 5 days. This cycle continued for 12 weeks.
   b) Monkey #2 received a dosage 300 mg of doxycycline each day for 2 days per week and water only for the remaining 5 days. This cycle continued for 12 weeks.
   c) Monkey #3 received a dosage of 400 mg of doxycycline each day for 2 days per week and water only for the remaining 5 days. This cycle continued for 12 weeks.
   d) Monkey #4 received a dosage of 500 mg of doxycycline each day for 2 days per week and water only for the remaining 5 days. This cycle continued for 12 weeks.
8) At the end of the 12th week, 3 ml of blood was drawn for the Biomedic age (methylation) markers and sent for analysis. Each sample was labeled and dated with the monkey's tattoo number.
9) Next, a second blood sample was drawn for extraction of DNA per Polyvac's protocol for telomere analysis. Each sample was labeled and dated with the monkey's tattoo number.
10) A full dermal skin sample was also taken from each monkey, labeled, dated and freeze at −80° C.
11) A second skin sample was taken and placed in a container of para-formaldehyde. Each sample was labeled with the monkey's tattoo number and sent to Life Length for telomere measurement, along with the first skin sample in paraformaldehyde from the beginning of the study.

### Results:

Clinical observations of the four monkeys are summarized in Table 10.

TABLE 10
Clinical Observations of Monkeys Before and After Administration of
Inducible Yamanaka Factors

| Monkey No. | Clinical Observations |
|---|---|
| S1 (Female) | The monkey's skin is ruddier, the hair is smoother, and the hair is |
| | thicker compared to the beginning of the study. |
| S2 (Female) | The monkey's skin is ruddier, the hair is smoother, and the hair is |
| | thicker compared to the beginning of the study. |
| | Monkey S2 is female and past the age where they menstruate; however, |
| | on Dec. 3, 2022, monkey S2 began to menstruate again. The |
| | amount of menstrual blood was low and the menstrual cycle lasted |
| | about 2 days (Mar. 12, 2022 and Apr. 12, 2022). |
| S3 (Female) | The level of rejuvenation of monkey number 3 seems to be the most |
| | compared to the rest: the skin is ruddier, the hair is smoother, and the |
| | hair is thicker compared to the beginning of the study. |
| S4 (Female) | The skin and hair of the monkeys did not change as much compared to |
| | the beginning of the study. The monkey is still humpback, the skin is |
| | not as rosy, and the bald spot on the right side of the head also don't |
| | have much new hair growth. |

The clinical observations from this study indicate that the composition and methods of the disclosure increase the appearance of the skin and hair. Based on these results, evaluation of alopecia indicators and telomere length was performed, and methylation status will be performed.

### Example 6: Evaluation of Alopecia Indicators after In Vivo Induction of Yamanaka Factors in Non-Human Primates

### Purpose:

The purpose of this study was to evaluate samples for the incidence and severity of alopecia indicators after in vivo induction of Yamanaka Factors in non-human primates using skin samples from Example 5.

### Methods:

Twelve blinded hematoxylin and eosin (H&E)-stained glass slides were evaluated by a board-certified veterinary pathologist. The twelve samples comprise four samples taken from each NHP before the study (S1-1, S2-1, S3-1, and S4-1), four samples taken from each NHP immediately after completing the doxycycline protocol in Example 4 (S1-2, S2-2, S3-2, and S4-2), and four samples taken from each NHP seven months after completing the doxycycline protocol in Example 5 (S1-3, S2-3, S3-3, and S4-3). Tissues were evaluated for evidence of microscopic findings, pathological or not, and including background findings for non-human primates. Observations were assessed on a severity scale of 0-5: 0=absent, 1=minimal, 2=mild, 3=moderate, 4=marked, and 5=severe. Some findings may by noted as present (P) or absent (0). Histologic changes were described, wherever possible, according to their distribution, severity, and morphologic character.

### Results:

FIGS. 4A-4L shows hematoxylin and eosin (H&E) stained NHP skin section at 20× magnification. The blinded evaluations are shown in Table 11 and results for individual subjects are shown in Table 12, Table 13, and Table 14. Samples S1-1, S2-1, S1-3 and S2-3 showed marked adnexal atrophy, characterized by a loss of hair follicles in the dermis, while samples S3-1 and S3-3 showed moderate adnexal atrophy, and samples S4-1 and S4-3 showed mild adnexal atrophy. Samples S1-1 and S4-1 showed predominantly anagen-stage follicles, of those follicles present, while samples S2-1, S3-1, S1-3, S2-3 and S3-3 showed predominantly telogen-stage follicles. Catagen-stage follicles were noted, though not predominant, in samples S1-1, S3-1 and S4-1. Sample S4-3 showed a roughly even mix of anagen, catagen and telogen follicles. Samples S1-1, S2-1, S3-1, S4-1, S1-3, S2-3, S3-3 and S4-3 showed minimal mononuclear perivascular infiltrates, characterized by few lymphocytes immediately surrounding dermal vessels.

Samples S1-2, S2-2 and S4-2 showed no adnexal atrophy, and sample S3-2 showed mild adnexal atrophy, and all follicles were predominantly in anagen-stage. Samples S1-2, S3-2 and S4-2 showed minimal mononuclear perivascular infiltrates, and sample S2-2 showed mild mononuclear perivascular inflammatory infiltrates. Samples S1-2, S2-2, S3-2, and S4-2 all had higher densities of hair follicles compared to the other eight samples.

Samples S1-1, S2-1, S3-1, S1-3, S2-3 and S3-3 showed moderate to marked adnexal atrophy, consistent with alopecia, and were split between predominantly anagen and telogen follicle stage of the hair follicles remaining, suggesting further pause of the hair growth cycle. Samples S4-1, S3-2 and S4-3 showed mild adnexal atrophy and predominant anagen to mixed-phase follicles, while samples S1-2, S2-2 and S4-2 showed no adnexal atrophy, and predominant anagen-phase follicles. All samples showed generally comparable amounts of mononuclear cell infiltrates in the dermis.

TABLE 11
Summary of Incidence and Severity of Findings.

| | | Group | | |
|---|---|---|---|---|
| | | Blinded | Blinded | Blinded |
| | | Treatment | | |
| | | Blinded | Blinded | Blinded |
| | | Dose/Route/Frequency | | |
| | | Blinded | Blinded | Blinded |
| | | Species/Strain | | |
| | | NHP | NHP | NHP |
| | | Sex | | |
| | | Blinded | Blinded | Blinded |
| | | Time Point (days) | | |
| | | Timepoint | Timepoint | Timepoint |
| | | 1 | 2 | 3 |
| Tissue Findings/ | | Animal N: | | |
| Severity | | 4 | 4 | 4 |
| Skin | Number Evaluated | 4 | 4 | 4 |
| Within Normal | — | 0 | 0 | 0 |
| Limits | | | | |
| Atrophy, adnexal | Minimal (1) | 0 | 0 | 0 |
| | Mild (2) | 1 | 1 | 1 |
| | Moderate (3) | 1 | 0 | 1 |
| | Marked (4) | 2 | 0 | 2 |
| | Severe (5) | 0 | 0 | 0 |
| | Present (P) | 0 | 0 | 0 |
| | Total | 4 | 1 | 4 |
| Inflammatory | Minimal (1) | 4 | 3 | 4 |
| infiltrate, | Mild (2) | 0 | 1 | 0 |
| mononuclear, | Moderate (3) | 0 | 0 | 0 |
| perivascular | Marked (4) | 0 | 0 | 0 |
| | Severe (5) | 0 | 0 | 0 |
| | Present (P) | 0 | 0 | 0 |
| | Total | 4 | 4 | 4 |
| Catagen follicles | Minimal (1) | 0 | 0 | 0 |
| | Mild (2) | 0 | 0 | 0 |
| | Moderate (3) | 0 | 0 | 0 |
| | Marked (4) | 0 | 0 | 0 |
| | Severe (5) | 0 | 0 | 0 |
| | Present (P) | 3 | 0 | 1 |
| | Total | 3 | 0 | 1 |
| Telogen follicles | Minimal (1) | 0 | 0 | 0 |
| | Mild (2) | 0 | 0 | 0 |
| | Moderate (3) | 0 | 0 | 0 |
| | Marked (4) | 0 | 0 | 0 |
| | Severe (5) | 0 | 0 | 0 |
| | Present (P) | 2 | 0 | 4 |
| | Total | 2 | 0 | 4 |
| Anagen follicles | Minimal (1) | 0 | 0 | 0 |
| | Mild (2) | 0 | 0 | 0 |
| | Moderate (3) | 0 | 0 | 0 |
| | Marked (4) | 0 | 0 | 0 |
| | Severe (5) | 0 | 0 | 0 |
| | Present (P) | 2 | 4 | 1 |
| | Total | 2 | 4 | 1 |

TABLE 12
Summary of Hair Follicle Stage

| | Anagen | Catagen | Telogen |
|---|---|---|---|
| | (Growing | (Transition | (Resting |
| Monkey | Phase) | Phase) | Phase) |
| S1 | 1, 2 | | |
| S2 | 2 | | 1, 3 |
| S3 | 2 | | 1, 3 |
| S4 | 1, 2, 3 | 3 | 3 |

1 = Sample from Apr. 13, 2022 (before study)
2 = Sample from Jul. 10, 2022 (immediately after completing doxycycline protocol)
3 = Sample from Feb. 11, 2023 (7 months after completing doxycycline protocol)

TABLE 13
Summary of Adnexal Atrophy

| | Adnexal Atrophy | | | |
|---|---|---|---|---|
| Monkey | None | Mild | Moderate | Marked |
| S1 | 2 | | | 1, 3 |
| S2 | 2 | | | 1, 3 |
| S3 | | 2 | 1, 3 | |
| S4 | 2 | 1, 3 | | |

1 = Sample from Apr. 13, 2022 (before study)
2 = Sample from Jul. 10, 2022 (immediately after completing doxycycline protocol)
3 = Sample from Feb. 11, 2023 (7 months after completing doxycycline protocol)

TABLE 14
Summary of Perivascular Mononuclear Cell
Inflammatory Cell Infiltrate

| | Perivascular Mononuclear Cell | | |
|---|---|---|---|
| | Inflammatory Cell Infiltrate | | |
| Monkey | None | Minimal | Mild |
| S1 | | 1, 2, 3 | |
| S2 | | 1, 3 | 2 |
| S3 | | 1, 2, 3 | |
| S4 | | 1, 2, 3 | |

1 = Sample from Apr. 13, 2022 (before study)
2 = Sample from Jul. 10, 2022 (immediately after completing doxycycline protocol)
3 = Sample from Feb. 11, 2023 (7 months after completing doxycycline protocol)

The results of this study demonstrated that adnexal atrophy and hair follicle stages were improved in all subjects immediately after completion of the doxycycline protocol. A much higher density of anagen follicles was also observed in all subjects. A return to telogen follicle stage is further consistent with clinical observations 7 months after completion of the doxycycline protocol.

### Example 7: Evaluation of Telomere Length after In Vivo Induction of Yamanaka Factors in Non-Human Primates

### Purpose:

The purpose of this study was to evaluate samples for telomere length after in vivo induction of Yamanaka Factors in non-human primates using skin samples from Example 5.

### Methods:

Telomere length was performed by at the Life Length Facility. For the measurement of the median telomere length of any cell line, Life Length (LL) uses its patented high-throughput (HT) Q-FISH technique. This method is based on a quantitative fluorescence in-situ hybridization method modified for cells in interphase. In brief, telomeres were hybridized with a fluorescent Peptide Nucleic Acid probe (PNA) that recognizes three telomere repeats (sequence: Alexa488-OO-CCCTAACCCTAACCCTAA, Panagene). The images of the nuclei and telomeres were captured by a high-content screen system (see below). The intensity of the fluorescent signal from the telomeric PNA probes that hybridize to a given telomere was proportional to the length of that telomere. The intensities of fluorescence are translated to base pairs through a standard regression curve which was generated using control cell lines with known telomere length.

Sample Preparation and HT Q-FISH: On processing day, the samples and control cell lines frozen in liquid nitrogen were thawed at 37° C. and cell counts and cellular viability were determined. Cells were seeded in clear bottom black-walled 384-well plates at the density of 15,000 cells per well with 5 replicates of each sample and 8 replicates of each control cell line. Cells were fixed with methanol/acetic acid (3/1, vol/vol). Once these cells have fixed onto the plate, they were treated with pepsin to digest the cytoplasm and the nuclei were processed for in situ hybridization with the PNA probe. After several washing steps following standard DAPI incubation for DNA staining, the wells were filled up with mounting medium and the plate was stored overnight at 4° C.

HT Microscopy: Quantitative image acquisition and analysis was performed on a High Content Screening Opera Phenix System (Perkin Elmer), using the Columbus software, Version 2.9 (Perkin Elmer). Images were captured, using a 40×0.95 NA water immersion objective. UV and 488 nm excitation wavelengths were used to detect the DAPI and A488 signals respectively. With constant exposure settings, 15 independent images were captured at different positions for each well. Next, the nuclei images are used to define the region of interest for each cell, measuring telomere fluorescence intensity of the A488 image in all of them. The results of intensity for each foci were exported to the Columbus 2.4 software (Perkin Elmer). Telomere length distribution and median telomere length were calculated with Life Length's proprietary algorithms. Statistical analysis of the data was performed using T-Student test.

The TAT technology was validated for the following parameters:
A. Accuracy: The establishment of a correspondence between TAT fluorescence intensity values and telomere length measurements was achieved by performing TRF (Terminal Restriction Fragmentation) in six human lymphocyte cell lines (Calibration/Method Comparison). The same set of samples was analyzed both by TAT and by the TRF reference method (Definition of TAT Systemic Error). VALIDATION DATA showed a correlation of 0.99.
B. Precision: Serial analysis of the median telomere length values was performed on a human lymphocyte sample in different runs, days and plate positions in order to define TAT Random Error parameters (Standard Deviation, Variance).
C. Limit of Detection and Specificity: Definition of image analysis algorithms and protocol setting exist that define the lowest significant spot intensities and avoid interference by nonspecific fluorescence signals.
D. Median Reportable Range: Analysis of median telomere length of 6 cell lines is performed that covers our reportable range and defines its lower and upper limits. VALIDATION DATA fix lower level at 4,700 base pairs and upper level at 14,400 base pairs.
E. Reference Range: Analysis of median telomere length have been conducted in hundreds of human samples in order to define the TAT Reference Range and its percentiles (5th, 10th, 25th, 50th, 75th and 95th) for different ages. VALIDATION DATA established population curves—normal population data base from 18 to 85 years, to extrapolate patients' data and generate reports.

Previous to plating and before conducting the TAT protocol, the samples were assessed for:
A. Cell count: An automated cell counter is used to determine the total number of cells in the vials.
B. Cell viability by Tripan-Blue exclusion method.
C. Regression Curve: Internal controls are included, and a regression analysis is performed for each run/plate. The plates are repeated if their regression curves have an R2 below 0.92.
D. Imaging analysis filters: Data are filter-Homogenized after imaging analysis discarding spurious data, outliers and no-representative images of the sample.
E. Replicates: After seeding and once TAT is completed:
   a. Samples should have a Coefficient of Variation (CV) below 10%.
   b. Samples with less than 3 valid replicates at the end of the analysis are discarded.
   c. Spot number analyzed per sample should be higher than 10,000.

### Results:

The following table shows the median telomere length (MTL) (FIG. 5A) and 20th percentile median telomere length (both in base pairs—bp) (FIG. 5B) for each sample as well as the percentage of short telomeres. The latter is defined as the percentage of the telomeres with a length below 3 Kbp (<3 Kbp) (FIG. 5C). All measurements were performed in quintuplicate.

The extended telomere length percentiles were also measured (FIG. 5D). Percentile is the point in a distribution at which a given percentage of determined values or scores is found. The telomere length percentiles comprise of one hundred individual telomere length measurements per sample including the length of the shortest telomeres in the sample (1st percentile length) and the length of the longest telomeres (100^{th} percentile length). The percentiles allow for a comprehensive comparison between all the telomere lengths present in each sample throughout the telomere length distribution.

The results of this experiment demonstrate that median telomere length increased, 20^{th} percentile median telomere length increased, and the percentage of telomeres with a length below 3 kbp decreased after administrating the compositions and methods to the subjects. These results are in contrast to a previous study that found no significant differences in telomere length following administration of AAV9-TERT to NHP (see Example 5 and FIGS. 6A-6C).

### Example 8: Evaluation of Methylation Status after In Vivo Induction of Yamanaka Factors in Non-Human Primates

### Purpose:

The purpose of this study will be to evaluate the methylation status of samples after in vivo induction of Yamanaka Factors in non-human primates using skin samples from Example 5

### Methods

Samples from Example 5 will be subjected to methylation analysis using commercially available kits. The analysis will provide differentially methylation sites (DMS), differentially methylation regions (DMR), gene annotation, heatmap clustering, and methylation value violin plots on a global scale.

### Results

The results of this experiments are expected to demonstrate that the induction of Yamanaka Factors in NHP decreases age-dependent CpG signatures compared to baseline.

### NUMBERED EMBODIMENTS

Embodiment 1. A composition to increase cellular longevity comprising a polyethylenimine (PEI) formulation and a polynucleotide;
wherein the polynucleotide encodes:
one or more heterologous polynucleotide(s) encoding one or more Yamanaka Factors, wherein the one or more polynucleotide(s) encoding the one or more Yamanaka Factors is operably linked to a promoter that is not in nature associated with the one or more polynucleotide(s) encoding the one or more Yamanaka Factors, said promoter directly or indirectly induced;
wherein the polynucleotide expresses the polynucleotide(s) encoding one or more Yamanaka Factors under conditions that induce the promoter operably linked to the polynucleotide(s) encoding the one or more Yamanaka Factors; and
wherein the polynucleotide is bound to the cationic monomers and is substantially located within the core of the PEI formulation.

Embodiment 2. The composition of embodiment 1, wherein the one or more heterologous polynucleotide(s) encoding one or more Yamanaka Factors:
a) one or more heterologous polynucleotide(s) encoding an octamer-binding transcription factor 4 (OCT4), wherein the one or more polynucleotide(s) encoding the OCT4 is operably linked to an inducible promoter that is not in nature associated with the one or more polynucleotide(s) encoding the OCT4, said promoter directly or indirectly induced;
b) one or more heterologous polynucleotide(s) encoding a Kruppel Like Factor 4 (KLF4), wherein the one or more polynucleotide(s) encoding the KLF4 is operably linked to a promoter that is not in nature associated with the one or more polynucleotide(s) encoding the KLF4, said promoter directly or indirectly induced;
c) one or more heterologous polynucleotide(s) encoding an SRY-box 2 (SOX2), wherein the one or more polynucleotide(s) encoding the SOX2 is operably linked to an inducible promoter that is not in nature associated with the one or more polynucleotide(s) encoding the SOX2, said promoter directly or indirectly induced;
wherein the polynucleotide expresses: the polynucleotide(s) encoding OCT4 under conditions that induce the promoter operably linked to the polynucleotide(s) encoding OCT4; the polynucleotide(s) encoding KLF4 under conditions that induce the promoter operably linked to the polynucleotide(s) encoding KLF4; the polynucleotide(s) encoding SOX2 under conditions that include the promoter operably linked to the polynucleotide(s) encoding SOX2; and
wherein the polynucleotide is bound to the cationic monomers and is substantially located within the core of the PEI formulation.

Embodiment 3. The composition of embodiments 1 or 2, wherein the promoter operably linked to the polynucleotide(s) encoding OCT4, the promoter operably linked to KLF4, and the promoter operably linked to the polynucleotide(s) encoding SOX2 are the same promoter.

Embodiment 4. The composition of embodiments 1-3, wherein at least one polynucleotide is operably linked to a directly or indirectly inducible promoter that is induced by a chemical and/or nutritional inducer.

Embodiment 5. The composition of embodiments 1-4, where the chemical and/or nutritional inducer is selected from arabinose, IPTG, tetracycline, and rhamnose.

Embodiment 6. The composition of embodiments 1-5, wherein at least one polynucleotide is under the control of a Tet promoter sequence and a Tet repressor polynucleotide.

Embodiment 7. The composition of embodiments 1-6, wherein the OCT4 polynucleotide shares at least 80%, at least 85%, at least 90%, at least 95%, at least 98%, at least 99%, or 100% identity to SEQ ID NO: 1.

Embodiment 8. The composition of embodiments 1-7, wherein the polynucleotide encodes a OCT4 polypeptide shares at least 80%, at least 85%, at least 90%, at least 95%, at least 98%, at least 99%, or 100% identity to SEQ ID NO: 4.

Embodiment 9. The composition of embodiments 1-8, wherein the SOX2 polynucleotide shares at least 80%, at least 85%, at least 90%, at least 95%, at least 98%, at least 99%, or 100% identity to SEQ ID NO: 3.

Embodiment 10. The composition of embodiments 1-9, wherein the polynucleotide encodes a SOX2 polypeptide shares at least 80%, at least 85%, at least 90%, at least 95%, at least 98%, at least 99%, or 100% identity to SEQ ID NO: 6.

Embodiment 11. The composition of embodiments 1-10, wherein the KLF4 polynucleotide shares at least 80%, at least 85%, at least 90%, at least 95%, at least 98%, at least 99%, or 100% identity to SEQ ID NO: 2.

Embodiment 12. The composition of embodiments 1-11, wherein the polynucleotide encodes a KLF4 polypeptide shares at least 80%, at least 85%, at least 90%, at least 95%, at least 98%, at least 99%, or 100% identity to SEQ ID NO: 5.

Embodiment 13. The composition of embodiments 1-12, wherein the composition is administered to a cell.

Embodiment 14. The composition of embodiments 1-13, wherein the composition is administrated to a subject in need thereof.

Embodiment 15. The composition of embodiments 1-14, wherein the cell is a non-human primate cell.

Embodiment 16. The composition of embodiments 1-15, wherein the cell is a human cell.

Embodiment 17. The composition of embodiments 1-16, wherein the PEI formulation and polynucleotide are administered systemically.

Embodiment 18. The composition of embodiments 1-17, wherein the PEI formulation and polynucleotide are administered intravenously.

Embodiment 19. The composition of embodiments 1-18, wherein the PEI formulation and polynucleotide are administered in a single dose.

Embodiment 20. The composition of embodiments 1-19, wherein the PEI formulation and polynucleotide are administered in multiple doses.

Embodiment 21. The composition of embodiments 1-20, wherein the subject is administered a dosage of at least about 0.001 mg/kg to about 1000 mg/kg of doxycycline for at least 1 day per week and water only for the remaining days for at least 2 weeks.

Embodiment 22. The composition of embodiments 1-21, wherein the subject is administered a dosage of at least about 0.001 mg/kg to about 1000 mg/kg of doxycycline for at least 1 day per week and water only for the remaining days for at least 8 weeks.

Embodiment 23. The composition of embodiments 1-22, wherein the subject is administered a dosage of at least about 0.001 mg/kg to about 1000 mg/kg of doxycycline for at least 1 day per week and water only for the remaining days for at least 12 weeks.

Embodiment 24. The composition of embodiments 1-23, wherein the subject is administered a dosage of at least about 0.001 mg/kg to about 1000 mg/kg of doxycycline for at least 1 day per week and water only for the remaining days for at least 26 weeks.

Embodiment 25. The composition of embodiments 1-24, wherein the subject is administered a dosage of at least about 0.001 mg/kg to about 1000 mg/kg of doxycycline for at least 1 day per week and water only for the remaining days for at least 52 weeks.

Embodiment 26. The composition of embodiments 1-25, wherein the cell has increased CpG promoter hypermethylation compared to baseline.

Embodiment 27. The composition of embodiments 1-26, wherein the cell has increased telomere length compared to baseline.

Embodiment 28. The composition of embodiments 1-27, wherein the cell has from about 10% to about 50% increased median telomere length compared to baseline.

Embodiment 29. The composition of embodiments 1-28, wherein the cell has about 30% increased median telomere length compared to baseline.

Embodiment 30. The composition of embodiments 1-29, wherein the cell has from about 10% to about 50% increased 20^{th} percentile telomere length compared to baseline.

Embodiment 31. The composition of embodiments 1-30, wherein the cell has about 30% increased 20^{th} percentile telomere length compared to baseline.

Embodiment 32. The composition of embodiments 1-31, wherein the cell has about 30% increased 20^{th} percentile telomere length compared to baseline.

Embodiment 33. The composition of embodiments 1-32, wherein the cell has about 10% decreased telomeres 3 Kilo base pairs (Kbp) in length compared to baseline.

Embodiment 34. The composition of embodiments 1-33, wherein the composition increases the density of anagen follicles in a subject.

Embodiment 35. The composition of embodiments 1-34, wherein the composition increases the density of catagen follicles in a subject.

Embodiment 36. The composition of embodiments 1-35, wherein the composition decreases adnexal atrophy in a subject.

Embodiment 37. The composition of embodiments 1-36, wherein the composition decreases mononuclear perivascular inflammatory infiltrate in the dermis in a subject.

Embodiment 38. The composition of embodiments 1-37, wherein the polynucleotide and PEI comprise a N:P ratio.

Embodiment 39. The composition of embodiments 1-38, wherein the N:P ratio is from about 1:1 to about 10:1.

Embodiment 40. The composition of embodiments 1-39, wherein the N:P ratio is about 1:1 to about 1:10.

Embodiment 41. The composition of embodiments 1-40, wherein the N:P ratio is about 5:1.

Embodiment 42. The composition of embodiments 1-41, wherein the composition increases fertility in a subject.

Embodiment 43. The composition of embodiments 1-42, wherein the composition increases lifespan, decreases hair loss in a subject by about 10%, and/or increases skin elasticity by about 10% in a subject compared to a subject that was not administered the composition.

Embodiment 44. A method to increase cellular longevity comprising administering a polyethylenimine (PEI) formulation and a polynucleotide to a subject in need thereof;
wherein the polynucleotide encodes:
one or more heterologous polynucleotide(s) encoding one or more Yamanaka Factors, wherein the one or more polynucleotide(s) encoding the one or more Yamanaka Factors is operably linked to a promoter that is not in nature associated with the one or more polynucleotide(s) encoding the one or more Yamanaka Factors, said promoter directly or indirectly induced;
wherein the polynucleotide expresses the polynucleotide(s) encoding one or more Yamanaka Factors under conditions that induce the promoter operably linked to the polynucleotide(s) encoding the one or more Yamanaka Factors; and
wherein the polynucleotide is bound to the cationic monomers and is substantially located within the core of the PEI formulation.

Embodiment 45. The method of embodiment 44, wherein the one or more heterologous polynucleotide(s) encoding one or more Yamanaka Factors:
a) one or more heterologous polynucleotide(s) encoding an octamer-binding transcription factor 4 (OCT4), wherein the one or more polynucleotide(s) encoding the OCT4 is operably linked to an inducible promoter that is not in nature associated with the one or more polynucleotide(s) encoding the OCT4, said promoter directly or indirectly induced;
b) one or more heterologous polynucleotide(s) encoding a Kruppel Like Factor 4 (KLF4), wherein the one or more polynucleotide(s) encoding the KLF4 is operably linked to a promoter that is not in nature associated with the one or more polynucleotide(s) encoding the KLF4, said promoter directly or indirectly induced;
c) one or more heterologous polynucleotide(s) encoding an SRY-box 2 (SOX2), wherein the one or more polynucleotide(s) encoding the SOX2 is operably linked to an inducible promoter that is not in nature associated with the one or more polynucleotide(s) encoding the SOX2, said promoter directly or indirectly induced;
wherein the polynucleotide expresses: the polynucleotide(s) encoding OCT4 under conditions that induce the promoter operably linked to the polynucleotide(s) encoding OCT4; the polynucleotide(s) encoding KLF4 under conditions that induce the promoter operably linked to the polynucleotide(s) encoding KLF4; the polynucleotide(s) encoding SOX2 under conditions that include the promoter operably linked to the polynucleotide(s) encoding SOX2; and
wherein the polynucleotide is bound to the cationic monomers and is substantially located within the core of the PEI formulation.

Embodiment 46. The method of embodiments 44 or 45, wherein the promoter operably linked to the polynucleotide(s) encoding OCT4, the promoter operably linked to KLF4, and the promoter operably linked to the polynucleotide(s) encoding SOX2 are the same promoter.

Embodiment 47. The method of embodiments 44-46, wherein at least one polynucleotide is operably linked to a directly or indirectly inducible promoter that is induced by a chemical and/or nutritional inducer.

Embodiment 48. The method of embodiments 44-47, where the chemical and/or nutritional inducer is selected from arabinose, IPTG, tetracycline, and rhamnose.

Embodiment 49. The method of embodiments 44-48, wherein at least one polynucleotide is under the control of a Tet promoter sequence and a Tet repressor polynucleotide.

Embodiment 50. The method of embodiments 44-49, wherein the OCT4 polynucleotide shares at least 80%, at least 85%, at least 90%, at least 95%, at least 98%, at least 99%, or 100% identity to SEQ ID NO: 1.

Embodiment 51. The method of embodiments 44-50, wherein the polynucleotide encodes a OCT4 polypeptide shares at least 80%, at least 85%, at least 90%, at least 95%, at least 98%, at least 99%, or 100% identity to SEQ ID NO: 4.

Embodiment 52. The method of embodiments 44-51, wherein the SOX2 polynucleotide shares at least 80%, at least 85%, at least 90%, at least 95%, at least 98%, at least 99%, or 100% identity to SEQ ID NO: 3.

Embodiment 53. The method of embodiments 44-52, wherein the polynucleotide encodes a SOX2 polypeptide shares at least 80%, at least 85%, at least 90%, at least 95%, at least 98%, at least 99%, or 100% identity to SEQ ID NO: 6.

Embodiment 54. The method of embodiments 44-53, wherein the KLF4 polynucleotide shares at least 80%, at least 85%, at least 90%, at least 95%, at least 98%, at least 99%, or 100% identity to SEQ ID NO: 2.

Embodiment 55. The method of embodiments 44-54, wherein the polynucleotide encodes a KLF4 polypeptide shares at least 80%, at least 85%, at least 90%, at least 95%, at least 98%, at least 99%, or 100% identity to SEQ ID NO: 5.

Embodiment 56. The method of embodiments 44-55, wherein the PEI formulation and the polynucleotide contacts a cell of the subject.

Embodiment 57. The method of embodiments 44-56, wherein the subject is a human.

Embodiment 58. The method of embodiments 44-57, wherein the cell is a non-human primate cell.

Embodiment 59. The method of embodiments 44-58, wherein the cell is a human cell.

Embodiment 60. The method of embodiments 44-59, wherein the PEI formulation and polynucleotide are administered systemically.

Embodiment 61. The method of embodiments 44-60, wherein the PEI formulation and polynucleotide are administered intravenously.

Embodiment 62. The method of embodiments 44-61, wherein the PEI formulation and polynucleotide are administered in a single dose.

Embodiment 63. The method of embodiments 44-62, wherein the PEI formulation and polynucleotide are administered in multiple doses.

Embodiment 64. The method of embodiments 44-63, wherein the subject is administered a dosage of at least about 0.001 mg/kg to about 1000 mg/kg of doxycycline for at least 1 day per week and water only for the remaining days for at least 2 weeks.

Embodiment 65. The method of embodiments 44-4, wherein the subject is administered a dosage of at least about 0.001 mg/kg to about 1000 mg/kg of doxycycline for at least 1 day per week and water only for the remaining days for at least 8 weeks.

Embodiment 66. The method of embodiments 44-65, wherein the subject is administered a dosage of at least about 0.001 mg/kg to about 1000 mg/kg of doxycycline for at least 1 day per week and water only for the remaining days for at least 12 weeks.

Embodiment 67. The method of embodiments 44-66, wherein the subject is administered a dosage of at least about 0.001 mg/kg to about 1000 mg/kg of doxycycline for at least 1 day per week and water only for the remaining days for at least 26 weeks.

Embodiment 68. The method of embodiments 44-67, wherein the subject is administered a dosage of at least about 0.001 mg/kg to about 1000 mg/kg of doxycycline for at least 1 day per week and water only for the remaining days for at least 52 weeks.

Embodiment 69. The method of embodiments 44-68, wherein the cell has increased CpG promoter hypermethylation compared to baseline.

Embodiment 70. The method of embodiments 44-69, wherein the cell has increased telomere length compared to baseline.

Embodiment 71. The method of embodiments 44-70, wherein the cell has from about 10% to about 50% increased median telomere length compared to baseline.

Embodiment 72. The method of embodiments 44-71, wherein the cell has about 30% increased median telomere length compared to baseline.

Embodiment 73. The method of embodiments 44-72, wherein the cell has from about 10% to about 50% increased 20^{th} percentile telomere length compared to baseline.

Embodiment 74. The method of embodiments 44-73, wherein the cell has about 30% increased 20^{th} percentile telomere length compared to baseline.

Embodiment 75. The method of embodiments 44-74, wherein the cell has about 30% increased 20^{th} percentile telomere length compared to baseline.

Embodiment 76. The method of embodiments 44-75, wherein the cell has about 10% decreased telomeres 3 Kilo base pairs (Kbp) in length compared to baseline.

Embodiment 77. The method of embodiments 44-76, wherein the method increases the density of anagen follicles in a subject.

Embodiment 78. The method of embodiments 44-77, wherein the method increases the density of catagen follicles in a subject.

Embodiment 79. The method of embodiments 44-78, wherein the method decreases adnexal atrophy in a subject.

Embodiment 80. The method of embodiments 44-79, wherein the method decreases mononuclear perivascular inflammatory infiltrate in the dermis in a subject.

Embodiment 81. The method of embodiments 44-80, wherein the polynucleotide and PEI is administered in a N:P ratio.

Embodiment 82. The method of embodiments 44-81, wherein the N:P ratio is from about 1:1 to about 10:1.

Embodiment 83. The method of embodiments 44-82, wherein the N:P ratio is about 1:1 to about 1:10.

Embodiment 84. The method of embodiments 44-83, wherein the N:P ratio is about 5:1.

Embodiment 85. The method of embodiments 44-84, wherein the method increases fertility in a subject.

Embodiment 86. The method of embodiments 44-85, wherein the method increases lifespan, decreases hair loss in a subject by about 10%, and/or increases skin elasticity by about 10% in a subject compared to a subject that was not administered the method.

Embodiment 87. A formulation comprising:
a) PEI
b) A polynucleotide encoding:
   a. one or more heterologous polynucleotide(s) encoding an Octamer-binding transcription factor 4 (OCT4), wherein the one or more polynucleotide(s) encoding the OCT4 is operably linked to a promoter that is not in nature associated with the one or more polynucleotide(s) encoding the OCT4, said promoter directly or indirectly induced;
   b. one or more heterologous polynucleotide(s) encoding a Kruppel Like Factor 4 (KLF4), wherein the one or more polynucleotide(s) encoding the KLF4 is operably linked to a promoter that is not in nature associated with the one or more polynucleotide(s) encoding the KLF4, said promoter directly or indirectly induced;
   c. one or more heterologous polynucleotide(s) encoding an SRY-box 2 (SOX2), wherein the one or more polynucleotide(s) encoding the SOX2 is operably linked to an inducible promoter that is not in nature associated with the one or more polynucleotide(s) encoding the SOX2, said promoter directly or indirectly induced; wherein the polynucleotide expresses the polynucleotide(s) encoding OCT4 under conditions that induce the promoter operably linked to the polynucleotide(s) encoding OCT4; the polynucleotide(s) encoding KLF4 under conditions that induce the promoter operably linked to the polynucleotide(s) encoding KLF4; the polynucleotide(s) encoding SOX2 under conditions that include the promoter operably linked to the polynucleotide(s) encoding SOX2
c) a pharmaceutically acceptable carrier

Embodiment 88. The formulation of embodiment 87, wherein the promoter operably linked to the polynucleotide(s) encoding OCT4, the promoter operably linked to KLF4, and the promoter operably linked to the polynucleotide(s) encoding SOX2 are the same promoter.

Embodiment 89. The formulation of embodiment 87 or 88, wherein at least one polynucleotide is operably linked to a directly or indirectly inducible promoter that is induced by a chemical and/or nutritional inducer.

Embodiment 90. The formulation of embodiments 87-89, where the chemical and/or nutritional inducer is selected from arabinose, IPTG, tetracycline, and rhamnose.

Embodiment 91. The formulation of embodiments 87-90, wherein at least one polynucleotide is under the control of a Tet promoter sequence and a Tet repressor polynucleotide.

Embodiment 92. The formulation of embodiments 87-91, wherein the OCT4 polynucleotide shares at least 80%, at least 85%, at least 90%, at least 95%, at least 98%, at least 99%, or 100% identity to SEQ ID NO: 1.

Embodiment 93. The formulation of embodiments 87-92, wherein the polynucleotide encodes a OCT4 polypeptide shares at least 80%, at least 85%, at least 90%, at least 95%, at least 98%, at least 99%, or 100% identity to SEQ ID NO: 4.

Embodiment 94. The formulation of embodiments 87-93, wherein the SOX2 polynucleotide shares at least 80%, at least 85%, at least 90%, at least 95%, at least 98%, at least 99%, or 100% identity to SEQ ID NO: 3.

Embodiment 95. The formulation of embodiments 87-94, wherein the polynucleotide encodes a SOX2 polypeptide shares at least 80%, at least 85%, at least 90%, at least 95%, at least 98%, at least 99%, or 100% identity to SEQ ID NO: 6.

Embodiment 96. The formulation of embodiments 87-95, wherein the KLF4 polynucleotide shares at least 80%, at least 85%, at least 90%, at least 95%, at least 98%, at least 99%, or 100% identity to SEQ ID NO: 2.

Embodiment 97. The formulation of embodiments 87-96, wherein the polynucleotide encodes a KLF4 polypeptide shares at least 80%, at least 85%, at least 90%, at least 95%, at least 98%, at least 99%, or 100% identity to SEQ ID NO: 5.

Embodiment 98. The formulation of embodiments 87-97, wherein the formulation contacts a cell of a subject.

Embodiment 99. The formulation of embodiments 87-98, wherein the subject is a human.

Embodiment 100. The formulation of embodiments 87-99, wherein the cell is a non-human primate cell.

Embodiment 101. The formulation of embodiments 87-100, wherein the cell is a human cell.

Embodiment 102. The formulation of embodiments 87-101, wherein the PEI formulation and polynucleotide are administered systemically.

Embodiment 103. The formulation of embodiments 87-102, wherein the PEI formulation and polynucleotide are administered intravenously.

Embodiment 104. The formulation of embodiments 87-103, wherein the PEI formulation and polynucleotide are administered in a single dose.

Embodiment 105. The formulation of embodiments 87-104, wherein the PEI formulation and polynucleotide are administered in multiple doses.

Embodiment 106. The formulation of embodiments 87-105, wherein the subject is administered a dosage of at least about 0.001 mg/kg to about 1000 mg/kg of doxycycline for at least 1 day per week and water only for the remaining days for at least 2 weeks.

Embodiment 107. The formulation of embodiments 87-106, wherein the subject is administered a dosage of at least about 0.001 mg/kg to about 1000 mg/kg of doxycycline for at least 1 day per week and water only for the remaining days for at least 8 weeks.

Embodiment 108. The formulation of embodiments 87-107, wherein the subject is administered a dosage of at least about 0.001 mg/kg to about 1000 mg/kg of doxycycline for at least 1 day per week and water only for the remaining days for at least 12 weeks.

Embodiment 109. The formulation of embodiments 87-108, wherein the subject is administered a dosage of at least about 0.001 mg/kg to about 1000 mg/kg of doxycycline for at least 1 day per week and water only for the remaining days for at least 26 weeks.

Embodiment 110. The formulation of embodiments 87-109, wherein the subject is administered a dosage of at least about 0.001 mg/kg to about 1000 mg/kg of doxycycline for at least 1 day per week and water only for the remaining days for at least 52 weeks.

Embodiment 111. The formulation of embodiments 87-110, wherein the cell has increased CpG promoter hypermethylation compared to baseline.

Embodiment 112. The formulation of embodiments 87-111, wherein the cell has increased telomere length compared to baseline.

Embodiment 113. The formulation of embodiments 87-112, wherein the cell has from about 10% to about 50% increased median telomere length compared to baseline.

Embodiment 114. The formulation of embodiments 87-113, wherein the cell has about 30% increased median telomere length compared to baseline.

Embodiment 115. The formulation of embodiments 87-114, wherein the cell has from about 10% to about 50% increased 20^{th} percentile telomere length compared to baseline.

Embodiment 116. The formulation of embodiments 87-115, wherein the cell has about 30% increased 20^{th} percentile telomere length compared to baseline.

Embodiment 117. The formulation of embodiments 87-116, wherein the cell has about 30% increased 20^{th} percentile telomere length compared to baseline.

Embodiment 118. The formulation of embodiments 87-117, wherein the cell has about 10% decreased telomeres 3 Kilo base pairs (Kbp) in length compared to baseline.

Embodiment 119. The formulation of embodiments 87-118, wherein the formulation increases the density of anagen follicles in a subject.

Embodiment 120. The formulation of embodiments 87-119, wherein the formulation increases the density of catagen follicles in a subject.

Embodiment 121. The formulation of embodiments 87-120, wherein the formulation decreases adnexal atrophy in a subject.

Embodiment 122. The formulation of embodiments 87-121, wherein the formulation decreases mononuclear perivascular inflammatory infiltrate in the dermis in a subject.

Embodiment 123. The formulation of embodiments 87-122, wherein the polynucleotide and PEI is administered in a N:P ratio.

Embodiment 124. The formulation of embodiments 87-123, wherein the N:P ratio is from about 1:1 to about 10:1.

Embodiment 125. The formulation of embodiments 87-124, wherein the N:P ratio is about 1:1 to about 1:10.

Embodiment 126. The formulation of embodiments 87-125, wherein the N:P ratio is about 5:1.

Embodiment 127. The formulation of embodiments 87-126, wherein the method increases fertility in a subject.

Embodiment 128. The formulation of embodiments 87-127, wherein the method increases lifespan, decreases hair loss in a subject by about 10%, and/or increases skin elasticity by about 10% in a subject compared to a subject that was not administered the formulation.

Embodiment 129. A cell comprising a polynucleotide, said polynucleotide encoding:
one or more heterologous polynucleotide(s) encoding one or more Yamanaka Factors, wherein the one or more polynucleotide(s) encoding the one or more Yamanaka Factors is operably linked to a promoter that is not in nature associated with the one or more polynucleotide(s) encoding the one or more Yamanaka Factors, said promoter directly or indirectly induced;
wherein the polynucleotide expresses the polynucleotide(s) encoding one or more Yamanaka Factors under conditions that induce the promoter operably linked to the polynucleotide(s) encoding the one or more Yamanaka Factors.

Embodiment 130. The cell of embodiment 129, wherein the one or more heterologous polynucleotide(s) encoding one or more Yamanaka Factors:
a) one or more heterologous polynucleotide(s) encoding an octamer-binding transcription factor 4 (OCT4), wherein the one or more polynucleotide(s) encoding the OCT4 is operably linked to an inducible promoter that is not in nature associated with the one or more polynucleotide(s) encoding the OCT4, said promoter directly or indirectly induced;
b) one or more heterologous polynucleotide(s) encoding a Kruppel Like Factor 4 (KLF4), wherein the one or more polynucleotide(s) encoding the KLF4 is operably linked to a promoter that is not in nature associated with the one or more polynucleotide(s) encoding the KLF4, said promoter directly or indirectly induced;
c) one or more heterologous polynucleotide(s) encoding an SRY-box 2 (SOX2), wherein the one or more polynucleotide(s) encoding the SOX2 is operably linked to an inducible promoter that is not in nature associated with the one or more polynucleotide(s) encoding the SOX2, said promoter directly or indirectly induced;
wherein the polynucleotide expresses: the polynucleotide(s) encoding OCT4 under conditions that induce the promoter operably linked to the polynucleotide(s) encoding OCT4; the polynucleotide(s) encoding KLF4 under conditions that induce the promoter operably linked to the polynucleotide(s) encoding KLF4; the polynucleotide(s) encoding SOX2 under conditions that include the promoter operably linked to the polynucleotide(s) encoding SOX2.

Embodiment 131. The cell of embodiments 129 or 130, wherein the promoter operably linked to the polynucleotide(s) encoding OCT4, the promoter operably linked to KLF4, and the promoter operably linked to the polynucleotide(s) encoding SOX2 are the same promoter.

Embodiment 132. The cell of embodiments 129-131, wherein at least one polynucleotide is operably linked to a directly or indirectly inducible promoter that is induced by a chemical and/or nutritional inducer.

Embodiment 133. The cell of embodiments 129-132, where the chemical and/or nutritional inducer is selected from arabinose, IPTG, tetracycline, and rhamnose.

Embodiment 134. The cell of embodiments 129-133, wherein at least one polynucleotide is under the control of a Tet promoter sequence and a Tet repressor polynucleotide.

Embodiment 135. The cell of embodiments 129-134, wherein the OCT4 polynucleotide shares at least 80%, at least 85%, at least 90%, at least 95%, at least 98%, at least 99%, or 100% identity to SEQ ID NO: 1.

Embodiment 136. The cell of embodiments 129-135, wherein the polynucleotide encodes a OCT4 polypeptide shares at least 80%, at least 85%, at least 90%, at least 95%, at least 98%, at least 99%, or 100% identity to SEQ ID NO: 4.

Embodiment 137. The cell of embodiments 129-136, wherein the SOX2 polynucleotide shares at least 80%, at least 85%, at least 90%, at least 95%, at least 98%, at least 99%, or 100% identity to SEQ ID NO: 3.

Embodiment 138. The cell of embodiments 129-137, wherein the polynucleotide encodes a SOX2 polypeptide shares at least 80%, at least 85%, at least 90%, at least 95%, at least 98%, at least 99%, or 100% identity to SEQ ID NO: 6.

Embodiment 139. The cell of embodiments 129-138, wherein the KLF4 polynucleotide shares at least 80%, at least 85%, at least 90%, at least 95%, at least 98%, at least 99%, or 100% identity to SEQ ID NO: 2.

Embodiment 140. The cell of embodiments 129-139, wherein the polynucleotide encodes a KLF4 polypeptide shares at least 80%, at least 85%, at least 90%, at least 95%, at least 98%, at least 99%, or 100% identity to SEQ ID NO: 5.

Embodiment 141. The cell of embodiments 129-140, wherein the cell is a non-human primate cell.

Embodiment 142. The cell of embodiments 129-141, wherein the cell is a human cell.

Embodiment 143. The cell of embodiments 129-142, wherein doxycycline is administered to the cell.

Embodiment 144. The cell of embodiments 129-143, wherein the cell is administered a dosage of at least about 0.001 mg/kg to about 1000 mg/kg of doxycycline for at least 1 day per week for at least 2 weeks.

Embodiment 145. The cell of embodiments 129-144, wherein the cell is administered a dosage of at least about 0.001 mg/kg to about 1000 mg/kg of doxycycline for at least 1 day per week for at least 8 weeks.

Embodiment 145. The cell of embodiments 129-145, wherein the cell is administered a dosage of at least about 0.001 mg/kg to about 1000 mg/kg of doxycycline for at least 1 day per week for at least 12 weeks.

Embodiment 147. The cell of embodiments 129-146, wherein the cell is administered a dosage of at least about 0.001 mg/kg to about 1000 mg/kg of doxycycline for at least 1 day per week for at least 26 weeks.

Embodiment 148. The cell of embodiments 129-147, wherein the cell is administered a dosage of at least about 0.001 mg/kg to about 1000 mg/kg of doxycycline for at least 1 day per week for at least 52 weeks.

Embodiment 149. The cell of embodiments 129-148, wherein the cell has increased CpG promoter hypermethylation compared to a cell that does not comprise the polynucleotide.

Embodiment 150. The cell of embodiments 129-149, wherein the cell has increased telomere length compared to a cell that does not comprise the polynucleotide.

Embodiment 151. The cell of embodiments 129-150, wherein the cell has from about 10% to about 50% increased median telomere length compared to a cell that does not comprise the polynucleotide.

Embodiment 152. The cell of embodiments 129-151, wherein the cell has about 30% increased median telomere length compared to a cell that does not comprise the polynucleotide.

Embodiment 153. The cell of embodiments 129-152, wherein the cell has from about 10% to about 50% increased 20^{th} percentile telomere length compared to a cell that does not comprise the polynucleotide.

Embodiment 154. The cell of embodiments 129-153, wherein the cell has about 30% increased 20^{th} percentile telomere length compared to a cell that does not comprise the polynucleotide.

Embodiment 155. The cell of embodiments 129-154, wherein the cell has about 30% increased 20^{th} percentile telomere length compared to baseline.

Embodiment 156. The cell of embodiments 129-155, wherein the cell has about 10% decreased telomeres 3 Kilo base pairs (Kbp) in length compared to a cell that does not comprise the polynucleotide.

Embodiment 157. The cell of embodiments 129-156, wherein the cell has an increased lifespan compared to a cell that was not comprise the polynucleotide.

### INCORPORATION BY REFERENCE

All references, articles, publications, patents, patent publications, and patent applications cited herein are incorporated by reference in their entireties for all purposes. However, mention of any reference, article, publication, patent, patent publication, and patent application cited herein is not, and should not, be taken as an acknowledgement or any form of suggestion that they constitute valid prior art or form part of the common general knowledge in any country in the world.

1. A composition to increase cellular longevity comprising a polyethylenimine (PEI) formulation and a polynucleotide;
wherein the polynucleotide encodes:
one or more heterologous polynucleotide(s) encoding one or more Yamanaka Factors, wherein the one or more polynucleotide(s) encoding the one or more Yamanaka Factors is operably linked to a promoter that is not in nature associated with the one or more polynucleotide(s) encoding the one or more Yamanaka Factors, said promoter directly or indirectly induced;
wherein the polynucleotide expresses the polynucleotide(s) encoding one or more Yamanaka Factors under conditions that induce the promoter operably linked to the polynucleotide(s) encoding the one or more Yamanaka Factors; and
wherein the polynucleotide is bound to the cationic monomers and is substantially located within the core of the PEI formulation.

2. The composition of claim 1, wherein the one or more heterologous polynucleotide(s) encoding one or more Yamanaka Factors:
a) one or more heterologous polynucleotide(s) encoding an octamer-binding transcription factor 4 (OCT4), wherein the one or more polynucleotide(s) encoding the OCT4 is operably linked to an inducible promoter that is not in nature associated with the one or more polynucleotide(s) encoding the OCT4, said promoter directly or indirectly induced;
b) one or more heterologous polynucleotide(s) encoding a Kruppel Like Factor 4 (KLF4), wherein the one or more polynucleotide(s) encoding the KLF4 is operably linked to a promoter that is not in nature associated with the one or more polynucleotide(s) encoding the KLF4, said promoter directly or indirectly induced;
c) one or more heterologous polynucleotide(s) encoding an SRY-box 2 (SOX2), wherein the one or more polynucleotide(s) encoding the SOX2 is operably linked to an inducible promoter that is not in nature associated with the one or more polynucleotide(s) encoding the SOX2, said promoter directly or indirectly induced;
wherein the polynucleotide expresses: the polynucleotide(s) encoding OCT4 under conditions that induce the promoter operably linked to the polynucleotide(s) encoding OCT4; the polynucleotide(s) encoding KLF4 under conditions that induce the promoter operably linked to the polynucleotide(s) encoding KLF4; the polynucleotide(s) encoding SOX2 under conditions that include the promoter operably linked to the polynucleotide(s) encoding SOX2; and
wherein the polynucleotide is bound to the cationic monomers and is substantially located within the core of the PEI formulation;
wherein the promoter operably linked to the polynucleotide(s) encoding OCT4, the promoter operably linked to KLF4, and the promoter operably linked to the polynucleotide(s) encoding SOX2 are the same promoter.

3. The composition of claim 1, wherein at least one polynucleotide is operably linked to a directly or indirectly inducible promoter that is induced by a chemical and/or nutritional inducer, wherein the chemical and/or nutritional inducer is selected from arabinose, IPTG, tetracycline, and rhamnose, and wherein at least one polynucleotide is under the control of a Tet promoter sequence and a Tet repressor polynucleotide.

4. The composition of claim 1, wherein the OCT4 polynucleotide shares at least 80%, at least 85%, at least 90%, at least 95%, at least 98%, at least 99%, or 100% identity to SEQ ID NO: 1, wherein the KLF4 polynucleotide shares at least 80%, at least 85%, at least 90%, at least 95%, at least 98%, at least 99%, or 100% identity to SEQ ID NO: 2, wherein the SOX2 polynucleotide shares at least 80%, at least 85%, at least 90%, at least 95%, at least 98%, at least 99%, or 100% identity to SEQ ID NO: 3, wherein the polynucleotide encodes a OCT4 polypeptide shares at least 80%, at least 85%, at least 90%, at least 95%, at least 98%, at least 99%, or 100% identity to SEQ ID NO: 4, wherein the polynucleotide encodes a KLF4 polypeptide shares at least 80%, at least 85%, at least 90%, at least 95%, at least 98%, at least 99%, or 100% identity to SEQ ID NO: 5, and wherein the polynucleotide encodes a SOX2 polypeptide shares at least 80%, at least 85%, at least 90%, at least 95%, at least 98%, at least 99%, or 100% identity to SEQ ID NO: 6.

5. The composition of claim 1, wherein the composition is administrated to a cell or a subject in need thereof, wherein the PEI formulation and polynucleotide are administered in a single dose and/or multiple doses.

6. The composition of claim 5, wherein the cell is administered a dosage of at least about 0.001 mg/kg to about 1000 mg/kg of doxycycline for at least 1 day per week and water only for the remaining days for about 2 weeks to 52 weeks.

7. The composition of claim 5, wherein the cell has increased CpG promoter hypermethylation compared to baseline, about 10% to about 50% increased median telomere length compared to baseline, about 10% to about 50% increased 20^{th} percentile telomere length compared to baseline, and/or about 10% decreased telomeres 3 Kilo base pairs (Kbp) in length compared to baseline.

8. The composition of claim 5, wherein the composition increases the density of anagen follicles in a subject, increases the density of catagen follicles in a subject, decreases adnexal atrophy in a subject, and/or decreases mononuclear perivascular inflammatory infiltrate in the dermis in a subject.

9. The composition of claim 1, wherein the polynucleotide and PEI comprise a N:P ratio from about 1:1 to about 10:1.

10. A method to increase cellular longevity comprising administering a polyethylenimine (PEI) formulation and a polynucleotide to a subject in need thereof;
wherein the polynucleotide encodes:
one or more heterologous polynucleotide(s) encoding one or more Yamanaka Factors, wherein the one or more polynucleotide(s) encoding the one or more Yamanaka Factors is operably linked to a promoter that is not in nature associated with the one or more polynucleotide(s) encoding the one or more Yamanaka Factors, said promoter directly or indirectly induced;
wherein the polynucleotide expresses the polynucleotide(s) encoding one or more Yamanaka Factors under conditions that induce the promoter operably linked to the polynucleotide(s) encoding the one or more Yamanaka Factors; and
wherein the polynucleotide is bound to the cationic monomers and is substantially located within the core of the PEI formulation.

11. The method of claim 10, wherein the one or more heterologous polynucleotide(s) encoding one or more Yamanaka Factors:
a) one or more heterologous polynucleotide(s) encoding an octamer-binding transcription factor 4 (OCT4), wherein the one or more polynucleotide(s) encoding the OCT4 is operably linked to an inducible promoter that is not in nature associated with the one or more polynucleotide(s) encoding the OCT4, said promoter directly or indirectly induced;
b) one or more heterologous polynucleotide(s) encoding a Kruppel Like Factor 4 (KLF4), wherein the one or more polynucleotide(s) encoding the KLF4 is operably linked to a promoter that is not in nature associated with the one or more polynucleotide(s) encoding the KLF4, said promoter directly or indirectly induced;
c) one or more heterologous polynucleotide(s) encoding an SRY-box 2 (SOX2), wherein the one or more polynucleotide(s) encoding the SOX2 is operably linked to an inducible promoter that is not in nature associated with the one or more polynucleotide(s) encoding the SOX2, said promoter directly or indirectly induced;
wherein the polynucleotide expresses: the polynucleotide(s) encoding OCT4 under conditions that induce the promoter operably linked to the polynucleotide(s) encoding OCT4; the polynucleotide(s) encoding KLF4 under conditions that induce the promoter operably linked to the polynucleotide(s) encoding KLF4; the polynucleotide(s) encoding SOX2 under conditions that include the promoter operably linked to the polynucleotide(s) encoding SOX2; and
wherein the polynucleotide is bound to the cationic monomers and is substantially located within the core of the PEI formulation.

12. The method of claim 10, wherein at least one polynucleotide is operably linked to a directly or indirectly inducible promoter that is induced by a chemical and/or nutritional inducer, wherein the chemical and/or nutritional inducer is selected from arabinose, IPTG, tetracycline, and rhamnose, and wherein at least one polynucleotide is under the control of a Tet promoter sequence and a Tet repressor polynucleotide.

13. The method of claim 10, wherein the OCT4 polynucleotide shares at least 80%, at least 85%, at least 90%, at least 95%, at least 98%, at least 99%, or 100% identity to SEQ ID NO: 1, wherein the KLF4 polynucleotide shares at least 80%, at least 85%, at least 90%, at least 95%, at least 98%, at least 99%, or 100% identity to SEQ ID NO: 2, wherein the SOX2 polynucleotide shares at least 80%, at least 85%, at least 90%, at least 95%, at least 98%, at least 99%, or 100% identity to SEQ ID NO: 3, wherein the polynucleotide encodes a OCT4 polypeptide shares at least 80%, at least 85%, at least 90%, at least 95%, at least 98%, at least 99%, or 100% identity to SEQ ID NO: 4, wherein the polynucleotide encodes a KLF4 polypeptide shares at least 80%, at least 85%, at least 90%, at least 95%, at least 98%, at least 99%, or 100% identity to SEQ ID NO: 5, and wherein the polynucleotide encodes a SOX2 polypeptide shares at least 80%, at least 85%, at least 90%, at least 95%, at least 98%, at least 99%, or 100% identity to SEQ ID NO: 6.

14. The method of claim 10, wherein the PEI formulation and polynucleotide are administered in a single dose and/or multiple doses.

15. The method of claim 10, wherein the subject is administered a dosage of at least about 0.001 mg/kg to about 1000 mg/kg of doxycycline for at least 1 day per week and water only for the remaining days for about 2 weeks to 52 weeks.

16. The method of claim 10, wherein the subject has increased CpG promoter hypermethylation compared to baseline, about 10% to about 50% increased median telomere length compared to baseline, about 10% to about 50% increased 20^{th} percentile telomere length compared to baseline, and/or about 10% decreased telomeres 3 Kilo base pairs (Kbp) in length compared to baseline.

17. The method of claim 10, wherein the polyethylenimine (PEI) formulation and polynucleotide increases the density of anagen follicles in a subject, increases the density of catagen follicles in a subject, decreases adnexal atrophy in a subject, and/or decreases mononuclear perivascular inflammatory infiltrate in the dermis in a subject.

18. The method of claim 10, wherein the polynucleotide and PEI comprise a N:P ratio from about 1:1 to about 10:1.

19. The method of claim 10, wherein the polyethylenimine (PEI) formulation and polynucleotide decreases hair loss in a subject by about 10% and/or increases skin elasticity in a subject by about 10%.

20. A formulation comprising:
a) PEI
b) A polynucleotide encoding:
a. one or more heterologous polynucleotide(s) encoding an Octamer-binding transcription factor 4 (OCT4), wherein the one or more polynucleotide(s) encoding the OCT4 is operably linked to a promoter that is not in nature associated with the one or more polynucleotide(s) encoding the OCT4, said promoter directly or indirectly induced;
b. one or more heterologous polynucleotide(s) encoding a Kruppel Like Factor 4 (KLF4), wherein the one or more polynucleotide(s) encoding the KLF4 is operably linked to a promoter that is not in nature associated with the one or more polynucleotide(s) encoding the KLF4, said promoter directly or indirectly induced;
c. one or more heterologous polynucleotide(s) encoding an SRY-box 2 (SOX2), wherein the one or more polynucleotide(s) encoding the SOX2 is operably linked to an inducible promoter that is not in nature associated with the one or more polynucleotide(s) encoding the SOX2, said promoter directly or indirectly induced; wherein the polynucleotide expresses the polynucleotide(s) encoding OCT4 under conditions that induce the promoter operably linked to the polynucleotide(s) encoding OCT4; the polynucleotide(s) encoding KLF4 under conditions that induce the promoter operably linked to the polynucleotide(s) encoding KLF4; the polynucleotide(s) encoding SOX2 under conditions that include the promoter operably linked to the polynucleotide(s) encoding SOX2
c) a pharmaceutically acceptable carrier

21. A cell comprising a polynucleotide, said polynucleotide encoding:
one or more heterologous polynucleotide(s) encoding one or more Yamanaka Factors, wherein the one or more polynucleotide(s) encoding the one or more Yamanaka Factors is operably linked to a promoter that is not in nature associated with the one or more polynucleotide(s) encoding the one or more Yamanaka Factors, said promoter directly or indirectly induced;
wherein the polynucleotide expresses the polynucleotide(s) encoding one or more Yamanaka Factors under conditions that induce the promoter operably linked to the polynucleotide(s) encoding the one or more Yamanaka Factors.
